(19) United States
(12) Patent Application Publication
(10) Pub. No.: US 2025/0074873 A1
(43) Pub. Date: Mar. 6, 2025

(54) COMPOUNDS

(71) Applicant: Psylo Pty Ltd, Sydney, New South Wales (AU)

(72) Inventors: Samuel Banister, Sydney NSW (AU); William Jorgensen, Sydney NSW (AU); Jinlong Tan, Sydney NSW (AU)

(21) Appl. No.: 18/723,836

(22) PCT Filed: Dec. 23, 2022
(86) PCT No.: PCT/AU2022/051593

§ 371 Date: Jun. 24, 2024

(30) Foreign Application Priority Data
Dec. 24, 2021 (AU) 2021904274

(51) Int. Cl. C07D 209/16; A61K 31/4045; C07D 405/12

(52) U.S. Cl. CPC C07D 209/16; A61K 31/4045; C07D 405/12

(57) ABSTRACT

The present disclosure relates generally to compounds, their methods of synthesis, and their use in the treatment of mental illness or central nervous system disorders.

This application claims priority to Australian provisional application no 2021904274 (filed 24 Dec. 2021), which is entirely incorporated herein by reference.

### FIELD OF THE INVENTION

The present disclosure relates generally to novel compounds, their methods of synthesis, and their use in the treatment of mental illness or central nervous system disorders.

### BACKGROUND OF THE INVENTION

Mental illness covers many neuropsychiatric disorders which cause enormous burden on the lives of their sufferers. Diagnoses such as treatment resistant depression, major depressive disorder, eating disorders, substance abuse disorders, post-traumatic stress disorder, obsessive compulsive disorder, attention deficit disorders, schizophrenia, and others can cause such devastating symptoms that many sufferers lose the capability of leading a normal life.

A variety of serotonergic drugs such as antidepressants, serotonin reuptake inhibitors, monoamine oxidase inhibitors, selective serotonin reuptake inhibitors, and others are commercially available to treat mental illnesses. Unfortunately, in many indications, these therapeutics provide limited benefit when compared to a placebo. Additionally, these therapeutics can result in a wide range of side effects including loss of libido, insomnia, fatigue, weight gain, and others. In spite of their limited efficacy, these drugs continue to be used to treat neuropsychiatric conditions as well as a broad range of auxiliary medical indications. There have been limited advances in new treatment options since many of these drugs were released, and the pharmaceutical industry has come under increased financial pressure to de-emphasise neuroscience programmes entirely. The unmet need for more efficacious mental health treatment is on the rise, and the global COVID-19 pandemic is likely to increase disease burden around the world.

In the 1950s and 1960s, the use of psychedelic drugs to treat various mental illnesses was extensively explored, and these substances showed promise as treatments for many diseases of the central nervous system (CNS). Following decades of prohibition, scientific research into the application of psychedelics as treatments for mental illnesses has been gaining momentum. The serotonergic psychedelic agent psilocybin has been designated a Breakthrough Therapy by the FDA for the treatment of major depressive disorder (2019) and treatment-resistant depression (2018). Psilocybin is the prodrug compound produced by more than many species of mushrooms known collectively as psilocybin mushrooms or “magic mushrooms”. Psilocybin is rapidly metabolized to the bioactive compound psilocin, which produces a state of altered consciousness including changes in perception, visual hallucinations, and distorted sense of space, time, and self. Many patients report spiritual or “mystical” experiences which have profound and lasting impact on the patients' mood and behaviour. Psilocybin has shown promise in more than 50 clinical trials for neuropsychiatric indications, including numerous anxiety disorders, obsessive-compulsive disorder, anorexia nervosa, alcohol dependence, and tobacco addiction. Psilocybin and other psychedelic compounds such as N,N-dimethyltryptamine (DMT) and 5-methoxy-N,N-dimethyltryptamine (5-MeO-DMT) have both immediate and persistent effects on mental state, with the latter extending far beyond the duration of action, possibly as a result of their ability to incite increased neuroplasticity, promote neural outgrowth, and increase spine density of the synaptic neurons in the brain.

To date, psilocybin remains classified as a controlled substance and/or drug of abuse in most countries under national drug laws. However, clinical investigations have recently led to increased awareness of the potential for psychedelic drugs as breakthrough therapies to treat CNS diseases of enormous unmet medical need.

Despite its therapeutic potential, psilocybin and other psychedelics remain scheduled drugs of abuse in most countries and the commercial path to market for these drugs as medicines is uncertain. As an adjunct to psychotherapy, the long duration of action of psilocybin and LSD make treatment sessions costly and impractical for broad implementation. In spite of a long history of safe human use, several adverse events have been reported in clinical trials, and it is possible that these may be attributed to signalling bias at 5-HT2A (the primary target) or off-target activity at, for example, 5-HT2B receptors (a cardiac liability antitarget) or 5-HT1A (an anxiolytic target) or 5-HT2C receptors (a disease-relevant target for obesity and some genetic epilepsies, for example). Naturally-occurring psychedelics provide important lead structures for a new generation of neurotherapeutic agents with novel mechanisms of action and/or superior clinical efficacy to currently available neuropsychiatric medications.

In view of the foregoing there is an ongoing need to develop new compounds which may be useful in the treatment of mental illness or central nervous system disorders.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect the present disclosure provides a compound of formula (I):

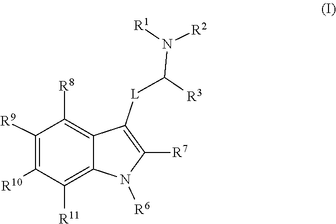

or a pharmaceutically acceptable salt, solvate, tautomer, N-oxide, stereoisomer, metabolite, polymorph and/or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl, said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O)R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹², said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein one of (B) and (C) apply:
(B)
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₁ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl;
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃,
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form

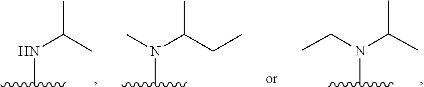

and R³ and R⁶ are each hydrogen, then R⁹ is not OCH₃;
when R¹ and R² together with the nitrogen to which they are attached form

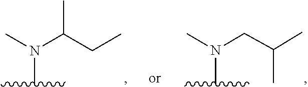

and R³ and R⁶ are each hydrogen, then R⁸ is not OH;
when R¹ and R² together with the nitrogen to which they are attached form

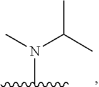 [SMILES: *N(C)C(C)C]

and R³ and R⁶ are each hydrogen, then R⁸ is not OH, R⁹ is not CH₃ or OCH₃, and R¹⁰ is not OCH₃;
when R¹ and R² together with the nitrogen to which they are attached form

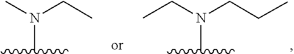

and R³ and R⁶ are each hydrogen, then R⁷, R⁸, R⁹, R¹⁰ and R¹¹ are not selected from OH, OCH₃, OC(O)CH₃, OP(O)(OH)₂, NH₂, halogen, CH₃, CN and CF₃; when R¹ and R² together with the nitrogen to which they are attached form any one of

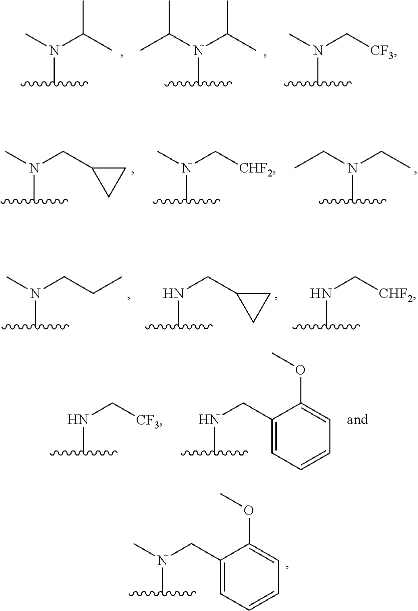

R³ is hydrogen, and R⁶ is methyl, then R⁸ is not OH or OBn;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH or OBn;
when R¹ and R² are each methyl, R³ is hydrogen, and R⁶ is hydrogen or CH₂P(O)(OH)₂, then R^{a} is not selected from

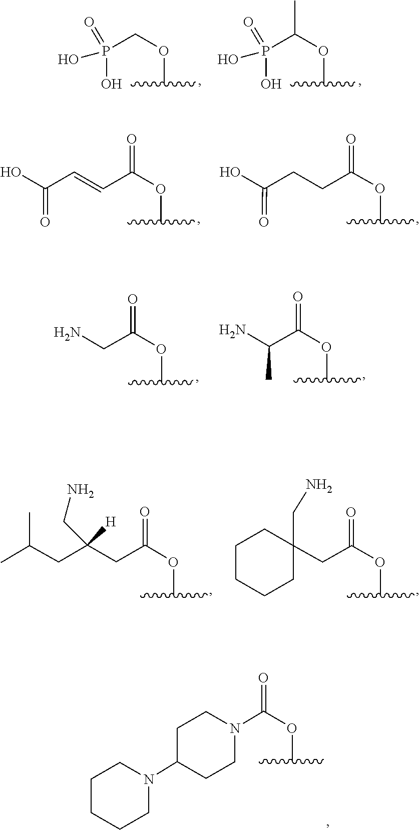

and OC(O)N(CH₃)₂; and
when R¹, R² and R³ together with the atoms to which they are attached form

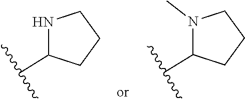

and R⁶ is hydrogen or CH₂P(O)(OH)₂, then R⁸ is not selected from OH

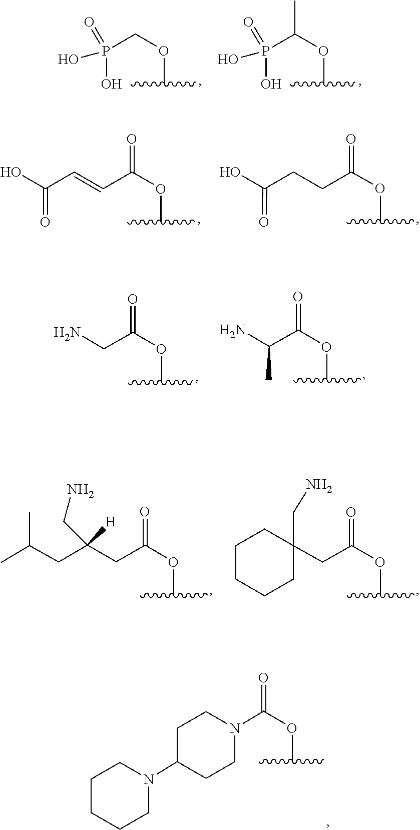

and OC(O)N(CH₃)₂;
(C) R⁷, R⁸, R⁹, R¹⁰ and R¹¹ are each hydrogen,
wherein:
when R³ and R⁶ are each hydrogen, then R¹ and R² are not each methyl, ethyl, propyl, isopropyl, cyclopropyl or

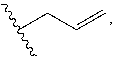 [SMILES: *CC=C]

and R¹ and R² together with the nitrogen to which they are attached do not form pyrrolidyl, piperidyl or 2,5-dimethylpyrrolyl;
when R⁶ is hydrogen, and R³ is methyl, then R¹ and R² are not each hydrogen;
when R³ and R⁶ are each hydrogen, and one of R¹ and R² is methyl, then the other of R¹ and R² is not propyl, isopropyl, cyclopropyl, methylenecyclopropyl,

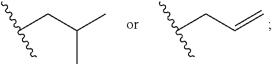

when R³ and R⁶ are each hydrogen, and one of R¹ and R² is ethyl or propyl, then the other of R¹ and R² is not isopropyl, cyclopropyl, methylenecyclopropyl,

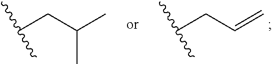

and
when R³ and R⁶ are each hydrogen, and one of R¹ and R² is isopropyl, then the other of R¹ and R² is not propyl, cyclopropyl, methylenecyclopropyl,

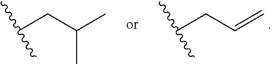

The disclaimers of the first aspect of the invention, and any selection or combination of those disclaimers, may also apply to other embodiments.

In another aspect of the present disclosure there is provided a medicament comprising a compound of formula (I) according to any one of the herein disclosed embodiments, or a pharmaceutically acceptable salt, solvate, tautomer, N-oxide, stereoisomer, metabolite, polymorph or prodrug thereof.

In another aspect of the present disclosure there is provided a pharmaceutical composition comprising a compound of formula (I) according to any one of the herein disclosed embodiments, or a pharmaceutically acceptable salt, solvate, tautomer, N-oxide, stereoisomer, metabolite, polymorph or prodrug thereof, and a pharmaceutically acceptable excipient.

In another aspect the present disclosure provides a pharmaceutical composition comprising a compound according to any one of the herein disclosed embodiments, or a pharmaceutically acceptable salt, solvate, tautomer, N-oxide, stereoisomer, metabolite, polymorph or prodrug thereof, an additional therapeutic agent, and a pharmaceutically acceptable excipient.

In another aspect of the present disclosure there is provided a method of treating a disease, disorder or condition by activation of a serotonin receptor, the method comprising administering to a subject in need thereof a compound of formula (I):

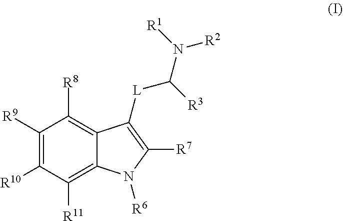

or a pharmaceutically acceptable salt, solvate, tautomer, N-oxide, stereoisomer, metabolite, polymorph or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O)R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²; said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein one of (B) and (C) apply:
(B)
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴; alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form

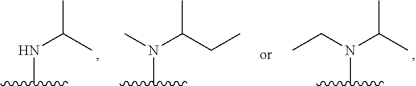

and R³ and R⁶ are each hydrogen, then R⁹ is not OCH₃; when R¹ and R² together with the nitrogen to which they are attached form

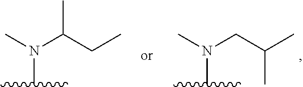

and R³ and R⁶ are each hydrogen, then R⁸ is not OH;
when R¹ and R² together with the nitrogen to which they are attached form

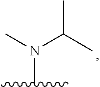 [SMILES: *N(C)C(C)C]

and R³ and R⁶ are each hydrogen, then R⁸ is not OH, R⁹ is not CH₃ or OCH₃, and R¹⁰ is not OCH₃;
when R¹ and R² together with the nitrogen to which they are attached form any one of

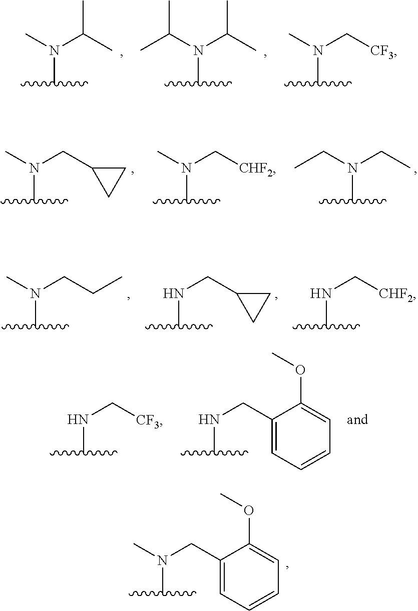

R³ is hydrogen, and
R⁶ is methyl, then R⁸ is not OH;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH;
when R¹ and R² are each methyl, R³ is hydrogen, and R⁶ is hydrogen or CH₂P(O)(OH)₂,

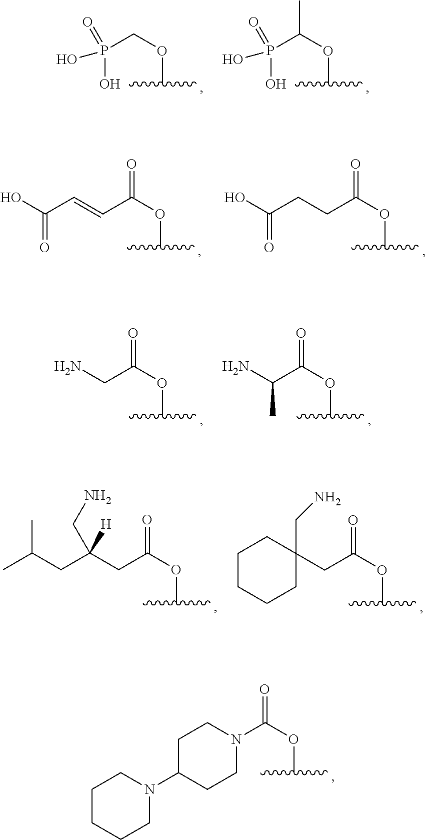

and OC(O)N(CH₃)₂; and
when R¹, R² and R³ together with the atoms to which they are attached form

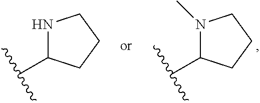

and R⁶ is hydrogen or CH₂P(O)(OH)₂, then R⁸ is not selected from

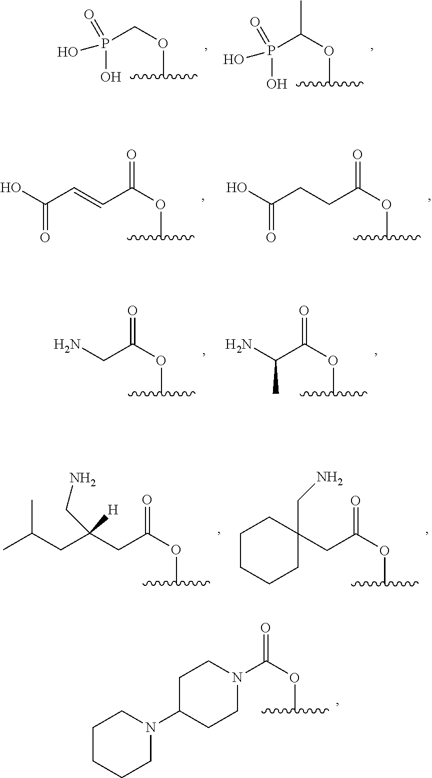

and OC(O)N(CH₃)₂;
(C) R⁷, R⁸, R⁹, R¹⁰ and R¹¹ are each hydrogen,
wherein:
when R⁶ and R³ are each hydrogen, then R¹ and R² are not each methyl, and R¹ and R² together with the nitrogen to which they are attached do not form

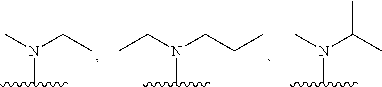

pyrrolidyl, piperidyl or 2,5-dimethylpyrrolyl; and
when R⁶ is hydrogen, and R³ is methyl, then R¹ and R² are not each hydrogen.

Further aspects of the present invention and further embodiments of the aspects described in the preceding paragraphs will become apparent from the following description, given by way of example and with reference to the accompanying drawings.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

In a first aspect this disclosure provides a compound of formula (I) as described above.

In some embodiments,
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl;
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein:
when R¹ and R² are each methyl, and R³ and R⁶ are each hydrogen, then R⁸ is not OH or OCH₃, and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form

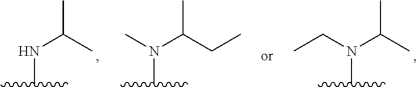

and R³ and R⁶ are each hydrogen, then R⁹ is not OCH₃;
when R¹ and R² together with the nitrogen to which they are attached form

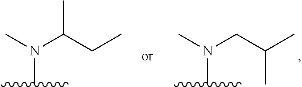

and R³ and R⁶ are each hydrogen, then R⁸ is not OH;
when R¹ and R² together with the nitrogen to which they are attached form

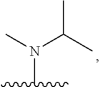 [SMILES: *N(C)C(C)C]

and R³ and R⁶ are each hydrogen, then R⁸ is not OH, R⁹ is not CH₃ or OCH₃, and R¹⁰ is not OCH₃;
when R¹ and R² together with the nitrogen to which they are attached form

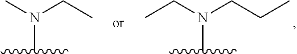

and R³ and R⁶ are each hydrogen, then R⁷, R⁸, R⁹, R¹⁰ and R¹¹ are not selected from OH, OCH₃, OC(O)CH₃, OP(O)(OH)₂, NH₂, halogen, CH₃, CN and CF₃;
when R¹ and R² together with the nitrogen to which they are attached form any one of

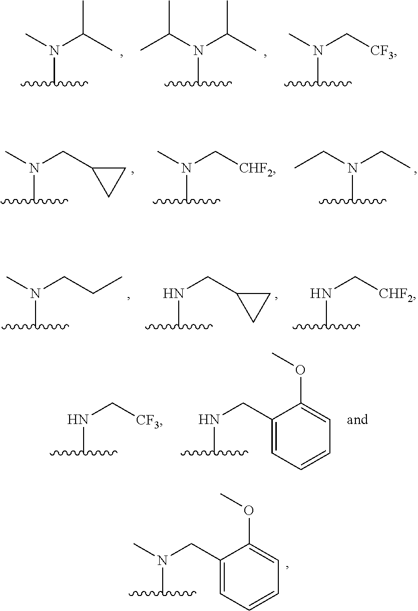

R³ is hydrogen, and R⁶ is methyl, then R⁸ is not OH or OBn;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH or OBn;
when R¹ and R² are each methyl, R³ is hydrogen, and R⁶ is hydrogen or CH₂P(O)(OH)₂, then R⁸ is not selected from

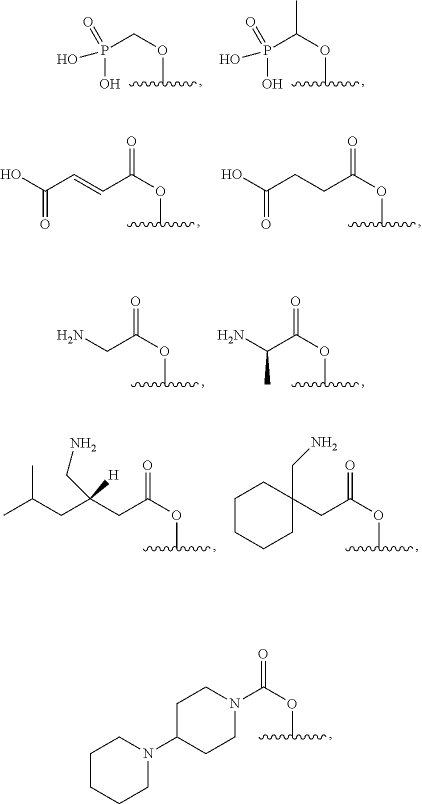

and OC(O)N(CH₃)₂; and
when R¹, R² and R³ together with the atoms to which they are attached form

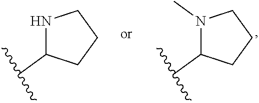

and R⁶ is hydrogen or CH₂P(O)(OH)₂, then R⁸ is not selected from

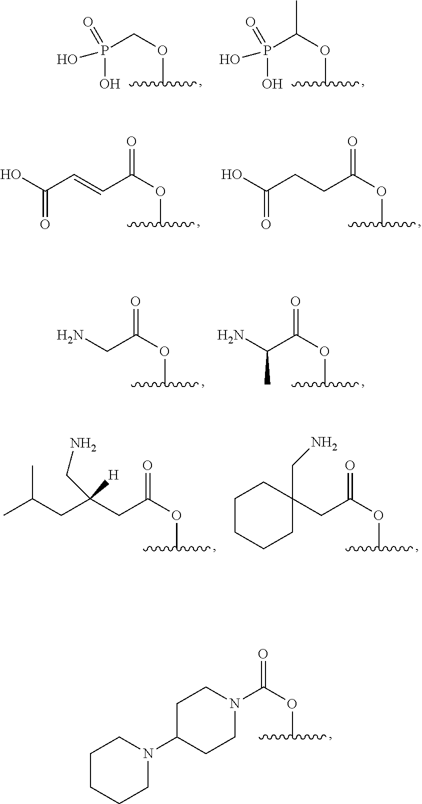

and OC(O)N(CH₃)₂.

In some embodiments,
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₁ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form

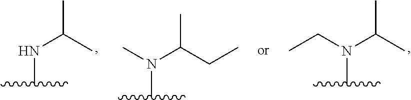

and R³ and R⁶ are each hydrogen, then R⁹ is not OCH₃;
when R¹ and R² together with the nitrogen to which they are attached form

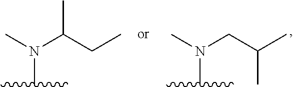

and R³ and R⁶ are each hydrogen, then R⁸ is not OH;
when R¹ and R² together with the nitrogen to which they are attached form

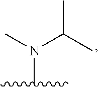 [SMILES: *N(C)C(C)C]

and R³ and R⁶ are each hydrogen, then R⁸ is not OH, R⁹ is not CH₃ or OCH₃, and R¹⁰ is not OCH₃;
when R¹ and R² together with the nitrogen to which they are attached form

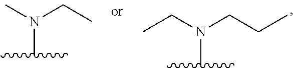

and R³ and R⁶ are each hydrogen, then R⁷, R⁸, R⁹, R¹⁰ and R¹¹ are not selected from OH, OCH₃, OC(O)CH₃, OP(O)(OH)₂, NH₂, halogen, CH₃, CN and CF₃;
when R¹ and R² together with the nitrogen to which they are attached form any one of

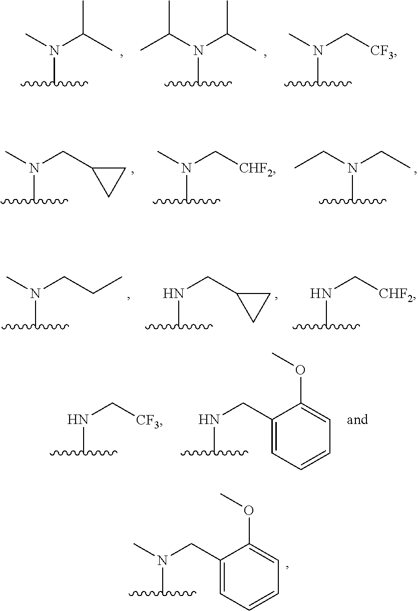

R³ is hydrogen, and R⁶ is methyl, then R⁸ is not OH or OBn;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH or OBn;
when R¹ and R² are each methyl, R³ is hydrogen, and R⁶ is hydrogen or CH₂P(O)(OH)₂, then R⁸ is not selected from

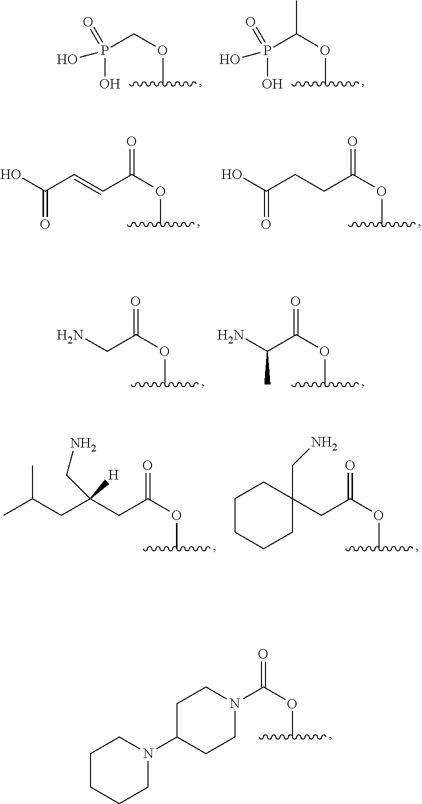

and OC(O)N(CH₃)₂; and
when R¹, R² and R³ together with the atoms to which they are attached form

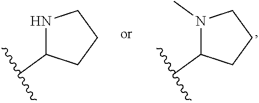

and R⁶ is hydrogen or CH₂P(O)(OH)₂, then R⁸ is not selected from OH

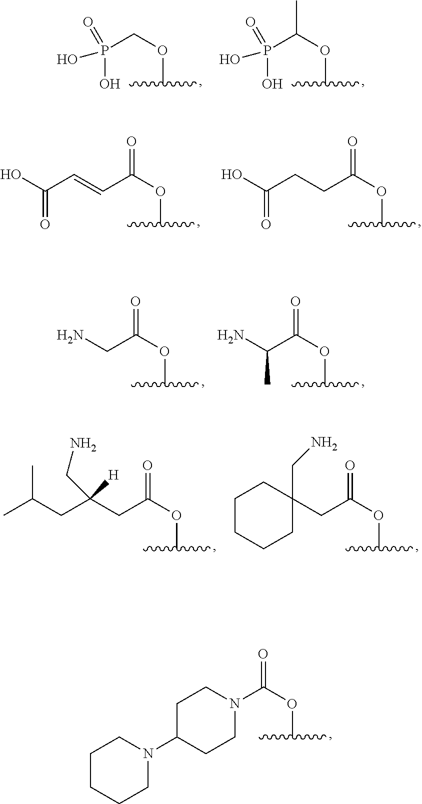

and OC(O)N(CH₃)₂.

In some embodiments:
(i) one of R⁷, R⁸, R⁹, R¹⁰ and R¹¹ is selected from C₁₋₆ haloalkyl and OC₁₋₆ haloalkyl, wherein the C₁₋₆ haloalkyl is not CF₃ when R¹ and R² together with the nitrogen to which they are attached form

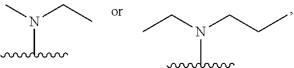

and R³ and R⁶ are each hydrogen; and
(ii) the other of R⁷, R⁸, R⁹, R¹⁰ and R¹¹ are each hydrogen.

In some embodiments, wherein R⁷, R⁸, R⁹, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl, and
wherein:
when R¹ and R² are each methyl, R³ is hydrogen, R⁶ is selected from hydrogen, methyl, ethyl and propyl, and one of R⁹, R¹⁰ and R¹¹ are fluoro and the other of R⁹, R¹⁰ and R¹¹ are hydrogen, then R⁸ is not selected from OH, OCH₃, OCH₂CH₃ and OCH₂CH₂CH₃; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃ and OCH₂CH₂CH₃.

In some embodiments, wherein R⁷, R⁸, R⁹, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl, and
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ and R¹¹ are fluoro and the other of R⁹, R¹⁰ and R¹¹ are hydrogen, then R⁸ is not selected from OH, OCH₃, OCH₂CH₃ and OCH₂CH₂CH₃; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃ and OCH₂CH₂CH₃.

In some embodiments, R⁷, R⁸, R⁹, R¹⁰ and R¹¹ are each hydrogen.

In some embodiments, R⁷, R⁸, R⁹, R¹⁰ and R¹¹ are each hydrogen,
wherein:
when R³ and R⁶ are each hydrogen, then R¹ and R² are not each methyl, ethyl, propyl, isopropyl, cyclopropyl

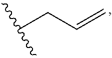 [SMILES: *CC=C]

and R¹ and R² together with the nitrogen to which they are attached do not form pyrrolidyl, piperidyl or 2,5-dimethylpyrrolyl;
when R⁶ is hydrogen, and R³ is methyl, then R¹ and R² are not each hydrogen;
when R³ and R⁶ are each hydrogen, and one of R¹ and R² is methyl, then the other of R¹ and R² is not propyl, isopropyl, cyclopropyl, methylenecyclopropyl,

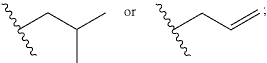

when R³ and R⁶ are each hydrogen, and one of R¹ and R² is ethyl or propyl, then the other of R¹ and R² is not isopropyl, cyclopropyl, methylenecyclopropyl, and

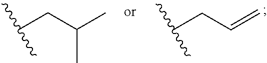

when R³ and R⁶ are each hydrogen, and one of R¹ and R² is isopropyl, then the other of R¹ and R² is not propyl, cyclopropyl, methylenecyclopropyl,

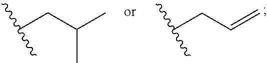

In some embodiments, R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl and C₄₋₁₄ alkylenecycloalkyl.

In some embodiments, R¹ and R² are each independently selected from C₁₋₄ alkyl, C₃₋₈ cycloalkyl and C₃-C₈ heterocycloalkyl. In some embodiments, R¹ and R² are each independently selected from C₁₋₄ alkyl.

In some embodiments, R¹ and R² are the same.

In some embodiments, R¹ and R² are different.

In some embodiments, one or both of R¹ and R² is branched C₁-C₆ alkyl.

In some embodiments, at least one of R¹ and R² is methyl.

In some embodiments, at least one of R¹ and R² is ethyl.

In some embodiments, at least one of R¹ and R² is propyl, preferably iso-propyl.

In some embodiments, at least one of R¹ and R² is butyl.

In some embodiments, R¹ and R², together with the nitrogen to which they are attached, form any one of the following:

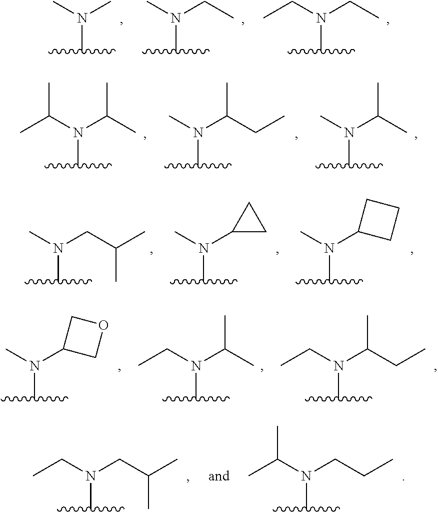

In some embodiments, R¹ and R², together with the nitrogen to which they are attached, form any one of the following:

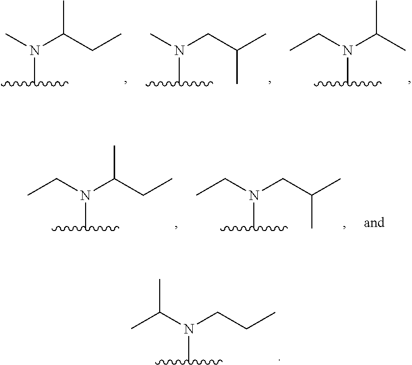

In some embodiments, R¹ and R² are combined with the atoms to which they are attached to form C₃₋₆ heterocycloalkyl, said C₃₋₆ heterocycloalkyl being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴, (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

In some embodiments, R³ is hydrogen.

In some embodiments, R³ and one of R¹ and R² are combined with the atoms to which they are attached to form a C₃₋₈ heterocycloalkyl, said C₅₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

In some embodiments, L is C₁₋₄ alkylene.

In some embodiments, L is methylene.

In some embodiments, R⁶ is selected from hydrogen and C₁₋₆ alkyl.

In some embodiments, R⁶ is hydrogen.

In some embodiments, the compound of formula (I) is selected from any one of the compound of Table 1, for example compounds P4-P5, P15-P61 and P119-P164 or a pharmaceutically acceptable salt, solvate, tautomer, N-oxide, stereoisomer, metabolite, polymorph or prodrug thereof. In some embodiments, the compound of formula (I) is selected from any one of compounds P4-P5, P15-P19, P23-P61 and P119-P164 or a pharmaceutically acceptable salt, solvate, tautomer, N-oxide, stereoisomer, metabolite, polymorph or prodrug thereof. In some embodiments, the compound of formula (I) is selected from any one of compounds P20-P22 or a pharmaceutically acceptable salt, solvate, tautomer, N-oxide, stereoisomer, metabolite, polymorph or prodrug thereof. In some embodiments, the compound of formula (I) is selected from any one of compounds P136, P137, P162 and P163 or a pharmaceutically acceptable salt, solvate, tautomer, N-oxide, stereoisomer, metabolite, polymorph or prodrug thereof.

In some embodiments, the compound of formula (I) is not the following compound:

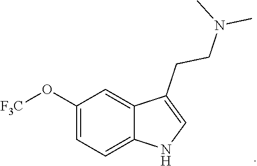 [SMILES: CN(C)CCc1c[nH]c2ccc(OC(F)(F)F)cc12]

In another aspect of the present disclosure there is provided a medicament comprising a compound of formula (I) according to any one of the herein disclosed embodiments, or a pharmaceutically acceptable salt, solvate, tautomer, N-oxide, stereoisomer, metabolite, polymorph or prodrug thereof.

In another aspect of the present disclosure there is provided a pharmaceutical composition comprising a compound of formula (I) according to any one of the herein disclosed embodiments, or a pharmaceutically acceptable salt, solvate, tautomer, N-oxide, stereoisomer, metabolite, polymorph or prodrug thereof, and a pharmaceutically acceptable excipient.

In another aspect the present disclosure provides a pharmaceutical composition comprising a compound according to any one of the herein disclosed embodiments, or a pharmaceutically acceptable salt, solvate, tautomer, N-oxide, stereoisomer, metabolite, polymorph or prodrug thereof, an additional therapeutic agent, and a pharmaceutically acceptable excipient.

In another aspect of the present disclosure there is provided a method of treating a disease, disorder or condition by activation of a serotonin receptor, the method comprising administering to a subject in need thereof a compound of formula (I):

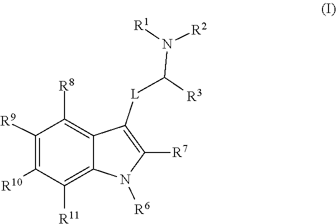

or a pharmaceutically acceptable salt, solvate, tautomer, N-oxide, stereoisomer, metabolite, polymorph or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O)R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²,
said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²,
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein one of (B) and (C) apply:
(B)
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form

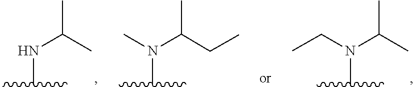

and R³ and R⁶ are each hydrogen, then R⁹ is not OCH₃;
when R¹ and R² together with the nitrogen to which they are attached form

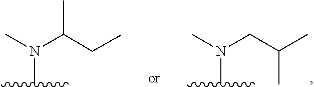

and R³ and R⁶ are each hydrogen, then R⁸ is not OH;
when R¹ and R² together with the nitrogen to which they are attached form

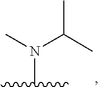 [SMILES: *N(C)C(C)C]

and R³ and R⁶ are each hydrogen, then R⁸ is not OH, R⁹ is not CH₃ or OCH₃, and R¹⁰ is not OCH₃;
when R¹ and R² together with the nitrogen to which they are attached form any one of

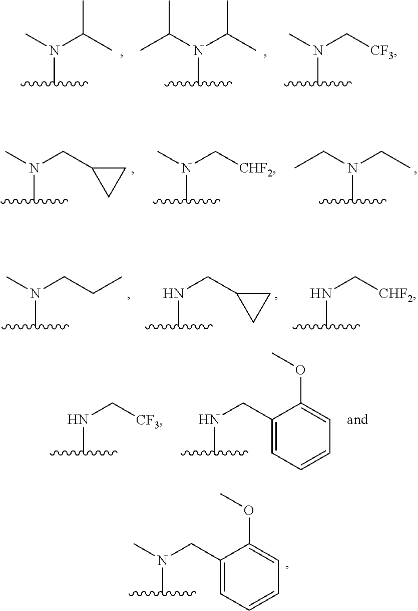

R³ is hydrogen, and R⁶ is methyl, then R⁸ is not OH;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH;
when R¹ and R² are each methyl, R³ is hydrogen, and R⁶ is hydrogen or CH₂P(O)(OH)₂, then R⁸ is not selected from

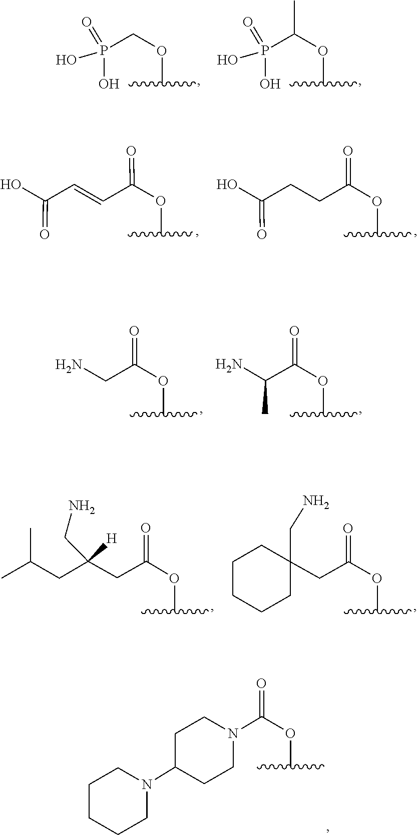

and OC(O)N(CH₃)₂; and
when R¹, R² and R³ together with the atoms to which they are attached form

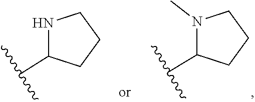

and R⁶ is hydrogen or CH₂P(O)(OH)₂, then R⁸ is not selected from

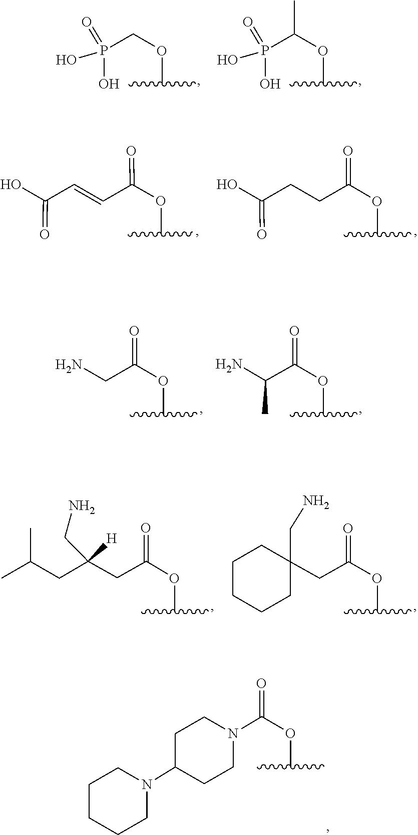

and OC(O)N(CH₃)₂;
(C) R⁷, R⁸, R⁹, R¹⁰ and R¹¹ are each hydrogen,
wherein:
when R⁶ and R³ are each hydrogen, then R¹ and R² are not each methyl, and R¹ and R² together with the nitrogen to which they are attached do not form

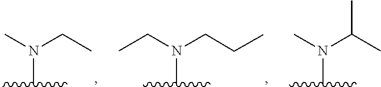

pyrrolidyl, piperidyl or 2,5-dimethylpyrrolyl; and
when R⁶ is hydrogen, and R³ is methyl, then R¹ and R² are not each hydrogen.

In some embodiments of the method, R⁷, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O)R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in any one of the foregoing paragraphs.

In some embodiments of the method, R⁷, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl.

In some embodiments of the method, R⁷, R¹⁰ and R¹¹ are each hydrogen.

In some embodiments of the method, R⁸ is selected from halogen, C₁₋₆ alkyl and OR¹³ wherein R¹³ is selected from hydrogen, C₁₋₆ alkyl and C₁₋₆ haloalkyl.

In some embodiments of the method, R⁹ is selected from halogen, C₁₋₆ alkyl and OR¹³ wherein R¹³ is selected from hydrogen, C₁₋₆ alkyl and C₁₋₆ haloalkyl.

In some embodiments of the method,
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl;
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form

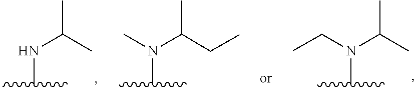

and R³ and R⁶ are each hydrogen, then R⁹ is not OCH₃;
when R¹ and R² together with the nitrogen to which they are attached form

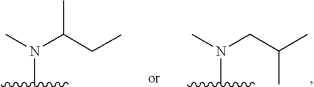

and R³ and R⁶ are each hydrogen, then R⁸ is not OH;
when R¹ and R² together with the nitrogen to which they are attached form

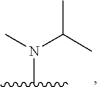 [SMILES: *N(C)C(C)C]

and R³ and R⁶ are each hydrogen, then R⁸ is not OH, R⁹ is not CH₃ or OCH₃, and R¹⁰ is not OCH₃;
when R¹ and R² together with the nitrogen to which they are attached form any one of

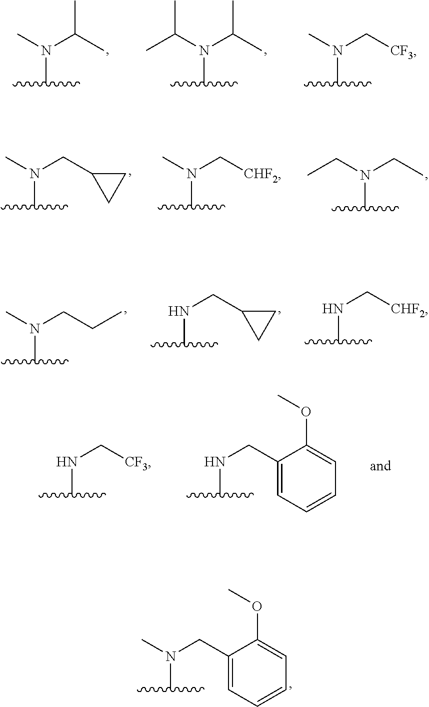

R³ is hydrogen, and R⁶ is methyl, then R⁸ is not OH;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH;
when R¹ and R² are each methyl, R³ is hydrogen, and R⁶ is hydrogen or CH₂P(O)(OH)₂, then R^{a} is not selected from

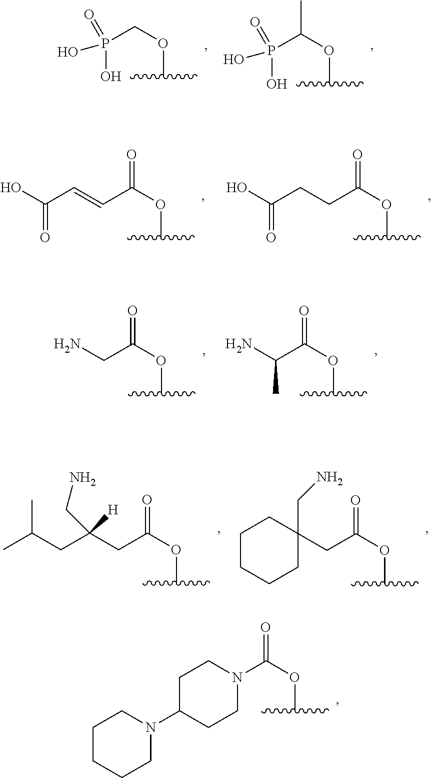

and OC(O)N(CH₃)₂; and
when R¹, R² and R³ together with the atoms to which they are attached form

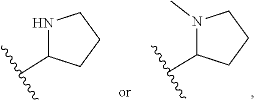

and R⁵ is hydrogen or CH₂P(O)(OH)₂, then R⁸ is not selected from OH

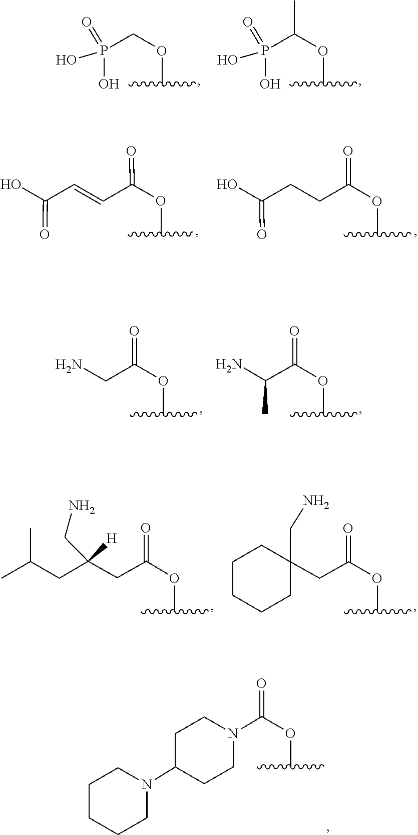

and OC(O)N(CH₃)₂.

In some embodiments of the method,
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
   when R¹ and R² together with the nitrogen to which they are attached form

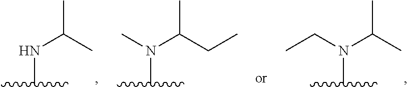

and R³ and R⁶ are each hydrogen, then R⁹ is not OCH₃;
when R¹ and R² together with the nitrogen to which they are attached form

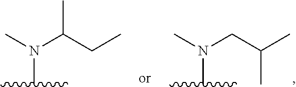

and R³ and R⁶ are each hydrogen, then R⁸ is not OH;
when R¹ and R² together with the nitrogen to which they are attached form

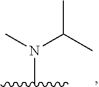 [SMILES: *N(C)C(C)C]

and R³ and R⁶ are each hydrogen, then R⁸ is not OH, R⁹ is not CH₃ or OCH₃, and R¹⁰ is not OCH₃;
when R¹ and R² together with the nitrogen to which they are attached form any one of

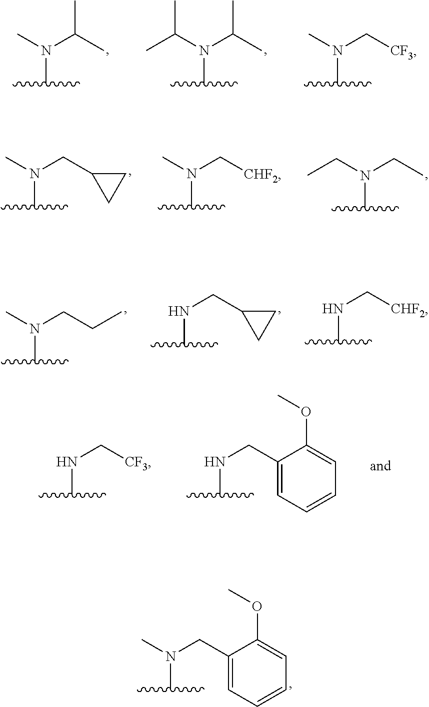

R³ is hydrogen, and R⁶ is methyl, then R⁸ is not OH;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH;
when R¹ and R² are each methyl, R³ is hydrogen, and R⁶ is hydrogen or CH₂P(O)(OH)₂, then R⁸ is not selected from

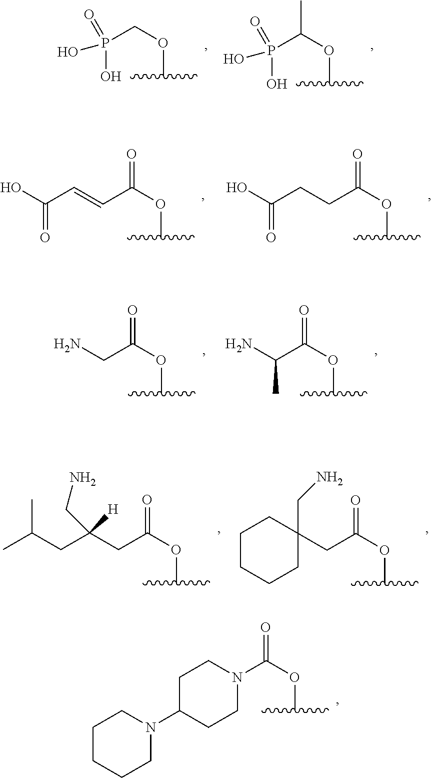

and OC(O)N(CH₃)₂; and
when R¹, R² and R³ together with the atoms to which they are attached form

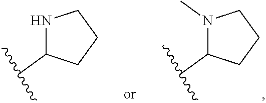

and R⁶ is hydrogen or CH₂P(O)(OH)₂, then R⁸ is not selected from

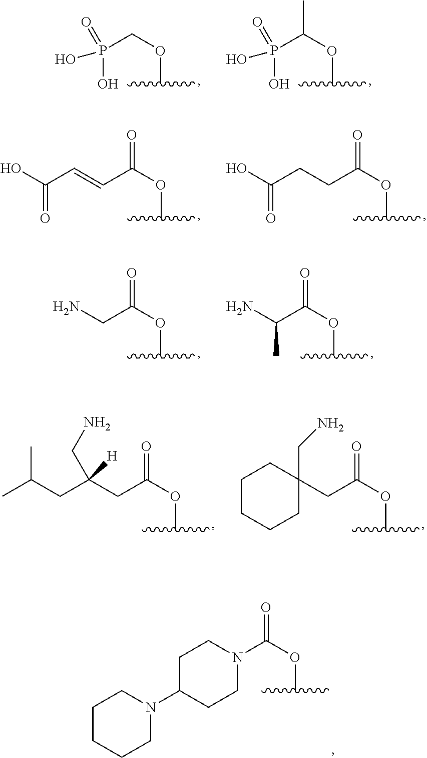

and OC(O)N(CH₃)₂.

In some embodiments of the method:
(i) one of R⁷, R⁸, R⁹, R¹⁰ and R¹¹ is selected from C₁₋₆ haloalkyl and OC₁₋₆ haloalkyl, and
(ii) the other of R⁷, R⁸, R⁹, R¹⁰ and R¹¹ are each hydrogen.

In some embodiments of the method R⁷, R⁸, R⁹, R¹⁰ and R¹¹ are each hydrogen,
wherein:
when R⁶ and R³ are each hydrogen, then R¹ and R² are not each methyl, and R¹ and R² together with the nitrogen to which they are attached do not form

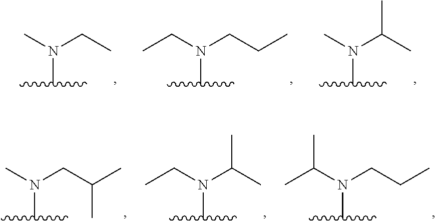

pyrrolidyl, piperidyl or 2,5-dimethylpyrrolyl; and
when R⁶ is hydrogen, and R³ is methyl, then R¹ and R² are not each hydrogen.

In some embodiments of the method, R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl and C₄₋₁₄ alkylenecycloalkyl.

In some embodiments of the method, R¹ and R² are each independently selected from C₁₋₄ alkyl.

In some embodiments of the method, R¹ and R², together with the nitrogen to which they are attached, form any one of the following:

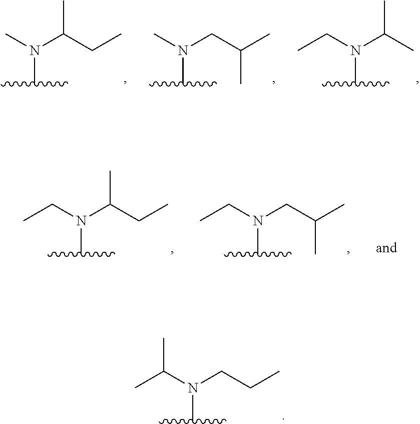

In some embodiments of the method, R¹ and R² are combined with the atoms to which they are attached to form C₃₋₆ heterocycloalkyl, said C₃₋₆ heterocycloalkyl being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴, (0), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

In some embodiments of the method, R³ is hydrogen.

In some embodiments of the method, R³ and one of R¹ and R² are combined with the atoms to which they are attached to form a C₃₋₈ heterocycloalkyl, said C₅₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴, wherein R⁴ is as defined in any one of the foregoing paragraphs.

In some embodiments of the method, L is C₁₋₄ alkylene.

In some embodiments of the method, L is methylene.

In some embodiments of the method, R⁶ is selected from hydrogen and C₁ alkyl.

In some embodiments of the method, R⁶ is hydrogen.

In some embodiments of the method, the compound of formula (I) is selected from any one of the compounds of Table 1 or a pharmaceutically acceptable salt, solvate, tautomer, N-oxide, stereoisomer, metabolite, polymorph or prodrug thereof.

In some embodiments of the method, the compound of formula (I) is selected from any one of the following:

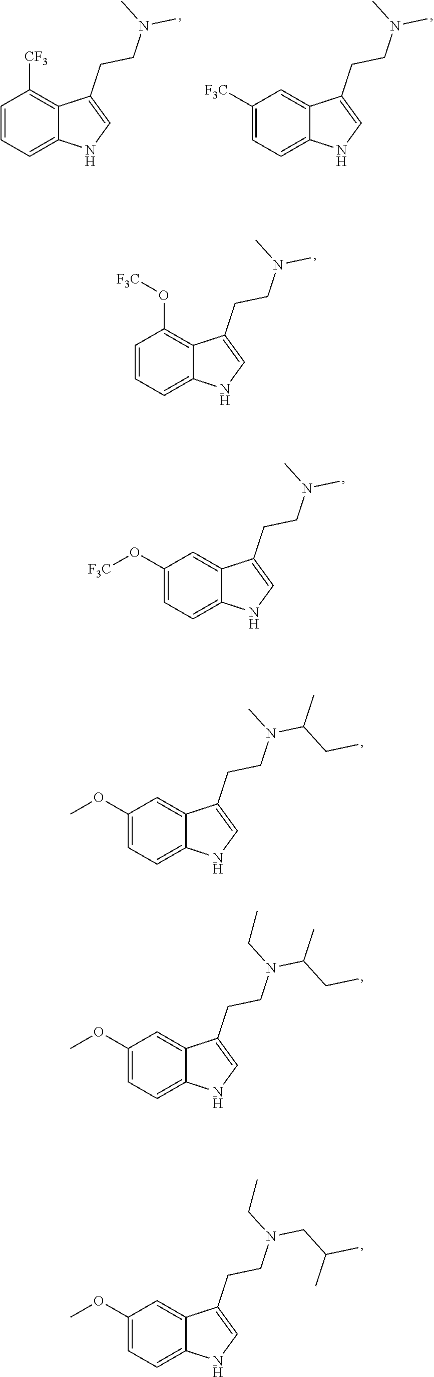

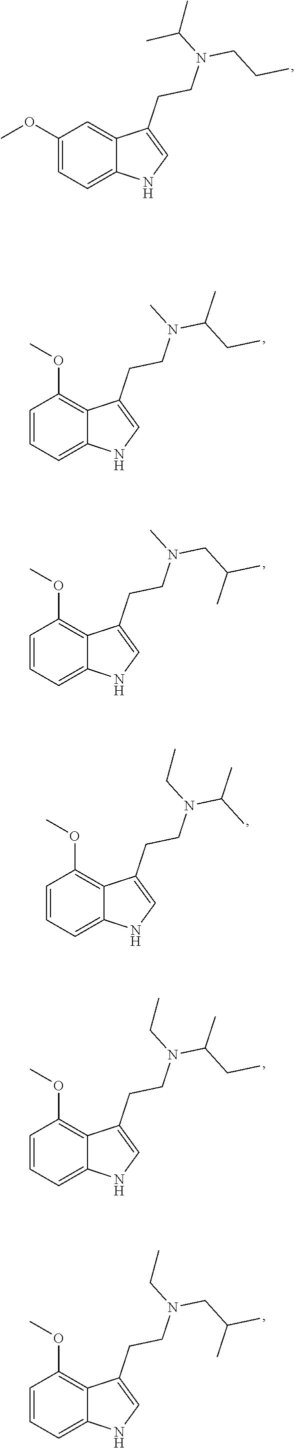 [SMILES: CCCN(CCc1c[nH]c2ccc(OC)cc12)C(C)C ; CCC(C)N(C)CCc1c[nH]c2cccc(OC)c12 ; COc1cccc2[nH]cc(CCN(C)CC(C)C)c12 ; CCN(CCc1c[nH]c2cccc(OC)c12)C(C)C ; CCC(C)N(CC)CCc1c[nH]c2cccc(OC)c12 ; CCN(CCc1c[nH]c2cccc(OC)c12)CC(C)C]

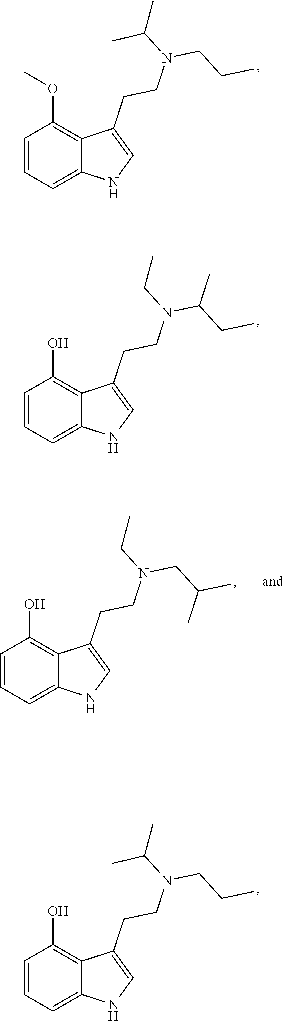 [SMILES: CCCN(CCc1c[nH]c2cccc(OC)c12)C(C)C ; CCC(C)N(CC)CCc1c[nH]c2cccc(O)c12 ; CCN(CCc1c[nH]c2cccc(O)c12)CC(C)C ; CCCN(CCc1c[nH]c2cccc(O)c12)C(C)C]

or a pharmaceutically acceptable salt, solvate, tautomer, N-oxide, stereoisomer, metabolite, polymorph or prodrug thereof.

In some embodiments of the method, the compound of formula (I) is selected from any one of the following:

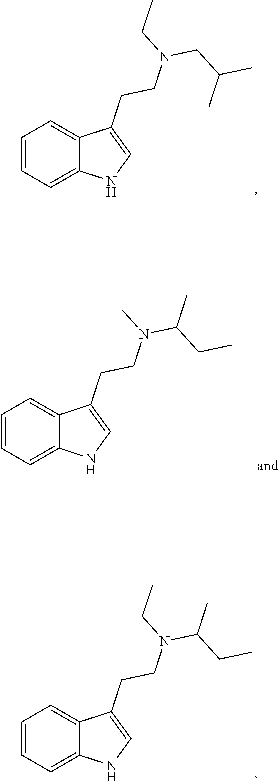 [SMILES: CCN(CCc1c[nH]c2ccccc12)CC(C)C ; CCC(C)N(C)CCc1c[nH]c2ccccc12 ; CCC(C)N(CC)CCc1c[nH]c2ccccc12]

or a pharmaceutically acceptable salt, solvate, tautomer, N-oxide, stereoisomer, metabolite, polymorph or prodrug thereof.

In another aspect of the present disclosure there is provided a method of treating a disease, disorder or condition by activation of a serotonin receptor, the method comprising administering to a subject in need thereof a compound of formula (I) according to any one of the herein disclosed embodiments, or a pharmaceutically acceptable salt, solvate, tautomer, N-oxide, stereoisomer, metabolite, polymorph or prodrug thereof, in combination with another known agent useful for treatment of a disease, disorder or condition by activation of a serotonin receptor.

In another aspect of the present disclosure there is provided a method of treating a mental illness, the method comprising administering to a subject in need thereof a compound of formula (I) according to any one of the herein disclosed embodiments, or a pharmaceutically acceptable salt, solvate, tautomer, N-oxide, stereoisomer, metabolite, polymorph or prodrug thereof.

In embodiments, the mental illness is selected from anxiety disorders; depression; mood disorders; psychotic disorders; impulse control and addiction disorders; drug addiction; obsessive-compulsive disorder (OCD); post-traumatic stress disorder (PTSD); stress response syndromes; dissociative disorders; depersonalization disorder; factitious disorders; sexual and gender disorders; somatic symptom disorders; hallucinations; delusions; psychosis; and combinations thereof.

In another aspect of the present disclosure there is provided a method for treating a central nervous system (CNS) disease, disorder or condition and/or a neurological disease, disorder or condition, the method comprising administering to a subject in need thereof a compound of formula (I) according to any one of the herein disclosed embodiments, or a pharmaceutically acceptable salt, solvate, tautomer, N-oxide, stereoisomer, metabolite, polymorph or prodrug thereof.

In embodiments, the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

In another aspect of the present disclosure there is provided a method for increasing neuronal plasticity and/or increasing dendritic spine density, the method comprising contacting a neuronal cell with a compound of formula (I) according to any one of the herein disclosed embodiments, or a pharmaceutically acceptable salt, solvate, tautomer, N-oxide, stereoisomer, metabolite, polymorph or prodrug thereof, in an amount sufficient to increase neuronal plasticity and/or increase dendritic spine density of the neuronal cell.

In another aspect the present disclosure provides methods of treating weight, comprising administering an effective amount of a compound of the invention to a subject in need thereof. Treatment of weight may include treating weight gain; weight loss; metabolic disorder; weight gain associated with pharmaceutical intervention: weight gain associated with a mental illness (including those described herein); eating disorders such as anorexia, bulimia, cachexia, etc.; eating behaviour; obesity; diabetes; insulin resistance; pre-diabetes; glucose intolerance; hyperlipidemia; and cardiovascular disease.

In another aspect the present disclosure provides a method for activating a serotonin receptor in a cell, either in a biological sample or in a patient, comprising administering a compound of formula (I) as defined in any one of the herein disclosed embodiments to the cell.

Any embodiment herein shall be taken to apply mutatis mutandis to any other embodiment unless specifically stated otherwise.

The present disclosure is not to be limited in scope by the specific embodiments described herein, which are intended for the purpose of exemplification only.

Functionally-equivalent products, compositions and methods are clearly within the scope of the invention, as described herein.

### Definitions

For purposes of interpreting this specification, terms used in the singular will also include the plural and vice versa.

As used herein, except where the context requires otherwise, the term “comprise” and variations of the term, such as “comprising”, “comprises” and “comprised”, are not intended to exclude further additives, components, integers or steps.

The terms “treatment” or “treating” of a subject includes delaying, slowing, stabilizing, curing, healing, alleviating, relieving, altering, remedying, less worsening, ameliorating, improving, or affecting the disease or condition, the sign or symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of signs or symptoms or making the injury, pathology or condition more tolerable to the individual; slowing in the rate of degeneration or decline; making the final point of degeneration less debilitating.

In particularly preferred embodiments, the methods of the present invention can be to prevent or reduce the severity, or inhibit or minimise progression, of a sign or symptom of a disease or condition as described herein. As such, the methods of the present invention have utility as treatments as well as prophylaxes.

As used herein, “preventing” or “prevention” is intended to refer to at least the reduction of likelihood of the risk of (or susceptibility to) acquiring a disease or disorder (i.e., causing at least one of the clinical signs or symptoms of the disease not to develop in an individual that may be exposed to or predisposed to the disease but does not yet experience or display signs or symptoms of the disease). Biological and physiological parameters for identifying such patients are provided herein and are also well known by physicians.

Herein, the term “subject” or “patient” can be used interchangeably with each other. The term “individual” or “patient” refers to an animal that is treatable by the compound and/or method, respectively, including but not limited to, for example, dogs, cats, horses, sheep, pigs, cows, and the like, as well as human, non-human primates. Unless otherwise specified, the “subject” or “patient” may include both male and female genders. Further, it also includes a subject or patient, preferably a human, suitable for receiving treatment with a pharmaceutical composition and/or method of the present invention.

The term “selective” means a greater activity against a first target (e.g., a 5-HT receptor subtype) relative to a second target (e.g., a second 5-HT receptor subtype). In some embodiments a compound has a selectivity of at least 1.25-fold, at least 1.5 fold, at least 2-fold, at least 3-fold, at least 4-fold, at least 5-fold, at least 6-fold, at least 10-fold or at least 100-fold greater towards a first target relative to a second target. In some embodiments, a compound described herein is selective towards the 5-HT_{2A} receptor relative to one or more other 5-HT receptor subtypes such as 5-HT_{2B} and/or 5-HT_{2C}, preferably 5-HT_{2B}. In some embodiments, a compound described herein is selective towards the 5-HT_{2C} receptor relative to one or more other 5-HT receptor subtypes such as 5-HT_{2A} and/or 5-HT_{2B}, preferably 5-HT_{2B}.

“About” as used herein when referring to a measurable value such as an amount, a temporal duration, and the like, is meant to encompass variations of ±20% or ±10%, in some instances ±5%, in some instances 1%, and in some instances ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein the term “alkyl” refers to a straight or branched chain hydrocarbon radical having from one to twelve carbon atoms, or any range between, i.e. it contains 1, 2, 3, 4, 5, 6, 7, 8, 9, 10, 11 or 12 carbon atoms. The alkyl group is optionally substituted with substituents. Examples of “alkyl” as used herein include, but are not limited to, methyl, ethyl, n-propyl, isopropyl, n-butyl, isobutyl, t-butyl, n-pentyl, isopentyl, and the like.

As used herein, the terms “C₁-C₂ alkyl”, “C₁-C₃ alkyl” and “C₁-C₆ alkyl” refer to an alkyl group, as defined herein, containing at least 1, and at most 2, 3 or 6 carbon atoms respectively, or any range in between (eg alkyl groups containing 2-5 carbon atoms are also within the range of C₁-C₆).

The term “alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene.

The term “alkenyl” whether it is used alone or as part of another group, means a straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₂₋₆ alkylene means an alkylene group having 2, 3, 4, 5 or 6 carbon atoms.

Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆ alkynyl means an alkynyl group having 2, 3, 4, 5 or 6 carbon atoms. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl.

The term “cycloalkyl” is intended to include mono-, bi- or tricyclic alkyl groups. The number of carbon atoms that are possible in the referenced cycloalkyl group are indicated by the prefix “Cₙ₁₋ₙ₂”. For example, the term C₃₋₈ cycloalkyl means an cycloalkyl group having 3, 4, 5, 6, 7 or 8 carbon atoms. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “alkylenecycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined herein. The numerical range from x to y in “C_{x-y} alkylenecycloalkyl” relates to the total number of alkyl carbons and cycloalkyl ring atoms. Exemplary alkylenecycloalkyl groups include, but are not limited to, methylenecyclopropyl, methylenecyclobutyl, methylenecyclopentyl and methylenecyclohexyl.

The term “aryl” refers to an aromatic ring system having any suitable number of ring atoms and any suitable number of rings. The number of carbon atoms that are possible in the referenced aryl group are indicated by the prefix “Cₙ₁₋ₙ₂”. For example, the term C₆₋₁₂ aryl means an aryl group having 6, 7, 8, 9, 10, 11 or 12 carbon atoms. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl.

The term “alkylenearyl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂-6, C₃-4, C₃-5, C₃-6, C₄-5, C₄-6 and C₅₋₆. In some instances, the alkyl component can be absent. The aryl component is as defined above. The numerical range from x to y in “C_{x-y} alkylenearyl” relates to the total number of alkyl carbons and aryl ring atoms. Examples of alkylenearyl groups include, but are not limited to, benzyl and ethylenephenyl.

As used herein, the term “alkoxy” refers to an alkyl group as defined herein covalently bound via an O linkage. The alkoxy group is optionally substituted with substituents. Examples of “alkoxy” as used herein include, but are not limited to methoxy, ethoxy, propoxy, isoproxy, butoxy, iso-butoxy, tert-butoxy and pentoxy.

As used herein, the terms “C₁-C₂ alkoxy”, “C₁-C₃ alkoxy” and “C₁-C₆ alkoxy” refer to an alkoxy group, as defined herein, containing at least 1, and at most 2, 3 or 6 carbon atoms respectively, or any range in between (eg alkoxy groups containing 2-5 carbon atoms are also within the range of C₁-C₆).

As used herein, the term “alkylamine” refers to an alkyl group as defined herein having one or more amino groups. The amino groups can be primary, secondary or tertiary. The alkyl amine can be further substituted with a hydroxy group to form an amino-hydroxy group. Examples of alkylamines include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group.

As used herein, the terms “C₁-C₂ alkylamine”, “C₁-C₃ alkylamine” and “C₁-C₆ alkylamine” refer to an alkylamine group, as defined herein, containing at least 1, and at most 2, 3 or 6 carbon atoms respectively, or any range in between (e.g., alkylamine groups containing 2-5 carbon atoms are also within the range of C₁-C₆).

As used herein, the term “alkylsulfonyl” refers to an alkyl group as defined herein having one or more sulfonyl groups. The sulfonyl group can link the alkylsulfonyl to the point of attachment with the rest of the compound, be at the omega position of the alkyl group, or link together at least two carbon atoms of the alkyl group.

As used herein, the terms “C₁-C₂ alkylsulfonyl”, “C₁-C₃ alkylsulfonyl” and “C₁-C₆ alkylsulfonyl” refer to an alkylsulfonyl group, as defined herein, containing at least 1, and at most 2, 3 or 6 carbon atoms respectively, or any range in between (e.g., alkylsulfonyl groups containing 2-5 carbon atoms are also within the range of C₁-C₆).

The term “heteroatom” as used herein means an atom of any element other than carbon or hydrogen. Examples of heteroatoms include nitrogen, oxygen, sulfur and phosphorus. Preferred heteroatoms include N, O and S, preferably N and O.

The term “heteromoiety” as used herein means a chemical group comprising a heteroatom. Examples of heteromoieties include O, S, S(O), SO₂, N and NH.

A “substituent” as used herein, refers to a molecular moiety that is covalently bonded to an atom within a molecule of interest. For example, a “ring substituent” may be a moiety such as a halogen, alkyl group, or other substituent described herein that is covalently bonded to an atom, preferably a carbon or nitrogen atom, that is a ring member. The term “substituted,” as used herein, means that any one or more hydrogens on the designated atom is replaced with a selection from the indicated substituents, provided that the designated atom's normal valence is not exceeded, and that the substitution results in a stable compound, ie, a compound that can be isolated, characterized and tested for biological activity.

The terms “optionally substituted” or “may be substituted” and the like, as used throughout the specification, denotes that the group may or may not be further substituted or fused (so as to form a polycyclic system), with one or more non-hydrogen substituent groups. Suitable chemically viable substituents for a particular functional group will be apparent to those skilled in the art.

Examples of substituents include but are not limited to C₁-C₆ alkyl, C₁-C₆ haloalkyl, C₁-C₆ haloalkoxy, C₁-C₆ hydroxyalkyl, C₃-C₇ heterocyclyl, C₃-C₇ cycloalkyl, C₁-C₆ alkoxy, C₁-C₆ alkylsulfanyl, C₁-C₆ alkylsulfenyl, C₁-C₆ alkylsulfonyl, C₁-C₆ alkylsulfonylamino, arylsulfonoamino, alkylcarboxy, alkylcarboxyamide, oxo, hydroxy, mercapto, amino, acyl, carboxy, carbamoyl, aryl, aryloxy, heteroaryl, aminosulfonyl, aroyl, aroylamino, heteroaroyl, acyloxy, aroyloxy, heteroaroyloxy, alkoxycarbonyl, nitro, cyano, halo, ureido, C₁-C₆ perfluoroalkyl. Preferably the substituents include amino, halo, C₁-C₆ alkyl, amido, hydroxyl.

As used herein, the term “halogen” refers to fluorine (F), chlorine (Cl), bromine (Br), or iodine (I) and the term “halo” refers to the halogen radicals fluoro (—F), chloro (—Cl), bromo (—Br), and iodo (—I). Preferably, ‘halo’ is fluoro or chloro.

As used herein, the term “haloalkyl” refers to an alkyl group as defined herein in which one or more (up to all) of the available hydrogen atoms have been replaced with a halogen. In some instances, the term“perfluoro” can be used to define a compound or radical where all the hydrogens are replaced with fluorine. For example, perfluoromethyl refers to 1,1,1-trifluoromethyl.

As used herein, the terms “C₁-C₂ haloalkyl”, “C₁-C₃ haloalkyl” and “C₁-C₆ haloalkyl” refer to a haloalkyl group, as defined herein, containing at least 1, and at most 2, 3 or 6 carbon atoms respectively, or any range in between (e.g. haloalkyl groups containing 2-5 carbon atoms are also within the range of C₁-C₆).

For example a C₁ haloalkyl group could be, but is not limited to, fluoromethyl, or difluoromethyl, or trifluoromethyl.

As used herein, the term “haloalkenyl” refers to an alkenyl group as defined above in which one or more of the available hydrogen atoms have been replaced with a halogen. Thus, for example, “C₁₋₆ haloalkenyl” (or “C₁-C₆ haloalkenyl”) refers to a C₁ to C₆ linear or branched alkenyl group as defined above with one or more halogen substituents.

As used herein, the term “haloalkynyl” refers to an alkynyl group as defined above in which one or more of the available hydrogen atoms have been replaced with a halogen.

Thus, for example, “C₁₋₆ haloalkynyl” (or “C₁-C₆ haloalkynyl”) refers to a C₁ to C₆ linear or branched alkynyl group as defined above with one or more halogen substituents.

As used herein the term haloalkoxy refers to an alkoxy group as defined herein substituted with at least one halogen.

The term “amino” or “amine” refers to the group —NH₂.

The term “substituted amino” or “secondary amino” refers to an amino group having a hydrogen replaced with, for example a C₁-C₆ alkyl group (“C₁-C₆ alkylamino”), an aryl or aralkyl group (“arylamino”, “aralkylamino”) and so on. C₁-C₃ alkylamino groups are preferred, such as for example, methylamino (NHMe), ethylamino (NHEt) and propylamino (NHPr).

The term “disubstituted amino” or “tertiary amino” refers to an amino group having the two hydrogens replaced with, for example a C₁-C₆alkyl group, which may be the same or different (“dialkylamino”), an aryl and alkyl group (“aryl(alkyl)amino”) and so on. Di(C₁-C₃alkyl)amino groups are preferred, such as for example, dimethylamino (NMe₂), diethylamino (NEt₂), dipropylamino (NPr₂) and variations thereof (eg N(Me)(Et) and so on).

The term “nitro” refers to the group —NO₂.

The term “cyano” and “nitrile” refer to the group —CN.

The term “amido” or “amide” refers to the group —C(O)NH₂.

The term “substituted amido” or “substituted amide” refers to an amido group having a hydrogen replaced with, for example a C₁-C₆ alkyl group (“C₁-C₆ alkylamido” or “C₁-C₆ alkylamide”), an aryl (“arylamido”), aralkyl group (“aralkylamido”) and so on. C₁-C₃ alkylamide groups are preferred, such as for example, methylamide (—C(O)NHMe), ethylamide (—C(O)NHEt) and propylamide (—C(O)NHPr) and includes reverse amides thereof (eg NHMeC(O)—, —NHEtC(O)— and —NHPrC(O)—).

The term “disubstituted amido” or “disubstituted amide” refers to an amido group having the two hydrogens replaced with, for example a C₁-C₆alkyl group (“di(C₁-C₆ alkyl)amido” or “di(C₁-C₆ alkyl)amide”), an aralkyl and alkyl group (“alkyl(aralkyl)amido”) and so on. Di(C₁-C₃ alkyl)amide groups are preferred, such as for example, dimethylamide (—C(O)NMe₂), diethylamide (—C(O)NEt₂) and dipropylamide ((—C(O)NPr₂) and variations thereof (eg C(O)N(Me)Et and so on) and includes reverse amides thereof.

The term “sulfonyl” refers to the group —SO₂H.

The term “substituted sulfonyl” refers to a sulfonyl group having the hydrogen replaced with, for example a C₁-C₆ alkyl group (“sulfonylC₁-C₆ alkyl”), an aryl (“arylsulfonyl”), an aralkyl (“aralkylsulfonyl”) and so on. Sulfonyl C₁-C₃ alkyl groups are preferred, such as for example, —SO₂Me, —SO₂Et and —SO₂Pr.

The term “sulfonylamido” or “sulfonamide” refers to the group —SO₂NH₂.

The term “substituted sulfonamido” or “substituted sulphonamide” refers to an sulfonylamido group having a hydrogen replaced with, for example a C₁-C₆ alkyl group (“sulfonylamidoC₁-C₆ alkyl”), an aryl (“arylsulfonamide”), aralkyl (“aralkylsulfonamide”) and so on. SulfonylamidoC₁-C₃ alkyl groups are preferred, such as for example, SO₂NHMe, SO₂NHEt and —SO₂NHPr and includes reverse sulfonamides thereof (e.g. —NHSO₂Me, NHSO₂Et and —NHSO₂Pr).

The term “disubstituted sufonamido” or “disubstituted sulphonamide” refers to an sulfonylamido group having the two hydrogens replaced with, for example a C₁-C₆ alkyl group, which may be the same or different (“sulfonylamidodi(C₁-C₆ alkyl)”), an aralkyl and alkyl group (“sulfonamido(aralkyl)alkyl”) and so on. Sulfonylamidodi(C₁-C₃ alkyl) groups are preferred, such as for example, —SO₂NMe₂, —SO₂NEt₂ and —SO₂NPr₂ and variations thereof (eg SO₂N(Me)Et and so on) and includes reserve sulfonamides thereof (eg —N(Me)SO₂Me and so on).

The term “sulfate” refers to the group OS(O)₂OH and includes groups having the hydrogen replaced with, for example a C₁-C₆ alkyl group (“alkylsulfates”), an aryl (“arylsulfate”), an aralkyl (“aralkylsulfate”) and so on. C₁-C₃ alkylsulfates are preferred, such as for example, OS(O)₂OMe, OS(O)₂OEt and OS(O)₂OPr.

The term “sulfonate” refers to the group SO₃H and includes groups having the hydrogen replaced with, for example a C₁-C₆ alkyl group (“alkylsulfonate”), an aryl (“arylsulfonate”), an aralkyl (“aralkylsulfonate”) and so on. C₁-C₃ alkylsulfonates are preferred, such as for example, SO₃Me, SO₃Et and SO₃Pr.

The term “amino acid” as herein defined refers to a moiety containing an amino group and a carboxyl group linked by at least one carbon. An amino acid may refer a natural or non-natural amino acid, preferably a natural amino acid such as alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, preferably the amino acid is arginine, lysine or histidine, most preferably lysine.

The term “carboxylate” or “carboxyl” refers to the group —COO— or —COOH.

The term “carbamate” or “carbomyl” refers to the group —OC(O)NH₂. The carbamate may be substituted, or may be disubstituted, for example with an alkyl group such as but not limited to C₁-C₆ alkyl.

The term “carbonate” refers to the group —OC(O)O— or —OC(O)OH.

The term “alkylcarbonate” as herein defined refers to a carbonate group having the hydrogen replaced with, for example a C₁-C₆ alkyl group, an aryl or aralkyl group (“arylcarbonate” or “aralkylcarbonate”) and so on. CO₃C₁-C₃alkyl groups are preferred, such as for example, methylcarbonate (CO₃Me), ethylcarbonate (CO₃Et) and propylcarbonate (CO₃Pr).

The term “ester” refers to a carboxyl group having the hydrogen replaced with, for example a C₁-C₆ alkyl group (“carboxylC₁-C₆ alkyl” or “alkylester”), an aryl or aralkyl group (“arylester” or “aralkylester”) and so on. CO₂C₁-C₃ alkyl groups are preferred, such as for example, methylester (CO₂Me), ethylester (CO₂Et) and propylester (CO₂Pr) and includes reverse esters thereof (eg —OC(O)Me, —OC(O)Et and —OC(O)Pr).

The term “heterocyclyl” refers to a moiety obtained by removing a hydrogen atom from a ring atom of a heterocyclic compound which moiety has from 3 to 12 ring atoms (unless otherwise specified), of which 1, 2, 3, 4 or more are ring heteroatoms, for example independently selected from O, S and N, or ring heteromoieties, for example independently selected from O, S, S(O), SO₂, N and NH. When a heterocyclyl group contains the prefix Cₙ₁₋ₙ₂ or “n1 to n2” this prefix indicates the number of carbon atoms in the corresponding carbocyclic group, in which one or more, suitably 1, 2, 3, 4 or more, of the ring atoms is replaced with a heteroatom or heteromoiety.

In this context, the prefixs 3-, 4-, 5-, 6-, 7-, 8-, 9- and 10-membered denote the number of ring atoms, or range of ring atoms, whether carbon atoms or heteroatoms. For example, the term “C₃₋₁₀ heterocyclyl” or “3-10 membered heterocylyl”, as used herein, pertains to a heterocyclyl group having 3, 4, 5, 6, 7, 8, 9 or 10 ring atoms. Examples of heterocylyl groups include 5-6-membered monocyclic heterocyclyls and 9-10 membered fused bicyclic heterocyclyls.

Examples of monocyclic heterocyclyl groups include, but are not limited to, those containing one nitrogen atom such as aziridine (3-membered ring), azetidine (4-membered ring), pyrrolidine (tetrahydropyrrole), pyrroline (eg 3-pyrroline, 2,5-dihydropyrrole), 2Hpyrrole or 3H-pyrrole (isopyrrole, isoazole) or pyrrolidinone (5-membered rings), piperidine, dihydropyridine, tetrahydropyridine (6-membered rings), and azepine (7membered ring); those containing two nitrogen atoms such as imidazoline, pyrazolidine (diazolidine), imidazoline, pyrazoline (dihydropyrazole) (5-membered rings), piperazine (6membered ring); those containing one oxygen atom such as oxirane (3-membered ring), oxetane (4-membered ring), oxolane (tetrahydrofuran), oxole (dihydrofuran) (5-membered rings), oxane (tetrahydropyran), dihydropyran, pyran (6-membered rings), oxepin (7membered ring); those containing two oxygen atoms such as dioxolane (5-membered ring), dioxane (6-membered ring), and dioxepane (7-membered ring); those containing three oxygen atoms such as trioxane (6-membered ring); those containing one sulfur atom such as thiirane (3-membered ring), thietane (4-membered ring), thiolane (tetrahydrothiophene) (5-membered ring), thiane (tetrahydrothiopyran) (6-membered ring), thiepane (7-membered ring); those containing one nitrogen and one oxygen atom such as tetrahydrooxazole, dihydrooxazole, tetrahydroisoxazole, dihydroisoxazole (5-membered rings), morpholine, tetrahydrooxazine, dihydrooxazine, oxazine (6-membered rings); those containing one nitrogen and one sulfur atom such as thiazoline, thiazolidine (5-membered rings), thiomorpholine (6-membered ring); those containing two nitrogen and one oxygen atom such as oxadiazine (6-membered ring); those containing one oxygen and one sulfur such as: oxathiole (5-membered ring) and oxathiane (thioxane) (6-membered ring); and those containing one nitrogen, one oxygen and one sulfur atom such as oxathiazine (6-membered ring).

Heterocyclyls also encompass heteroaryl (aromatic heterocyclyls) and heterocycloalkyl (non-aromatic heterocyclyls). Such groups may be substituted or unsubstituted.

The term “aromatic heterocyclyl” may be used interchangeably with the term “heteroaromatic” or the term “heteroaryl” or “hetaryl”. The heteroatoms in the aromatic heterocyclyl group may be independently selected from N, S and O. The aromatic heterocyclyl groups may comprise 1, 2, 3, 4 or more ring heteroatoms. When a heteroaryl group contains the prefix Cₙ₁₋ₙ₂ or “n1 to n2” this prefix indicates the number of carbon atoms in the corresponding aryl group, in which one or more, suitably 1, 2, 3, 4 or more, of the ring atoms is replaced with a heteroatom. In the case of fused aromatic heterocyclyl groups, only one of the rings may contain a heteroatom and not all rings must be aromatic.

“Heteroaryl” is used herein to denote a heterocyclic group having aromatic character and embraces aromatic monocyclic ring systems and polycyclic (eg bicyclic) ring systems containing one or more aromatic rings. The term aromatic heterocyclyl also encompasses pseudoaromatic heterocyclyls. The term “pseudoaromatic” refers to a ring system which is not strictly aromatic, but which is stabilized by means of delocalization of electrons and behaves in a similar manner to aromatic rings. The term aromatic heterocyclyl therefore covers polycyclic ring systems in which all of the fused rings are aromatic as well as ring systems where one or more rings are non-aromatic, provided that at least one ring is aromatic. In polycyclic systems containing both aromatic and non-aromatic rings fused together, the group may be attached to another moiety by the aromatic ring or by a non-aromatic ring.

Examples of heteroaryl groups are monocyclic and bicyclic groups containing from five to ten ring members. The heteroaryl group can be, for example, a five membered or six membered monocyclic ring or a bicyclic structure formed from fused five and six membered rings or two fused six membered rings or two fused five membered rings. Each ring may contain up to about four heteroatoms typically selected from nitrogen, sulphur and oxygen. The heteroaryl ring will contain up to 4 heteroatoms, more typically up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Aromatic heterocyclyl groups may be 5-membered or 6-membered mono-cyclic aromatic ring systems.

Examples of 5-membered monocyclic heteroaryl groups include but are not limited to furanyl, thienyl, pyrrolyl, oxazolyl, oxadiazolyl (including 1,2,3 and 1,2,4 oxadiazolyls and furazanyl i.e. 1,2,5-oxadiazolyl), thiazolyl, isoxazolyl, isothiazolyl, pyrazolyl, imidazolyl, triazolyl (including 1,2,3, 1,2,4 and 1,3,4 triazolyls), oxatriazolyl, tetrazolyl, thiadiazolyl (including 1,2,3 and 1,3,4 thiadiazolyls) and the like.

Examples of 6-membered monocyclic heteroaryl groups include but are not limited to pyridinyl, pyrimidinyl, pyridazinyl, pyrazinyl, triazinyl, pyranyl, oxazinyl, dioxinyl, thiazinyl, thiadiazinyl and the like. Examples of 6-membered aromatic heterocyclyls containing nitrogen include pyridyl (1 nitrogen), pyrazinyl, pyrimidinyl and pyridazinyl (2 nitrogens).

Aromatic heterocyclyl groups may also be bicyclic or polycyclic heteroaromatic ring systems such as fused ring systems (including purine, pteridinyl, napthyridinyl, 1H thieno[2,3-c]pyrazolyl, thieno[2,3-b]furyl and the like) or linked ring systems (such as oligothiophene, polypyrrole and the like). Fused ring systems may also include aromatic 5membered or 6-membered heterocyclyls fused to carbocyclic aromatic rings such as phenyl, napthyl, indenyl, azulenyl, fluorenyl, anthracenyl and the like, such as 5-membered aromatic heterocyclyls containing nitrogen fused to phenyl rings, 5-membered aromatic heterocyclyls containing 1 or 2 nitrogens fused to phenyl ring.

A bicyclic heteroaryl group may be, for example, a group selected from: a) a benzene ring fused to a 5- or 6-membered ring containing 1, 2 or 3 ring heteroatoms; b) a pyridine ring fused to a 5- or 6-membered ring containing 1, 2 or 3 ring heteroatoms; c) a pyrimidine ring fused to a 5- or 6-membered ring containing 1 or 2 ring heteroatoms; d) a pyrrole ring fused to a 5- or 6-membered ring containing 1, 2 or 3 ring heteroatoms; e) a pyrazole ring fused to a 5- or 6-membered ring containing 1 or 2 ring heteroatoms; f) an imidazole ring fused to a 5- or 6-membered ring containing 1 or 2 ring heteroatoms; g) an oxazole ring fused to a 5- or 6-membered ring containing 1 or 2 ring heteroatoms; h) an isoxazole ring fused to a 5- or 6-membered ring containing 1 or 2 ring heteroatoms; i) a thiazole ring fused to a 5- or 6-membered ring containing 1 or 2 ring heteroatoms; j) an isothiazole ring fused to a 5- or 6-membered ring containing 1 or 2 ring heteroatoms; k) a thiophene ring fused to a 5- or 6-membered ring containing 1, 2 or 3 ring heteroatoms; l) a furan ring fused to a 5- or 6membered ring containing 1, 2 or 3 ring heteroatoms; m) a cyclohexyl ring fused to a 5- or 6-membered ring containing 1, 2 or 3 ring heteroatoms; and n) a cyclopentyl ring fused to a 5- or 6-membered ring containing 1, 2 or 3 ring heteroatoms.

Particular examples of bicyclic heteroaryl groups containing a five membered ring fused to another five membered ring include but are not limited to imidazothiazole (e.g. imidazo[2,1-b]thiazole) and imidazoimidazole (e.g. imidazo[1,2-a]imidazole).

Particular examples of bicyclic heteroaryl groups containing a six membered ring fused to a five membered ring include but are not limited to benzofuran, benzothiophene, benzimidazole, benzoxazole, isobenzoxazole, benzisoxazole, benzothiazole, benzisothiazole, isobenzofuran, indole, isoindole, indolizine, indoline, isoindoline, purine (e.g., adenine, guanine), indazole, pyrazolopyrimidine (e.g. pyrazolo[1,5-a]pyrimidine), benzodioxole and pyrazolopyridine (e.g. pyrazolo[1,5-a]pyridine) groups. A further example of a six membered ring fused to a five membered ring is a pyrrolopyridine group such as a pyrrolo[2,3-b]pyridine group.

Particular examples of bicyclic heteroaryl groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

Examples of heteroaryl groups containing an aromatic ring and a non-aromatic ring include tetrahydronaphthalene, tetrahydroisoquinoline, tetrahydroquinoline, dihydrobenzothiophene, dihydrobenzofuran, 2,3-dihydro-benzo[1,4]dioxine, benzo[1,3]dioxole, 4,5,6,7-tetrahydrobenzofuran, indoiine, isoindoline and indane groups.

Examples of aromatic heterocyclyls fused to carbocyclic aromatic rings may therefore include but are not limited to benzothiophenyl, indolyl, isoindolyl, benzofuranyl, isobenzofuranyl, benzimidazolyl, indazolyl, benzoxazolyl, benzisoxazolyl, isobenzoxazoyl, benzothiazolyl, benzisothiazolyl, quinolinyl, isoquinolinyl, quinoxalinyl, quinazolinyl, cinnolinyl, benzotriazinyl, phthalazinyl, carbolinyl and the like.

The term “heterocycloalkyl” or “non-aromatic heterocyclyl” encompasses optionally substituted saturated and unsaturated rings which contain at least one heteroatom such as N, S and O, or a heteromoiety such as O, S, S(O), SO₂, N and NH. The ring may contain 1, 2, 3, 4 or more heteroatoms or heteromoieties. When a heterocycloalkyl group contains the prefix Cₙ₁₋ₙ₂ or “n1 to n2” this prefix indicates the number of carbon atoms in the corresponding carbocyclic group, in which one or more, suitably 1, 2, 3, 4 or more, of the ring atoms is replaced with a heteroatom or heteromoiety. The ring may be a monocyclic ring or part of a polycyclic ring system. Polycyclic ring systems include fused rings and spirocycles. Not every ring in a non-aromatic heterocyclic polycyclic ring system must contain a heteroatom, provided at least one ring contains one or more heteroatoms.

Non-aromatic heterocyclyls may be 3-8 membered mono-cyclic rings.

Examples of 5-membered non-aromatic heterocyclyl rings include 2H-pyrrolyl, 1pyrrolinyl, 2-pyrrolinyl, 3-pyrrolinyl, pyrrolidinyl, 1-pyrrolidinyl, 2-pyrrolidinyl, 3-pyrrolidinyl, tetrahydrofuranyl, tetrahydrothiophenyl, pyrazolinyl, 2-pyrazolinyl, 3-pyrazolinyl, pyrazolidinyl, 2-pyrazolidinyl, 3-pyrazolidinyl, imidazolidinyl, 3-dioxalanyl, thiazolidinyl, isoxazolidinyl, 2-imidazolinyl and the like.

Examples of 6-membered non-aromatic heterocyclyls include piperidinyl, piperidinonyl, pyranyl, dihyrdopyranyl, tetrahydropyranyl, 2H pyranyl, 4H pyranyl, thianyl, thianyl oxide, thianyl dioxide, piperazinyl, diozanyl, 1,4-dioxinyl, 1,4-dithianyl, 1,3,5triozalanyl, 1,3,5-trithianyl, 1,4-morpholinyl, thiomorpholinyl, 1,4-oxathianyl, triazinyl, 1,4thiazinyl and the like.

Examples of 7-membered non-aromatic heterocyclyls include azepanyl, oxepanyl, thiepanyl and the like.

Non-aromatic heterocyclyl rings may also be bicyclic heterocyclyl rings such as linked ring systems (for example uridinyl and the like) or fused ring systems. Fused ring systems include non-aromatic 5-membered, 6-membered or 7-membered heterocyclyls fused to carbocyclic aromatic rings such as phenyl, napthyl, indenyl, azulenyl, fluorenyl, anthracenyl and the like. Examples of non-aromatic 5-membered, 6-membered or 7membered heterocyclyls fused to carbocyclic aromatic rings include indolinyl, benzodiazepinyl, benzazepinyl, dihydrobenzofuranyl and the like.

The term “alkyleneheteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The heteroaryl component is as defined herein. The numerical range from x to y in “C_{x-y} alkyleneheteroaryl” relates to the total number of alkyl carbons and heteroaryl ring atoms (carbon and heteroatoms together.

The term “alkyleneheterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The heterocycloalkyl component is as defined herein. The numerical range from x to y in “C_{x-y} alkyleneheterocycloalkyl” relates to the total number of alkyl carbons and heterocycloalkyl ring atoms (carbon and heteroatoms together).

As used herein, the term solvate refers to a complex of the compound and either stoichiometric or non-stoichiometric amounts of a solvent. Solvates are often formed during the process of crystallization with pharmaceutically acceptable solvents such as water, ethanol, and the like. Hydrates are formed when the solvent is water, or alcoholates are formed when the solvent is alcohol.

As used herein, the term polymorph refers to the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

As used herein, the term “metabolite” refers to a derivative of a compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. Metabolites of the compounds disclosed herein are optionally identified either by administration of compounds to a host and analysis of tissue samples from the host, or by incubation of compounds with hepatic cells in vitro and analysis of the resulting compounds.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the invention may contain asymmetric or chiral centers, and therefore exist in different stereoisomeric forms. The term “stereoisomers” refers to compounds which have identical chemical constitution, but differ with regard to the arrangement of the atoms or groups in space. As used herein, the term “stereoisomer” includes but is not limited to diastereomers, enantiomers and atropisomers, as well as mixtures thereof such as racemic mixtures.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

### Forms of the Compound

In the case of compounds that are solids, it will be understood by those skilled in the art that the inventive compounds, agents and salts may exist in different crystalline or polymorphic forms, all of which are intended to be within the scope of the present invention and specified formulae.

The invention includes all crystalline forms of a compound of Formula (I) including anhydrous crystalline forms, hydrates, solvates and mixed solvates. If any of these crystalline forms demonstrates polymorphism, all polymorphs are within the scope of this invention.

Formula (I) is intended to cover, where applicable, solvated as well as unsolvated forms of the compounds. Thus, Formula (I) includes compounds having the indicated structures, including the hydrated or solvated forms, as well as the non-hydrated and non-solvated forms.

The compounds of Formula (I) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF), acetic acid, and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the invention.

Basic nitrogen-containing groups may be quarternised with such agents as C₁₋₆alkyl halide, such as methyl, ethyl, propyl, and butyl chlorides, bromides and iodides; dialkyl sulfates like dimethyl and diethyl sulfate; and others.

Nitrogen containing groups may also be oxidised to form an N-oxide.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs are within the scope of the invention.

The compound of Formula (I) may demonstrate tautomerism. Tautomers are two interchangeable forms of a molecule that typically exist within an equilibrium. Any tautomers of the compounds of Formula (I) are to be understood as being within the scope of the invention.

The compound of Formula (I) may contain one or more stereocentres. All stereoisomers of the compounds of formula (I) are within the scope of the invention. Stereoisomers include enantiomers, diastereomers, geometric isomers (E and Z olephinic forms and cis and trans substitution patterns) and atropisomers. In some embodiments, the compound is a stereoisomerically enriched form of the compound of formula (I) at any stereocentre. The compound may be enriched in one stereoisomer over another by at least about 60, 70, 80, 90, 95, 98 or 99%.

The compound of Formula (I) or its salts, tautomers, solvates, N-oxides, and/or stereoisomers, may be isotopically enriched with one or more of the isotopes of the atoms present in the compound. For example, the compound may be enriched with one or more of the following minor isotopes: ²H, ³H, ¹³C, ¹⁴C, ¹⁵N and/or ¹⁷O, preferably ²H. An isotope may be considered enriched when its abundance is greater than its natural abundance.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) through the carbonyl carbon prodrug sidechain.

### Compositions, Formulations and Modes of Administration

The compounds of formula (I) can be administered alone or in the form of a pharmaceutical composition. In practice, the compounds of formula (I) are usually administered in the form of pharmaceutical compositions, that is, in admixture with at least one pharmaceutically acceptable excipient. The proportion and nature of any pharmaceutically acceptable excipient(s) are determined by the properties of the selected compound of the invention, the chosen route of administration, and standard pharmaceutical practice.

In another embodiment, there is provided a pharmaceutical composition comprising a compound of formula (I) or a pharmaceutically acceptable salt, stereoisomer, solvate, metabolite, or polymorph thereof, and at least one pharmaceutically acceptable excipient.

Pharmaceutical compositions of the disclosure typically include a therapeutically effective amount of one or more active ingredients in admixture with one or more pharmaceutically and physiologically acceptable formulation materials. Suitable formulation materials include, but are not limited to, antioxidants, preservatives, coloring, flavoring and diluting agents, emulsifying agents, suspending agents, solvents, fillers, bulking agents, buffers, delivery vehicles, diluents, excipients and/or pharmaceutical adjuvants. For example, a suitable vehicle may be water for injection, physiological saline solution, or artificial perilymph, possibly supplemented with other materials common in compositions for parenteral administration. Neutral buffered saline or saline mixed with serum albumin are further exemplary vehicles.

Pharmaceutical compositions of the present disclosure additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this disclosure. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminium hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as colouring agents, releasing agents, coating agents, sweetening, flavouring and perfuming agents, preservatives and antioxidants can also be present in the composition, according to the judgment of the formulator.

Various dosage units are each preferably provided as a discrete dosage tablet, capsules, lozenge, dragee, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

A compound of formula (I) may be administered in any form and route which makes the compound bioavailable.

Compositions described herein may be administered systemically or directly to the site of condition or disease.

Compositions described herein may be formulated from compounds according to Formula (I) for any appropriate route of administration including, for example, oral, rectal, nasal, vaginal, topical (including transdermal, buccal, ocular and sublingual), parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), inhalation, insufflation, infusion or implantation techniques (e.g., as sterile injectable aqueous or non-aqueous solutions or suspensions). In some embodiments, compositions described herein may be administered orally, nasally, intravenously, intramuscularly, topically, subcutaneously, rectally, vaginally or by urethral application.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Oily suspensions may be formulated by suspending the active ingredients in a vegetable oil such as Arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or Arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such Formulations may also comprise one or more demulcents, preservatives, flavouring agents and/or colouring agents.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical Formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

A pharmaceutical composition may be formulated as inhaled formulations, including sprays, mists, or aerosols. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable Formulations, wherein the carrier is a liquid, for administration, as for example, a nasal spray or as nasal drops, include aqueous or oily solutions of the active ingredient.

Compositions suitable for rectal administration are preferably presented as unit dose suppositories. These may be prepared by at least partially dispersing the active in one or more lipophilic bases and then shaping the mixture.

Pharmaceutical compositions may be formulated as sustained release formulations such as a capsule that creates a slow release of active following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of active release. The amount of active contained within a sustained release formulation depends upon, for example, the site of implantation, the rate and expected duration of release and the nature of the condition to be treated.

One skilled in the art can readily select the proper form and route of administration depending on the particular characteristics of the compound selected, the disease or condition to be treated, the stage of the disease or condition, and other relevant circumstances.

It will be understood, that the specific dose level for any particular patient will depend upon a variety of factors including the activity of the specific compound employed, the age, body weight, general health, sex, diet, time of administration, route of administration, number of doses, and rate of excretion, drug combination (i.e. other drugs being used to treat the patient), and the severity of the particular disorder undergoing therapy.

The phrase “therapeutically effective amount” generally refers to an amount of one or more active ingredients of the invention that (i) treats the particular disease, condition, or disorder, (ii) attenuates, ameliorates, or eliminates one or more sign or symptoms of the particular disease, condition, or disorder, or (iii) delays the onset of one or more sign or symptoms of the particular disease, condition, or disorder described herein.

Typically, a therapeutically effective dosage is formulated to contain a concentration (by weight) of at least about 0.1% up to about 50% or more, and all combinations and sub-combinations of ranges therein. The compositions can be formulated to contain one or more actives described herein in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue.

The frequency of administration may be once daily, 2, 3 or 4 times daily. The treatment period may be for the duration of the detectable disease.

In some embodiments, the pharmaceutical composition comprises a compound according to any one of the herein disclosed embodiments, or a pharmaceutically acceptable salt, solvate, tautomer, N-oxide, stereoisomer, metabolite, polymorph or prodrug thereof, an additional therapeutic agent, and a pharmaceutically acceptable excipient.

The additional agent may be any suitable agent described herein. In some embodiments, the additional agent is a psychoactive drug, including those described herein. In some embodiments, the additional agent is useful for treatment of a disease, disorder or condition by activation of a serotonin receptor, including those described herein. In some embodiments, the additional agent is selected from any one of the following, including those described herein: an agent for a mental illness and/or a neuropsychiatric condition; an agent for psychosis and/or psychotic symptoms; an agent for attention deficit hyperactivity disorder and/or attention deficit disorder; an agent for dementia and/or Alzheimer's disease; and an agent for an addiction disorder.

### Applications

The present disclosure provides methods of using the compounds of formula (I) and compositions as described in any one of the foregoing paragraphs. The present disclosure also provides methods of delivering to a subject in need thereof a compound of formula (I) or a composition (e.g., an effective amount of the compound or composition) of the present disclosure.

In another aspect, the present disclosure provides methods of treating a disease in a subject in need thereof comprising administering to the subject in need thereof an effective amount (e.g., therapeutically effective amount) of a compound or composition (e.g., pharmaceutical composition) of the present disclosure.

In another aspect, the present disclosure provides methods of preventing a disease in a subject in need thereof comprising administering to the subject in need thereof an effective amount (e.g., therapeutically effective amount) of a compound of formula (I) or composition (e.g., pharmaceutical composition) of the present disclosure.

In another aspect, provided herein are uses of the compounds of formula (I) or compositions of the present disclosure in the manufacture of a medicament for use in a method (e.g., method of delivering an active agent to a subject in need thereof, method of treating a disease in a subject in need thereof, method of preventing a disease in a subject in need thereof) of the present disclosure.

In another aspect, provided herein are uses of the compounds of formula (I) or compositions of the present disclosure in a method (e.g., method of delivering an active agent to a subject in need thereof, method of treating a disease in a subject in need thereof, method of preventing a disease in a subject in need thereof) of the present disclosure.

In certain embodiments, the effective amount is effective in treating the disease. In certain embodiments, the effective amount is effective in preventing the disease.

In another aspect, the present disclosure provides a method of treating a disease, disorder or condition by activation of a serotonin receptor, the method comprising administering to a subject in need thereof a compound of formula (I) or a pharmaceutical composition as described herein.

In another aspect, the present disclosure provides a method of preventing a disease, disorder or condition by activation of a serotonin receptor, the method comprising administering to a subject in need thereof a compound of formula (I) or a pharmaceutical composition as described herein.

In another aspect, the present disclosure provides method of treating a disease, disorder or condition by activation of a serotonin receptor, the method comprising administering to a subject in need thereof a compound of formula (I) or a pharmaceutical composition as described herein, in combination with another known agent useful for treatment of a disease, disorder or condition by activation of a serotonin receptor. The other known agents useful for treatment of a disease, disorder or condition by activation of a serotonin receptor may be any suitable agents known in the art, including those described herein.

In another aspect, the present disclosure provides method of preventing a disease, disorder or condition by activation of a serotonin receptor, the method comprising administering to a subject in need thereof a compound of formula (I) or a pharmaceutical composition as described herein, in combination with another known agent useful for prevention of a disease, disorder or condition by activation of a serotonin receptor.

In certain embodiments, the serotonin receptor is 5-HT_{2A}.

In certain embodiments, the serotonin receptor is one or both of 5-HT_{2A} and 5-HT_{2C}.

Additionally, or alternatively, in some embodiments, the serotonin receptor is not 5-HT_{2B}.

In some embodiments, the compound of formula (I) of the present disclosure is selective towards the 5-HT_{2A} receptor over one or both of the 5-HT_{2C} receptor and the 5-HT_{2B} receptor, preferably over the 5-HT_{2B} receptor. In some embodiments, the compound of formula (I) is selective towards the 5-HT_{2C} receptor over one or both of the 5-HT_{2A} receptor and the 5-HT_{2B} receptor, preferably over the 5-HT_{2B} receptor. In some embodiments, the compound of formula (I) is selective toward the 5-HT_{2A} receptor and 5-HT_{2C} receptor over the 5-HT_{2B} receptor.

In some embodiments, the compound of formula (I) of the present disclosure exhibits an EC₅₀ value for the 5-HT_{2A} receptor of less than about 1 mM, less than about 100 μM, less than about 10 μM, less than about 1 μM, or less than about 100 nM, or less than about 10 nM, as determined by an assay described herein, for example an assay of calcium flux activity such as measuring changes in intracellular calcium. In some embodiments, the compound of formula (I) exhibits an EC₅₀ for the 5-HT_{2A} receptor of less than about 1 mM, less than about 900 μM, less than about 800 μM, less than about 700 μM, less than about 600 μM, less than about 500 μM, less than about 400 μM, less than about 300 μM, less than about 200 μM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 9 μM, less than about 8 μM, less than about 7 μM, less than about 6 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, or less than about 100 nM, or any equivalent unit of measure (e.g., mol/L), as determined by an assay of calcium flux activity.

In some embodiments, the compound of formula (I) of the present disclosure exhibits an EC₅₀ value for the 5-HT_{2C} receptor of less than about 1 mM, less than about 100 μM, less than about 10 μM, less than about 1 μM, or less than about 100 nM, or less than about 10 nM, as determined by an assay described herein, for example an assay of calcium flux activity such as measuring changes in intracellular calcium. In some embodiments, the compound of formula (I) exhibits an EC₅₀ for the 5-HT_{2C} receptor of less than about 1 mM, less than about 900 μM, less than about 800 μM, less than about 700 μM, less than about 600 μM, less than about 500 μM, less than about 400 μM, less than about 300 μM, less than about 200 μM, less than about 100 μM, less than about 90 μM, less than about 80 μM, less than about 70 μM, less than about 60 μM, less than about 50 μM, less than about 40 μM, less than about 30 μM, less than about 20 μM, less than about 10 μM, less than about 9 μM, less than about 8 μM, less than about 7 μM, less than about 6 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, or less than about 100 nM, or any equivalent unit of measure (e.g., mol/L), as determined by an assay of calcium flux activity.

In some embodiments, the compound of formula (I) of the present disclosure exhibits an EC₅₀ value for the 5-HT_{2B} receptor of greater than about 1 μM, greater than about 10 μM, or greater than about 100 μM, as determined by an assay described herein, for example an assay of calcium flux activity such as measuring changes in intracellular calcium.

In some embodiments, the disease, disorder or condition that is treated by activation of a serotonin receptor is a mental illness or a neuropsychiatric condition. Accordingly, the present application also includes a method of treating a mental illness or a neuropsychiatric condition comprising administering to a subject in need thereof a compound of formula (I) or a composition as described herein. The present application also includes a use of a compound of formula (I) of the present disclosure for treatment of a mental illness or a neuropsychiatric condition, as well as a use of a compound of formula (I) of the present disclosure for the preparation of a medicament for treatment of a mental illness or a neuropsychiatric condition. The application further includes a compound of formula (I) of the present disclosure for use in treating a mental illness or a neuropsychiatric condition.

In some embodiments, the disease, disorder or condition that is treated by activation of a serotonin receptor is a mental illness or a neuropsychiatric condition and compound of formula (I) of the present disclosure is administered in combination with one or more additional agents for a mental illness or a neuropsychiatric condition. The one or more additional agents for a mental illness or a neuropsychiatric condition may be any suitable agents known in the art, including those described herein. In some embodiments, the additional agents for a mental illness or a neuropsychiatric condition is selected from antipsychotics, including typical antipsychotics and atypical antipsychotics; antidepressants including selective serotonin reuptake inhibitors (SSRIs) and selective norepinephrine reuptake inhibitors (SNRIs), tricyclic antidepressants and monoamine oxidase inhibitors (MAOIs) (e.g. bupropion); anti-anxiety medication including benzodiazepines such as alprazolam; agents for an addiction disorder such as alcohol addiction (e.g., disulfiram), nicotine dependence (e.g., varenicline) and opioid use disorder (e.g., methadone, buprenorphine, buprenorphine-naloxone and buprenorphine long-acting injection); mood stabilizers such as lithium and anticonvulsants such carbamazepine, divalproex (valproic acid), lamotrigine, gabapentin and topiramate.

In some embodiments, the disease, disorder or condition that is treated by activation of a serotonin receptor is neurodegeneration. Accordingly, the present application also includes a method of treating neurodegeneration comprising administering to a subject in need thereof a compound of formula (I) or a composition as described herein. The present application also includes a use of a compound of formula (I) of the present disclosure for treatment of neurodegeneration, as well as a use of a compound of formula (I) of the present disclosure for the preparation of a medicament for treatment neurodegeneration. The application further includes a compound of formula (I) of the present disclosure for use in treating neurodegeneration. In some embodiments, the disease, disorder or condition that is treated by activation of a serotonin receptor is reduced brain-derived neurotrophic factor (BDNF), mammalian target of rapamycin (mTOR) activation and/or inflammation.

In some embodiments, the disease, disorder or condition that is treated by activation of a serotonin receptor comprises cognitive impairment; ischemia including stroke; neurodegeneration; refractory substance use disorders; sleep disorders; pain, such as social pain, acute pain, cancer pain, chronic pain, breakthrough pain, bone pain, soft tissue pain, nerve pain, referred pain, phantom pain, neuropathic pain, cluster headaches and migraine; obesity and eating disorders; epilepsies and seizure disorders; neuronal cell death; excitotoxic cell death; or a combination thereof.

In some embodiments, the disease, disorder or condition that is treated by activation of a serotonin receptor is psychosis or psychotic symptoms. Accordingly, the present application also includes a method of treating psychosis or psychotic symptoms comprising administering to a subject in need thereof a compound of formula (I) or a composition as described herein. The present application also includes a use of a compound of formula (I) of the present disclosure for treatment of psychosis or psychotic symptoms, as well as a use of a compound of formula (I) of the present disclosure for the preparation of a medicament for treatment of psychosis or psychotic symptoms. The application further includes a compound of formula (I) of the present disclosure for use in treating psychosis or psychotic symptoms.

In some embodiments, the disease, disorder or condition that is treated by activation of a serotonin receptor is psychosis or psychotic symptoms and the the compound of formula (I) of the present disclosure is administered in combination with one or more additional agents for psychosis or psychotic symptoms. The one or more additional agents for psychosis or psychotic symptoms may be any suitable agents known in the art, including those described herein. In some embodiments, the additional agents for psychosis or psychotic symptoms are selected typical antipsychotics and atypical antipsychotics. The typical antipsychotics may be selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, loxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine and zuclopenthixol and combinations thereof. The atypical antipsychotics may be selected from amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone and zotepine, and combinations thereof.

In some embodiments, administering to said subject in need thereof a therapeutically effective amount of the compound of formula (I) of the present disclosure does not result in a worsening of psychosis or psychotic symptoms such as, but not limited to, hallucinations and delusions. In some embodiments, administering to said subject in need thereof a therapeutically effective amount of the compound of formula (I) results in an improvement of psychosis or psychotic symptoms such as, but not limited to, hallucinations and delusions. In some embodiments, administering to said subject in need thereof a therapeutically effective amount of the compounds of formula (I) results in an improvement of psychosis or psychotic symptoms.

In some embodiments, the disease, disorder or condition that is treated by activation of a serotonin receptor is a central nervous system (CNS) disease, disorder or condition and/or a neurological disease, disorder or condition. Accordingly, the present application also includes a method of treating a CNS disease, disorder or condition and/or a neurological disease, disorder or condition comprising administering a therapeutically effective amount of compound of formula (I) or a composition of the present disclosure to a subject in need thereof. The present application also includes a use of compound of formula (I) of the present disclosure for treatment a CNS disease, disorder or condition and/or a neurological disease, disorder or condition, as well as a use of compound of formula (I) of the present disclosure for the preparation of a medicament for treatment of a CNS disease, disorder or condition and/or a neurological disease, disorder or condition. The application further includes a compound of formula (I) of the present disclosure of the application for use in treating a CNS disease, disorder or condition and/or a neurological disease, disorder or condition.

In some embodiments, the disease, disorder or condition that is treated by activation of a serotonin receptor is a central nervous system (CNS) disease, disorder or condition and/or a neurological disease, disorder or condition and the compound of formula (I) of the present disclosure is administered in combination with one or more additional agents for a central nervous system (CNS) disease, disorder or condition and/or a neurological disease, disorder or condition. The one or more additional agents for a central nervous system (CNS) disease, disorder or condition and/or a neurological disease, disorder or condition may be any suitable agents known in the art, including those described herein. In some embodiments, the additional agents for a central nervous system (CNS) disease, disorder or condition and/or a neurological disease, disorder or condition are selected from lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Non limiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline.

In some embodiments, the disease, disorder or condition that is treated by activation of a serotonin receptor is selected from attention deficit hyperactivity disorder and attention deficit disorder and a combination thereof. Accordingly, the present application also includes a method of treating attention deficit hyperactivity disorder and/or attention deficit disorder comprising administering to a subject in need thereof a compound of formula (I) or a composition as described herein. The present application also includes a use of a compound of formula (I) of the present disclosure for treatment of attention deficit hyperactivity disorder and/or attention deficit disorder, as well as a use of a compound of formula (I) of the present disclosure for the preparation of a medicament for treatment of attention deficit hyperactivity disorder and/or attention deficit disorder. The application further includes a compound of formula (I) of the present disclosure for use in treating attention deficit hyperactivity disorder and/or attention deficit disorder.

In some embodiments, the disease, disorder or condition that is treated by activation of a serotonin receptor is attention deficit hyperactivity disorder and/or attention deficit disorder and a combination thereof and the compound of formula (I) of the present disclosure is administered in combination with one or more additional agents for attention deficit hyperactivity disorder and/or attention deficit disorder and a combination thereof. The one or more additional agents for attention deficit hyperactivity disorder and/or attention deficit disorder may be any suitable agents known in the art, including those described herein. In some embodiments, the additional agents for attention deficit hyperactivity disorder and/or attention deficit disorder and a combination thereof are selected from methylphenidate, dexamphetamine, lisdexamfetine, atomoxetine and amphetamine and a combination thereof.

In some embodiments, the disease, disorder or condition that is treated by activation of a serotonin receptor is selected from dementia and Alzheimer's disease and a combination thereof. Accordingly, the present application also includes a method of treating dementia and/or Alzheimer's disease comprising administering to a subject in need thereof a compound of formula (I) or a composition as described herein. The present application also includes a use of a compound of formula (I) of the present disclosure for treatment of dementia and/or Alzheimer's disease, as well as a use of a compound of formula (I) of the present disclosure for the preparation of a medicament for treatment of dementia and/or Alzheimer's disease. The application further includes a compound of formula (I) of the present disclosure for use in treating dementia and/or Alzheimer's disease.

In some embodiments, the disease, disorder or condition that is treated by activation of a serotonin receptor is dementia or Alzheimer's disease and the compound of formula (I) of the present disclosure is administered in combination with one or more additional agents for dementia or Alzheimer's disease. The one or more additional agents for dementia or Alzheimer's disease may be any suitable agents known in the art, including those described herein. In some embodiments, the additional agents for dementia and Alzheimer's disease are selected from acetylcholinesterase inhibitors, NMDA antagonists and nicotinic agonists. The acetylcholinesterase inhibitors may be selected from donepezil, galantamine, rivastigmine, and phenserine, and combinations thereof. The NMDA antagonists may be selected from MK-801, ketamine, phencyclidine, and memantine, and combinations thereof. The nicotinic agonists may be selected from nicotine, nicotinic acid, nicotinic alpha7 agonists, or alpha2 beta4 agonists or a combination thereof.

In another aspect, the present disclosure provides a method of treating a mental illness, the method comprising administering to a subject in need thereof a compound of formula (I) or a pharmaceutical composition as described herein. In another aspect, the present disclosure provides a method of preventing a mental illness, the method comprising administering to a subject in need thereof a compound of formula (I) or a pharmaceutical composition as described herein. The mental illness may be a neuropsychiatric condition.

In certain embodiments, the mental illness is selected from anxiety disorders such as generalized anxiety disorder, panic disorder, social anxiety disorder and specific phobias; depression such as, hopelessness, loss of pleasure, fatigue and suicidal thoughts; mood disorders, such as depression, bipolar disorder, cancer-related depression, anxiety and cyclothymic disorder; psychotic disorders, such as hallucinations, delusions, mania, schizophrenia, schizoaffective disorder, schizophreniform Disorder; impulse control and addiction disorders, such as pyromania (starting fires), kleptomania (stealing) and compulsive gambling; alcohol addiction; drug addiction, such as opioid addiction/dependence, nicotine dependence, cocaine dependence, marijuana abuse and so on; smoking cessation; personality disorders, such as antisocial personality disorder, aggression, obsessive-compulsive personality disorder and paranoid personality disorder; obsessive-compulsive disorder (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” and depersonalization disorder; factitious disorders; sexual and gender disorders, such as sexual dysfunction, gender identity disorder and the paraphilias; somatic symptom disorders, formerly known as a psychosomatic disorder or somatoform disorder.

In certain embodiments, the mental illness is selected from hallucinations and delusions and a combination thereof. In these embodiments, the hallucinations may be selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations and chronoceptive hallucinations, and a combination thereof.

In another aspect, the present disclosure provides a method for treating a central nervous system (CNS) disease, disorder or condition and/or a neurological disease, disorder or condition, the method comprising administering to a subject in need thereof a compound of formula (I) or a pharmaceutical composition as described herein.

In another aspect, the present disclosure provides a method for preventing a central nervous system (CNS) disease, disorder or condition and/or a neurological disease, disorder or condition, the method comprising administering to a subject in need thereof a compound of formula (I) or a pharmaceutical composition as described herein.

In some embodiments, the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; Tic disorder; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

In another aspect, the present disclosure provides a method for increasing neuronal plasticity, the method comprising contacting a neuronal cell with a compound of formula (I) or a pharmaceutical composition as described herein, in an amount sufficient to increase neuronal plasticity of the neuronal cell. “Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In another aspect the present disclosure provides methods of treating weight, comprising administering an effective amount of a compound of the invention to a subject in need thereof. Treatment of weight may include treating weight gain; weight loss; metabolic disorder; weight gain associated with pharmaceutical intervention; weight gain associated with a mental illness (including those described herein); eating disorders such as anorexia, bulimia, cachexia, etc.; eating behaviour; obesity; diabetes; insulin resistance; pre-diabetes; glucose intolerance; hyperlipidemia; and cardiovascular disease.

In another aspect, the present disclosure provides a method for increasing dendritic spine density, the method comprising contacting a neuronal cell with a compound of formula (I) or a pharmaceutical composition as described herein, in an amount sufficient to increase dendritic spine density of the neuronal cell.

In certain embodiments, the compound of formula (I) produces a maximum number of dendritic crossings with an increase of greater than 1.0 fold by a Sholl Analysis.

In another aspect the present disclosure provides a method for activating a serotonin receptor in a cell, either in a biological sample or in a patient, comprising administering a compound of formula (I) as defined in any one of the herein disclosed embodiments to the cell. The serotonin receptor may be a 5-HT receptor subtype, preferably one or both of 5-HT_{2A} and 5-HT_{2C}.

In some embodiments, effective amounts vary according to factors such as the disease state, age, sex and/or weight of the subject or species. In some embodiments, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated and the like, but can nevertheless be routinely determined by one skilled in the art.

In some embodiments, the compounds of formula (I) of the present disclosure are administered one, two, three or four times a year. In some embodiments, the compounds of the present disclosure are administered at least once a week. However, in another embodiment, the compounds are administered to the subject from about one time per two weeks, three weeks or one month. In another embodiment, the compounds are administered about one time per week to about once daily. In another embodiment, the compounds are administered 1, 2, 3, 4, 5 or 6 times daily. The length of the treatment period depends on a variety of factors, such as the severity of the disease, disorder or condition, the age of the subject, the concentration and/or the activity of the compounds of the application and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In some embodiments, the compounds of the application are administered at doses that are hallucinogenic or psychotomimetic and taken in conjunction with psychotherapy or therapy and may occur once, twice, three, or four times a year. However, in some embodiments, the compounds are administered to the subject once daily, once every two days, once every 3 days, once a week, once every two weeks, once a month, once every two months, or once every three months at doses that are not hallucinogenic or psychotomimetic.

A compound of formula (I) of the present disclosure may be either used alone or in combination with other known agents useful for treating diseases, disorders or conditions by activation of a serotonin receptor, such as the compounds of the present disclosure. When used in combination with other known agents useful in treating diseases, disorders by activation of a serotonin receptor, it is an embodiment that a compound of formula (I) is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of formula (I) of the present disclosure is administered with another therapeutic agent simultaneously or sequentially in separate unit dosage forms or together in a single unit dosage form. Accordingly, the present application provides a single unit dosage form comprising one or more compounds of formula (I) as described herein, an additional therapeutic agent and a pharmaceutically acceptable carrier.

In some embodiments, the compounds of the application are used or administered in an effective amount which comprises administration of doses or dosage regimens that are devoid of clinically meaningful psychedelic/psychotomimetic actions. In some embodiments, the compounds of the application are used or administered in an effective amount which comprises administration of doses or dosage regimens that provide clinical effects similar to those exhibited by a human plasma psilocin Cmax of 4 ng/mL or less and/or human 5-HT_{2A} human CNS receptor occupancy of 40% or less or those exhibited by a human plasma psilocin Cmax of 1 ng/mL or less and/or human 5-HT_{2A} human CNS receptor occupancy of 30% or less. In some embodiments, the compounds of the application are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

### Kit

In another embodiment there is provided a kit or article of manufacture including one or more compounds, pharmaceutically acceptable salt, stereoisomer, solvate, metabolite, or polymorph, and/or pharmaceutical compositions as described above.

In other embodiments there is provided a kit for use in a therapeutic application mentioned above, the kit including:
a container holding one or more compounds, pharmaceutically acceptable salt, stereoisomer, solvate, metabolite, or polymorph and/or pharmaceutical compositions as described herein;
a label or package insert with instructions for use.

It will be understood that the invention disclosed and defined in this specification extends to all alternative combinations of two or more of the individual features mentioned or evident from the text or drawings. All of these different combinations constitute various alternative aspects of the invention.

### EXAMPLES

Scheme 1: Compounds of general formula (I) can be synthesised from the appropriately substituted aniline following the outlined sequence of steps in Scheme 1 or similar as one skilled in the art may consider. Hydrazine formation provided a suitable intermediate to undergo Fischer indole synthesis to generate substituted indole intermediates. Dehalogenation provided access to compounds of general formula (I) (exemplified by P-4). One skilled in the art will recognise that utilising differentially substituted amines during the Fischer indole synthesis would allow access to compounds of general formula (I) disclosed herein.

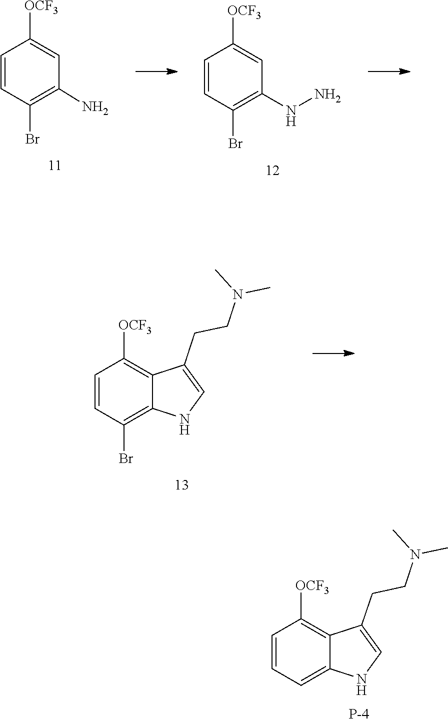

### Example 1: N,N-dimethyl-2-(4-(trifluoromethoxy)-1H-indol-3-yl)ethan-1-amine (P-4)

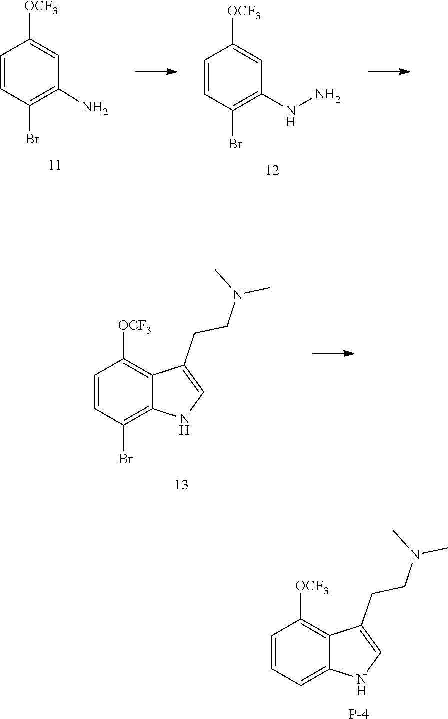

### Step 1: (2-bromo-5-(trifluoromethoxy)phenyl)hydrazine (12)

To a cooled (0° C.) solution of 2-bromo-5-(trifluoromethoxy)aniline (9.0 g, 35.2 mmol) in conc. HCl (90 mL) and water (31.5 mL) was added a solution of NaNO₂ (2.7 g, 38.7 mmol) in H₂O (31.5 mL) over a period of 20 min. The reaction mixture was stirred at 0° C. for 1 h, followed by dropwise addition of a solution of SnCl₂ (23.8 g, 106 mmol, 3.0 eq) in conc. HCl (90 mL) at 0° C. during a period of 1 h. After 2-bromo-5-(trifluoromethoxy)aniline was consumed as indicated by TLC analysis, the reaction was basified with 50% aqueous NaOH (˜250 mL) at 0° C. until the pH˜14. The mixture was extracted with CH₂Cl₂ (200 mL×3), and the combined organic layer was dried over anhydrous Na₂SO₄, filtered, and concentrated under reduced pressure. The crude material was purified by flash chromatography (EtOAc/Hexane, 1:10) to provide (2-bromo-5-(trifluoromethoxy)phenyl)hydrazine (7.4 g, 78%) as a yellow oil. ¹H NMR (300 MHz, CDCl₃): δ 7.38 (d, J=8.7 Hz, 1H), 7.04 (s, 1H), 6.52 (d, J=8.4 Hz, 1H), 5.80 (br s, 1H), 3.62 (br s, 2H).

### Step 2: 2-(7-bromo-4-(trifluoromethoxy)-1H-indol-3-yl)-N,N-dimethylethan-1-amine (13)

A solution of (2-bromo-5-(trifluoromethoxy)phenyl)hydrazine (6.0 g, 22.1 mmol, 1.0 eq) and 4,4-dimethoxy-N,N-dimethylbutan-1-amine (3.9 g, 24.4 mmol, 1.1 eq) in toluene (90 mL) was stirred at 60° C. for 30 min. Phosphoric acid (90 mL, 85%) was added to the mixture at 60° C. and stirred for 1 h. The mixture was cooled to ambient temperature and poured into ice-water (450 mL). The mixture was basified with 50% (w/v) aq. NaOH solution (130 mL) to pH˜13, followed by extraction with EtOAc (300 mL×2). The combined organic layer was dried over anhydrous Na₂SO₄, filtered, and evaporated under reduced pressure. The crude product was purified by column chromatography (SiO₂, CH₂Cl₂/MeOH, 100:1 to 30:1) to afford 2-(7-bromo-4-(trifluoromethoxy)-1H-indol-3-yl)-N,N-dimethylethan-1-amine (680 mg, 8.7%) as an off-white solid. ¹H NMR (300 MHz, DMSO-d₆): δ 11.6 (brs, 1H), 7.46-7.36 (m, 2H), 6.96-6.93 (m, 1H), 3.18-2.95 (m, 4H), 2.46 (s, 6H).

### Step 3: N,N-dimethyl-2-(4-(trifluoromethoxy)-1H-indol-3-yl)ethan-1-amine (P-4)

A stirred mixture of 2-(7-bromo-4-(trifluoromethoxy)-1H-indol-3-yl)-N,N-dimethylethan-1-amine (680 mg, 1.51 mmol, 1.0 eq), K₂CO₃ (418 mg, 3.03 mmol, 2.0 eq) and 10% Pd/C (80 mg) in MeOH (15 mL) was hydrogenated under 0.5 MPa of H₂ at 40° C. for 3 h and then filtered through a pad of Celite. The filter cake was washed with EtOAc (10 mL×3), and the combined filtrate was concentrated in vacuo. The residue was dissolved in EtOAc (500 mL) and washed with H₂O (200 mL×2). The organic layer was dried over anhydrous Na₂SO₄, filtered, and the solvent evaporated to give a yellow oil. Purification by preparative HPLC provided N,N-dimethyl-2-(4-(trifluoromethoxy)-1H-indol-3-yl)ethan-1-amine as a solid (170 mg, 32%). ¹H NMR (300 MHz, DMSO-d₆): δ 11.61 (br s, 1H), 7.36-7.42 (m, 2H), 6.94 (d, J=8.3 Hz, 1H), 2.87-3.07 (m, 4H), 2.52 (s, 6H). LCMS (ESI+): m/z 273.0 [M+H]⁺ HPLC Purity (220 nm): 97.8%

Scheme 2: Compounds of general formula (I) can be synthesised from the appropriately substituted hydrazine following the outlined sequence of steps in Scheme 2 or similar as one skilled in the art may consider. Fischer indole synthesis allows access to the appropriately substituted indoles providing access to compounds of general formula (I) (exemplified by P-5). One skilled in the art will recognise that utilising differentially substituted amines would allow access to compounds of general formula (I) disclosed herein.

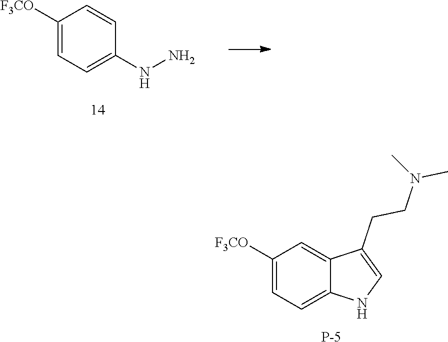

### Example 7: N,N-dimethyl-2-(5-(trifluoromethoxy)-1H-indol-3-yl)ethan-1-amine (P-5)

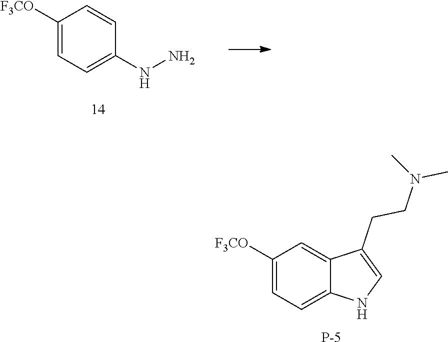

Step 1: N,N-dimethyl-2-(5-(trifluoromethoxy)-1H-indol-3-yl)ethan-1-amine (P-5) To a solution of (4-(trifluoromethoxy)phenyl)hydrazine hydrochloride (458 mg, 2.0 mmol) in 4 wt. % aqueous H₂SO₄ (12 mL) at ambient temperature was added 4,4-dimethoxy-N,N-dimethylbutan-1-amine (387 mg, 2.4 mmol) in one portion. The reaction was stirred under reflux for 4 h. The cooled reaction mixture was basified to pH 8 with NH₄OH (12 g, 87 mmol) and extracted with EtOAc (25 mL×3). The combined organic phase was dried over anhydrous Na₂SO₄, filtered, and concentrated in vacuo. The residue was purified by preparative HPLC to give N,N-dimethyl-2-(5-(trifluoromethoxy)-1H-indol-3-yl)ethan-1-amine (186 mg) as an oil that rapidly formed a carbonate salt. The solid was dissolved in methanol (2 mL) and treated with a solution of 2 M HCl in Et₂O to afford its HCl salt. After concentration in vacuo and trituration with Et₂O, N,N-dimethyl-2-(5-(trifluoromethoxy)-1H-indol-3-yl)ethan-1-amine hydrochloride (140 mg) was obtained as a pale brown solid. ¹H NMR (300 MHz, DMSO-d₆): δ11.31 (s, 1H), 10.24-10.51 (br, 1H), 7.64 (s, 1H), 7.39-7.47 (m, 2H), 7.07 (d, J=8.7 Hz, 1H), 3.27-3.33 (m, 2H), 3.10-3.15 (m, 2H), 2.82 (s, 6H). LCMS (ESI+): m/z 273.0 [M+H]⁺. HPLC Purity (220 nm): 99.3%.

Scheme 3: Compounds of general formula (I) can be synthesised from the appropriately substituted tryptamine following the outlined sequence of steps in Scheme 3 or similar as one skilled in the art may consider. Boc protection of the pendant amine in the tryptamine scaffold followed by reduction allows access to methyltryptamine intermediates that can undergo subsequent reductive alkylation with various aldehydes and ketones to produce compounds of general formula (I) (exemplified by P-34). One skilled in the art will recognise that utilising alternate aldehydes and ketones during the reductive alkylation transformation would generate alternate compounds of general formula (I) disclosed herein.

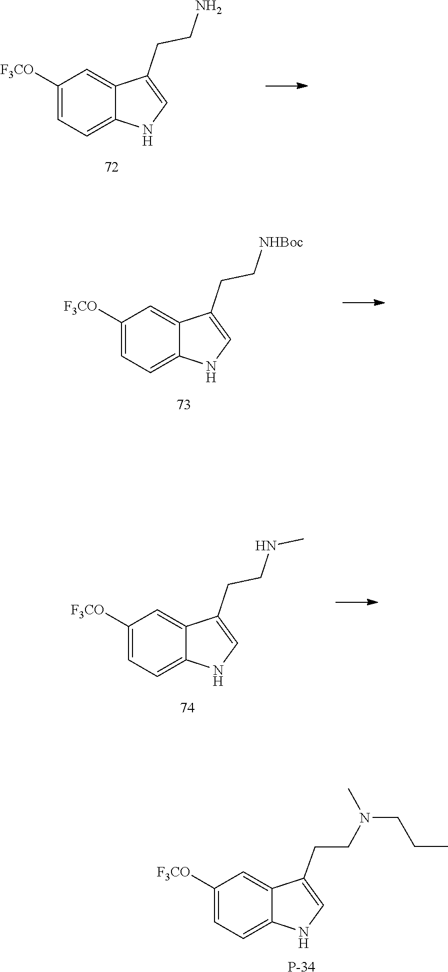

### Example 10: N-methyl-N-(2-(5-(trifluoromethoxy)-1H-indol-3-yl)ethyl)propan-1-amine (P-34)

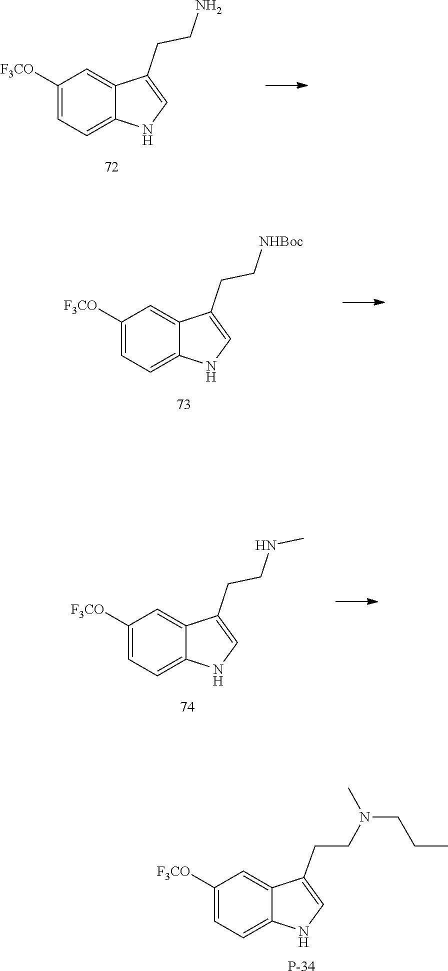

Step 1: tert-butyl (2-(5-(trifluoromethoxy)-1H-indol-3-yl)ethyl)carbamate (73) To an ice-cold (0° C.) solution of 5-(trifluoromethoxy)tryptamine hydrochloride (1.40 g, 5 mmol, 1.0 equiv.) and triethylamine (2.1 mL, 15 mmol, 3.0 equiv.) in THF (45 mL) was added a solution of Boc₂O (1.31 g, 6 mmol, 1.2 equiv.) in THF (5 mL), and the reaction stirred for 2 h. The reaction was poured onto ice-water (50 mL) and extracted with EtOAc (50 mL×3). The combined organic layers were washed with brine (50 mL), dried (MgSO₄), filtered, and the filtrate concentrated under reduced pressure. The crude product was purified by column chromatography (SiO₂, hexane/EtOAc, v/v, 1/1) to give tert-butyl (2-(5-(trifluoromethoxy)-1H-indol-3-yl)ethyl)carbamate as a pale brown solid (1.72 g, 100%). ¹H NMR (400 MHz, DMSO-d₆): δ 11.10 (s, 1H), 7.46 (s, 1H), 7.41 (d, J=8.8 Hz, 1H), 7.31-7.26 (m, 1H), 7.02 (dd, J=8.8, 1.2 Hz, 1H), 6.88 (t, J=5.8 Hz, 1H), 3.22-3.12 (m, 2H), 2.78 (t, J=7.2 Hz, 2H), 1.37 (s, 9H).

### Step 2: N-methyl-2-(5-(trifluoromethoxy)-1H-indol-3-yl)ethan-1-amine (74)

To an ice-cold (0° C.) solution of tert-butyl (2-(5-(trifluoromethoxy)-1H-indol-3-yl)ethyl)carbamate (1.5 g, 4.36 mmol) in THF (50 mL) under N₂ was added LiAlH₄ (496 mg, 13.1 mmol), and the mixture heated to reflux for 1 h. The mixture was then cooled to 0° C., quenched by sequential dropwise addition of cold water (0.5 mL), 3.75 M aq. NaOH (0.5 mL), and water (1.5 mL). The suspension was then stirred for 15 min with ˜1 g of anhydrous Na₂SO₄ before being filtered through a pad of Celite. The Celite plug was washed with hot THF (20 mL×2), and the combined filtrate was concentrated to afford N-methyl-2-(5-(trifluoromethoxy)-1H-indol-3-yl)ethan-1-amine as an off-white solid (1.05 g, 93%). ¹H NMR (400 MHz, DMSO-d₆): δ 11.07 (s, 1H), 7.46 (s, 1H), 7.40 (d, J=8.8 Hz, 1H), 7.28 (d, J=2.0 Hz, 1H), 7.02 (d, J=8.8 Hz, 1H), 2.83-2.75 (m, 2H), 2.75-2.67 (m, 2H), 2.30 (s, 3H). ¹³C NMR (101 MHz, DMSO-d₆): δ 141.3, 134.6, 127.4, 125.1, 114.4, 113.5, 112.3, 110.7, 52.3, 36.1, 25.0 (OCF₃ carbon peak unresolved).

### Step 3: N-methyl-N-(2-(5-(trifluoromethoxy)-1H-indol-3-yl)ethyl)propan-1-amine (P-34)

To a solution of N-methyl-2-(5-(trifluoromethoxy)-1H-indol-3-yl)ethan-1-amine (0.2 g, 0.77 mmol) and propanal (66.6 μL, 1.2 eq., 0.93 mmol) in 1,2-dichloroethane (5 mL) was added NaBH(OAc)₃ (246 mg, 1.5 eq., 1.16 mmol) at ambient temperature. The mixture was stirred for 16 h, then quenched with 1 M aq. NaOH (5 mL) and extracted with CH₂Cl₂ (10 mL×3). The combined organic layers were washed with brine (20 mL), dried over Na₂SO₄, filtered, and then concentrated under reduced pressure. The residue was purified by flash chromatography (SiO₂, 0.1% to 6% MeOH/NH₃(aq.) in CH₂Cl₂) to afford N-ethyl-N-methyl-2-(5-(trifluoromethoxy)-1H-indol-3-yl)ethan-1-amine (114 mg) as a yellow oil which was used in the next step without further purification.

### Step 3a: N-methyl-N-(2-(5-(trifluoromethoxy)-1H-indol-3-yl)ethyl)propan-1-amine fumarate (P-34-fumarate)

To a solution of fumaric acid (44 mg, 0.38 mmol) in minimal refluxing acetone was added a solution of N-methyl-N-(2-(5-(trifluoromethoxy)-1H-indol-3-yl)ethyl)propan-1-amine (114 mg) in minimal warm acetone. The resulting solution was allowed to cool to ambient temperature and stood overnight at 4° C. to afford N-methyl-N-(2-(5-(trifluoromethoxy)-1H-indol-3-yl)ethyl)propan-1-amine as the fumarate salt (115 mg, 41% over 2 steps) which were off-white crystals. ¹H NMR (400 MHz, DMSO-d₆): δ 11.15 (s, 1H), 7.55-7.46 (m, 1H), 7.41 (d, J=8.8 Hz, 1H), 7.32 (d, J=2.4 Hz, 1H), 7.03 (dd, J=8.8, 1.2 Hz, 1H), 6.52 (s, 1H), 2.95-2.85 (m, 2H), 2.84-2.74 (m, 2H), 2.59-2.52 (m, 2H), 2.41 (s, 3H), 1.51 (sext, J=7.4 Hz, 2H), 0.86 (t, J=7.4 Hz, 3H). ¹³C NMR (101 MHz, DMSO-d₆): δ 167.4, 141.4, 134.8, 134.6, 127.2, 125.2, 120.5 (d, J=253.8 Hz), 114.4, 112.5, 112.3, 110.6, 58.1, 56.9, 40.8, 21.5, 18.9, 11.5. qNMR Purity (ERETIC): 100%.

Scheme 4: Compounds of general formula (I) can be synthesised from the appropriately substituted indole in a single step as outlined in Scheme 4 or similar as one skilled in the art may consider. Substituted tryptamine cores could undergo reductive alkylation to access compounds of general formula (I) (exemplified by P-15). One skilled in the art will recognise that protecting the amine with a suitable protecting group such as benzyl, followed by alkylation, subsequent deprotection and a second alkylation would allow access to differentially alkylated compounds of general formula (I) disclosed herein.

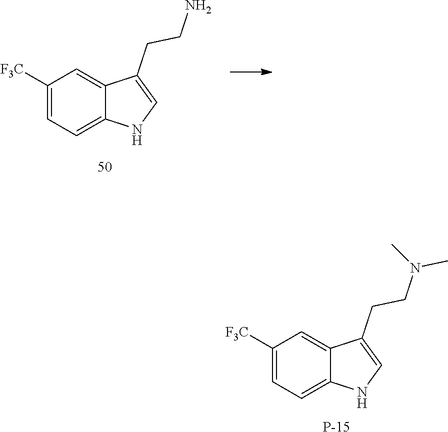

### Example 14: N,N-dimethyl-2-(5-(trifluoromethyl)-1H-indol-3-yl)ethan-1-amine (P-15)

### Step 1: N,N-dimethyl-2-(5-(trifluoromethyl)-1H-indol-3-yl)ethan-1-amine (P-15)

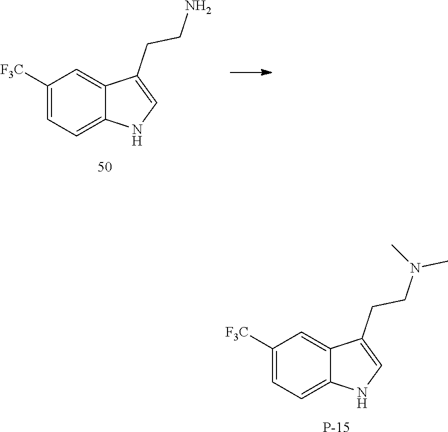

A solution of 2-(5-(trifluoromethyl)-1H-indol-3-yl)ethan-1-amine (0.2 g, 876 μmol) in MeOH (5 mL) was treated with AcOH (52.6 mg, 876 μmol), 37% aqueous formaldehyde solution (356 mg, 5 eq., 4.38 mmol), and NaBH₃CN (220 mg, 4 eq., 3.51 mmol), and stirred at ambient temperature for 12 h. The reaction was diluted with H₂O:EtOAc (1:1-100 mL), the layers separated, and the organic layer was dried over anhydrous Na₂SO₄, filtered, and concentrated in vacuo. The crude product was purified by preparative HPLC to afford N,N-dimethyl-2-(5-(trifluoromethyl)-1H-indol-3-yl)ethan-1-amine (54 mg, 24%) as a pale yellow solid. HPLC purity: 96.6% (220 nm); LCMS (ESI+) m/z 257.1 [M+H]⁺; ¹H NMR (400 MHz, CDCl₃): δ 8.23 (br. s, 1H), 7.90 (s, 1H), 7.42 (s, 2H), 7.18-7.12 (m, 1H), 3.01-2.93 (m, 2H), 2.70-2.62 (m, 2H), 2.36 (s, 6H).

Scheme 13: Compounds of general formula (I) can be synthesised from the appropriately substituted indole following the outlined sequence of steps in Scheme 13 or similar as one skilled in the art may consider. An appropriately substituted indole could be glyoxylated with oxalyl chloride followed by treatment with an appropriately substituted amine to give glyoxamide intermediates. Such intermediates could then be subjected to reductive conditions to provide access to compounds of general formula (I) (exemplified by P-16, P-17, and P-18).

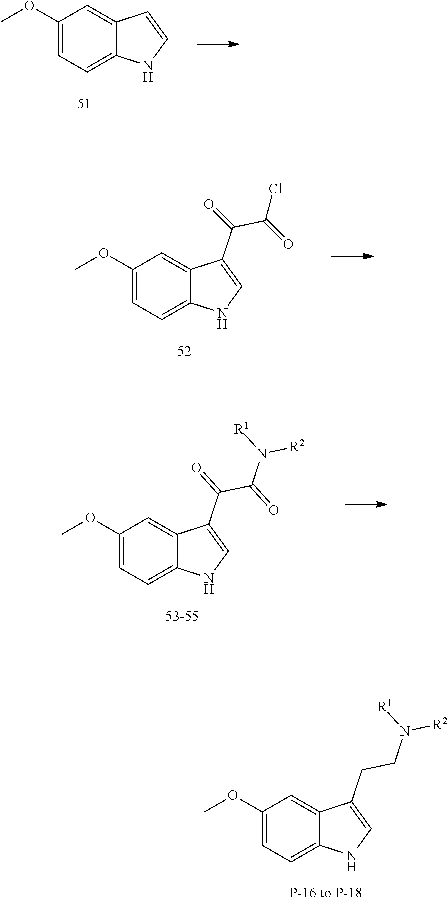

### Example 19: N-(2-(5-methoxy-1H-indol-3-yl)ethyl)-N-methylbutan-2-amine (P-16)

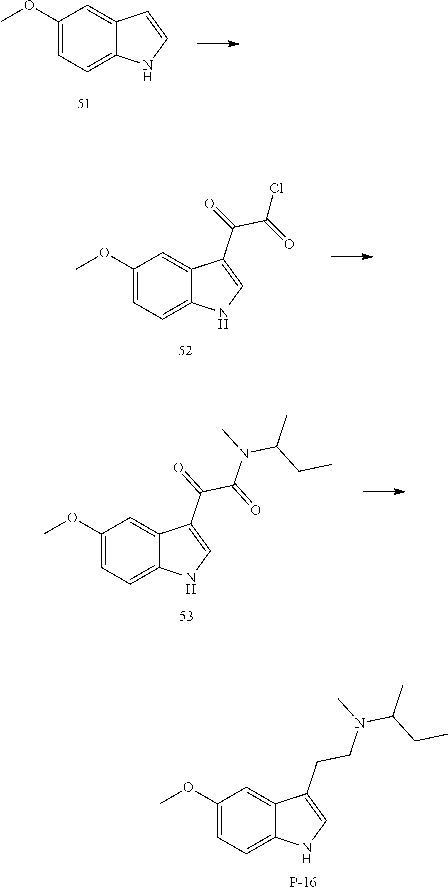

### Step 1: 2-(5-methoxy-1H-indol-3-yl)-2-oxoacetyl chloride (52)

To a solution of (COCl)₂ (2 g, 15.7 mmol) in Et₂O (36 mL) at 0° C. was added dropwise a solution of 5-methoxy-1H-indole (2 g, 13.6 mmol) in minimal Et₂O. The reaction was stirred at 0° C. for 30 min and the resultant precipitate collected by filtration. The crude solid was dried under vacuum to provide 2-(5-methoxy-1H-indol-3-yl)-2-oxoacetyl chloride (2.9 g, 78%). ¹H NMR (300 MHz, CDCl₃): δ 8.88 (s, 1H), 8.26 (d, J=3.2 Hz, 1H), 7.89 (s, 1H), 7.36 (d, J=8.9 Hz, 1H), 7.00 (d, J=7.6 Hz, 1H), 3.91 (s, 3H).

### Step 2: N-(sec-butyl)-2-(5-methoxy-1H-indol-3-yl)-N-methyl-2-oxoacetamide (53)

To a solution of 2-(5-methoxy-1H-indol-3-yl)-2-oxoacetyl chloride (300 mg, 1.26 mmol) in CH₂Cl₂ (10 mL) was added N-methylbutan-2-amine (331 mg, 3.79 mmol). The reaction mixture was stirred at ambient temperature for 2 h. The reaction mixture was quenched with H₂O (20 mL). The layers were separated, and the aqueous phase was extracted with CH₂Cl₂ (2×20 mL). The combined organics were dried over anhydrous Na₂SO₄, filtered, and the filtrate concentrated in vacuo to provide N-(sec-butyl)-2-(5-methoxy-1H-indol-3-yl)-N-methyl-2-oxoacetamide (300 mg, 81%) as a yellow solid. ¹H NMR (300 MHz, DMSO-d₆): δ 12.17 (s, 1H), 7.96 (m, 1H), 7.60 (s, 1H), 7.43 (dd, J=8.9, 5.9 Hz, 1H), 6.90 (dt, J=8.8, 2.7 Hz, 1H), 3.80 (s, 3H), 3.34 (s, 3H), 3.12-2.96 (m, 1H), 1.81-1.32 (m, 2H), 1.16 (d, J=6.6 Hz, 3H), 0.90 (t, J=7.5 Hz, 3H).

### Step 3: N-(2-(5-methoxy-1H-indol-3-yl)ethyl)-N-methylbutan-2-amine (P-16)

To a solution of LiAlH₄ (395 mg, 10.4 mmol) in THF (10 mL) under reflux was added N-(sec-butyl)-2-(5-methoxy-1H-indol-3-yl)-N-methyl-2-oxoacetamide (300 mg, 1.04 mmol) in THF (10 mL). The reaction mixture was stirred under reflux for 1 h. The resulting mixture was quenched with H₂O (0.4 mL), 15% NaOH aqueous solution (0.4 mL) and H₂O (1.2 mL), and then MgSO₄ and EtOAc were added. The mixture was stirred at ambient temperature for 30 min. The mixture was filtered through Celite pad. The filtrate was concentrated to give the crude product. The resulting residue was purified by flash column chromatography with an isocratic elution of CH₂Cl₂ (95%) and MeOH (5%) to provide N-(2-(5-methoxy-1H-indol-3-yl)ethyl)-N-methylbutan-2-amine (120 mg, 44%) as an off-white solid. ¹H NMR (300 MHz, MeOH-d₄): δ 7.28 (d, J=9.0 Hz, 1H), 7.20 (s, 1H), 7.09 (d, J=1.8 Hz, 1H), 6.80 (dd, J=2.4, 9.0 Hz, 1H), 3.84 (s, 3H), 3.60-3.33 (m, 3H), 3.20-3.18 (m, 2H), 2.86 (s, 3H), 1.88-1.74 (m, 2H), 1.31 (brs, 3H), 0.99 (brs, 3H). LCMS (ESI+): m/z 261.1 [M+H]⁺. HPLC Purity (220 nm): 97.1%

### Example-20: N-isopropyl-N-(2-(5-methoxy-1H-indol-3-yl)ethyl)propan-1-amine (P-17)

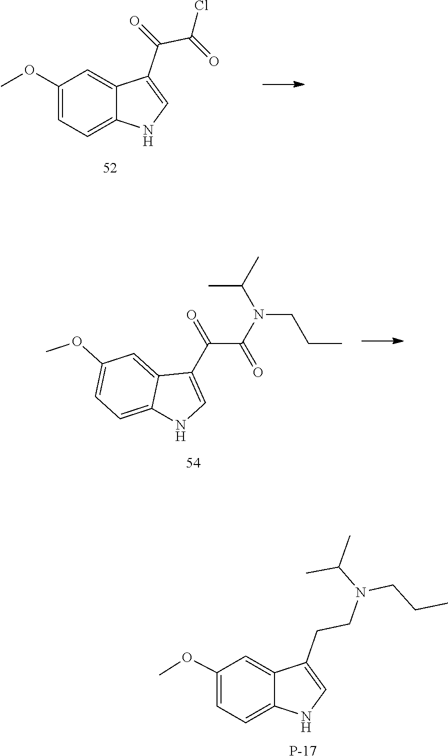

### Step 1: N-isopropyl-2-(5-methoxy-1H-indol-3-yl)-2-oxo-N-propylacetamide (54)

To a solution of 2-(5-methoxy-1H-indol-3-yl)-2-oxoacetyl chloride (300 mg, 1.27 mmol) in CH₂Cl₂ (12 mL) was added N-isopropylpropan-1-amine (639 mg, 6.33 mmol). The reaction was stirred at 0° C. for 1 h. The resulting mixture was quenched with H₂O (15 mL) and extracted with CH₂Cl₂ (3×15 mL). The combined organics were dried over anhydrous Na₂SO₄, filtered, and concentrated in vacuo to provide the crude N-isopropyl-2-(5-methoxy-1H-indol-3-yl)-2-oxo-N-propylacetamide (387 mg) as a white solid. ¹H NMR (300 MHz, CDCl₃): δ 9.06 (br s, 1H), 7.82 (s, 1H), 7.78-7.73 (m, 1H), 7.30 (d, J=9.0 Hz, 1H), 6.93 (dd, J=9.0, 2.5 Hz, 1H), 4.10-4.04 (m, 1H), 3.90 (s, 3H), 3.43-3.12 (m, 2H), 1.85-1.67 (m, 2H), 1.33 (d, J=6.9 Hz, 2H), 1.19 (d, J=6.6 Hz, 4H), 1.00 (t, J=7.4 Hz, 2H), 0.77 (t, J=7.3 Hz, 1H) [mixture of rotamers].

### Step 2: N-isopropyl-N-(2-(5-methoxy-1H-indol-3-yl)ethyl)propan-1-amine (P-17)

To a refluxing solution of LiAlH₄ (289 mg, 7.62 mmol) in THF (10 mL) was added dropwise a solution of N-isopropyl-2-(5-methoxy-1H-indol-3-yl)-2-oxo-N-propylacetamide (230 mg, 0.762 mmol) in THF (10 mL) under N₂. The reaction mixture was stirred for 30 min under reflux. After cooling to 0° C., the reaction mixture was quenched by addition of H₂O (0.29 mL), followed by 15% aqueous NaOH solution (0.29 mL) and H₂O (0.87 mL). The mixture was dried over anhydrous MgSO₄, filtered, and the filtrate concentrated in vacuo. The resulting mixture was purified by flash column chromatography with an isocratic elution (EtOAc) to provide N-isopropyl-N-(2-(5-methoxy-1H-indol-3-yl)ethyl)propan-1-amine (145 mg, 70%) as a yellow oil. ¹HNMR (300 MHz, MeOD-d₄): δ 7.27 (d, J=9.0 Hz, 1H), 7.20 (s, 1H), 7.07 (d, J=2.1 Hz, 1H), 6.82-6.78 (m, 1H), 3.84 (s, 3H), 3.82-3.77 (m, 1H), 3.44-3.33 (m, 2H), 3.23-3.10 (m, 4H), 1.82-1.76 (m, 2H), 1.38-1.34 (m, 6H), 1.02 (t, J=7.2 Hz, 3H). LCMS (ESI+): m/z 275.3 [M+H]⁺. HPLC Purity (200 nm): 97.7%

### Example 21: N-ethyl-N-(2-(5-methoxy-1H-indol-3-yl)ethyl)-2-methylpropan-1-amine (P-18)

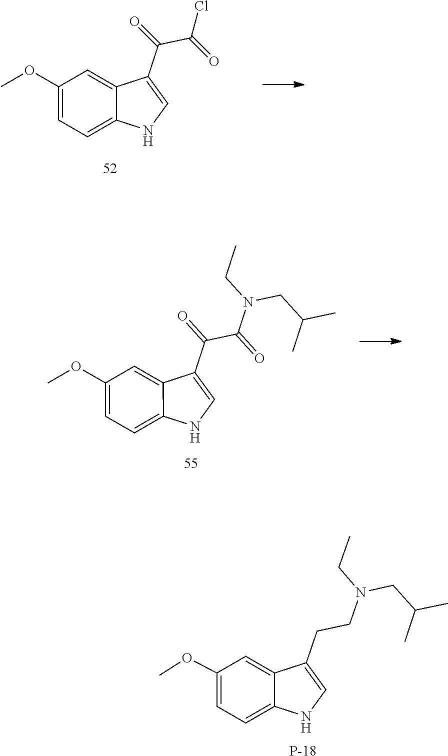

### Step 1: N-ethyl-N-isobutyl-2-(5-methoxy-1H-indol-3-yl)-2-oxoacetamide (55)

To a solution of 2-(5-methoxy-1H-indol-3-yl)-2-oxoacetyl chloride (300 mg, 1.26 mmol) in CH₂Cl₂ (10 mL) was added N-ethyl-2-methylpropan-1-amine hydrochloride (520 mg, 3.79 mmol) and triethylamine (1.92 g, 18.99 mmol) at ambient temperature. The reaction mixture was stirred at ambient temperature for 16 h. The reaction mixture was quenched with H₂O (20 mL). The layers were separated and the aqueous phase was extracted with CH₂Cl₂ (2×20 mL). The combined organics were dried over anhydrous Na₂SO₄, filtered, and concentrated in vacuo to provide N-ethyl-N-isobutyl-2-(5-methoxy-1H-indol-3-yl)-2-oxoacetamide (200 mg, 52%) as a yellow solid which was used immediately in the next step without further purification.

### Step 2: N-ethyl-N-(2-(5-methoxy-1H-indol-3-yl)ethyl)-2-methylpropan-1-amine (P-18)

To a solution of LiAlH₄ (250 mg, 6.6 mmol) in THF (10 mL) under reflux was added N-ethyl-N-isobutyl-2-(5-methoxy-1H-indol-3-yl)-2-oxoacetamide (200 mg, 0.66 mmol) in THF (10 mL). The reaction mixture was stirred under reflux for 1 h. The resulting mixture was quenched with H₂O (0.3 mL), 15% NaOH aqueous solution (0.3 mL) and H₂O (0.9 mL), and then MgSO₄ and EtOAc were added. The mixture was stirred at ambient temperature for 30 min. The mixture was filtered through Celite pad. The filtrate was concentrated to give the crude product which was purified by flash column chromatography with an isocratic elution of CH₂Cl₂:MeOH (19:1 v/v) to provide N-ethyl-N-(2-(5-methoxy-1H-indol-3-yl)ethyl)-2-methylpropan-1-amine (45 mg, 11%) as a yellow oil. ¹H NMR (300 MHz, MeOD-d₄): δ 7.26 (d, J=9.0 Hz, 1H), 7.19 (s, 1H), 7.06 (d, J=2.1 Hz, 1H), 6.81 (dd, J=9.0, 2.4 Hz, 1H), 3.84 (s, 3H), 3.48-3.36 (m, 4H), 3.22-3.16 (m, 2H), 3.11-3.04 (m, 2H), 2.18-2.10 (m, 1H) 1.36 (t, J=7.2 Hz, 3H), 1.03 (t, J=6.3 Hz, 6H). LCMS (ESI+): m/z 275.4 [M+H]⁺. HPLC Purity (220 nm): 95.9%

Scheme 14: Compounds of general formula (I) can be synthesised from the appropriately substituted tryptamine following the outlined sequence of steps in Scheme 14 or similar as one skilled in the art may consider. An appropriately substituted tryptamine can undergo sequential one-pot reductive alkylations to access to compounds of general formula (I) (exemplified by P-19).

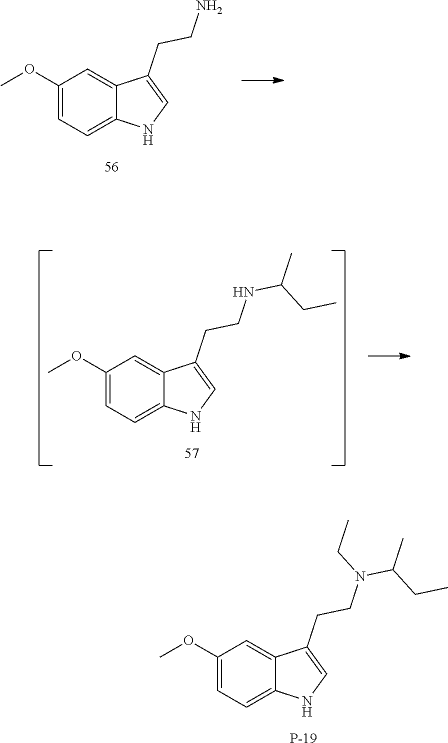

### Example 22: N-ethyl-N-(2-(5-methoxy-1H-indol-3-yl)ethyl)butan-2-amine (P-19)

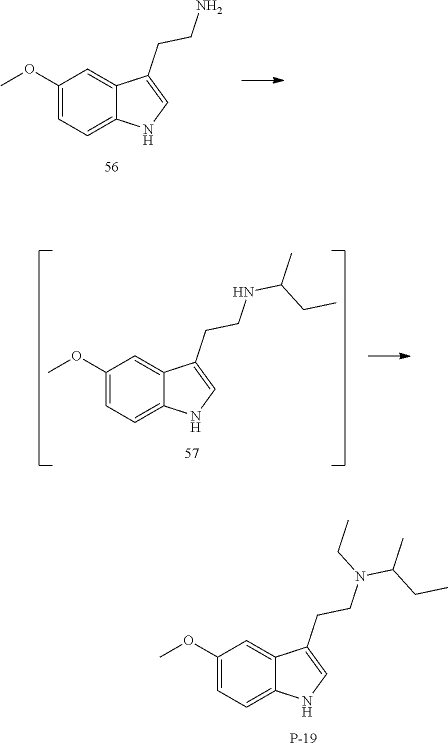

### Step 1: N-ethyl-N-(2-(5-methoxy-1H-indol-3-yl)ethyl)butan-2-amine (P-19)

To a solution of 2-(5-methoxy-1H-indol-3-yl)ethan-1-amine (500 mg, 2.63 mmol) in MeOH (10 mL) was added butan-2-one (576 mg, 7.89 mmol), NaBH₃CN (663 mg, 10.52 mmol) and AcOH (80 mg, 1.33 mmol). The reaction mixture was stirred at ambient temperature for 2 h. Then acetaldehyde (463 mg, 10.52 mmol) and NaBH₃CN (663 mg, 10.52 mmol) were added. The reaction mixture was stirred at 45° C. for 2 h. The reaction was concentrated in vacuo to give the crude product. The resulting residue was purified by flash column chromatography (SiO₂, CH₂Cl₂/MeOH, v/v, 91/9) to provide the crude product (400 mg) which was dissolved in methanol (1 mL) and treated with HCl in Et₂O (8 mL, 1 M). The reaction was stirred at ambient temperature for 10 min and then concentrated in vacuo. The resulting solid was washed with Et₂O (3×3 mL) to afford N-ethyl-N-(2-(5-methoxy-1H-indol-3-yl)ethyl)butan-2-amine as the hydrochloride salt (50.2 mg, 6%) which was a pale yellow solid. ¹H NMR (300 MHz, MeOD-d₄): δ 7.27 (d, J=9.0 Hz, 1H), 7.20 (s, 1H), 7.08-7.04 (m, 1H), 6.82-6.79 (m, 1H), 3.83 (s, 3H), 3.50-3.34 (m, 5H), 3.24-3.19 (m, 2H), 1.94-1.74 (m, 1H), 1.68-1.55 (m, 1H), 1.38 (t, J=7.2 Hz, 3H), 1.36-1.31 (m, 3H) 1.05-0.94 (m, 3H). LCMS (ESI+): m/z 275.3 [M+H]⁺. HPLC Purity (254 nm): 99.6%

Scheme 15: Compounds of general formula (I) can be synthesised from the appropriately substituted tryptamine following the outlined sequence of steps in Scheme 15 or similar as one skilled in the art may consider. An appropriately substituted tryptamine can undergo sequential one-pot reductive alkylations to access compounds of general formula (I) (exemplified by P-20 and P-21).

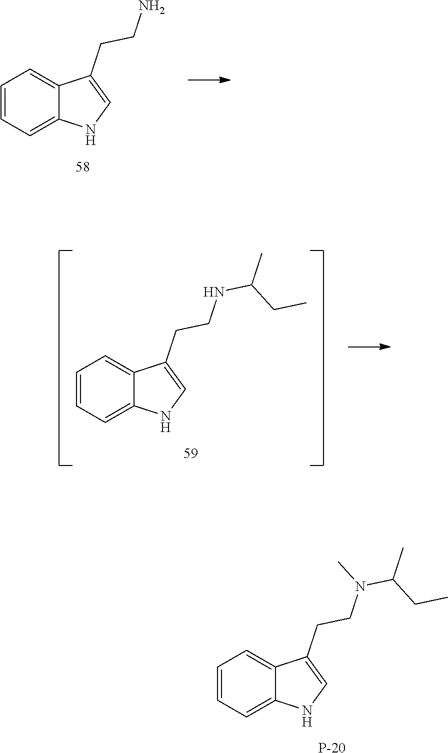

### Example 23: N-(2-(1H-indol-3-yl)ethyl)-N-methylbutan-2-amine (P-20)

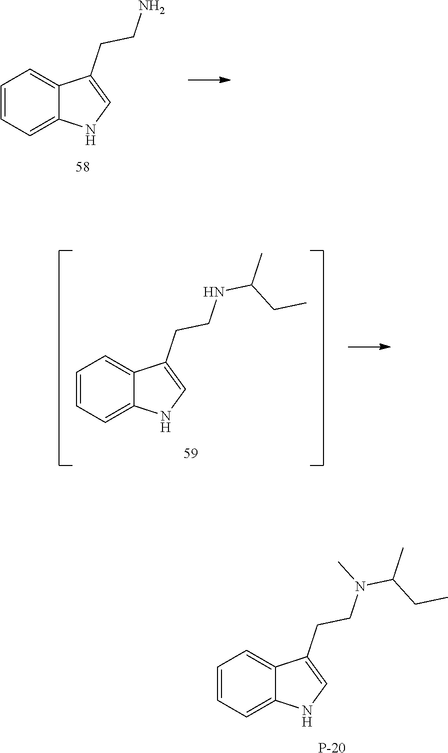

### Step 1: N-(2-(1H-indol-3-yl)ethyl)-N-methylbutan-2-amine (P-20)

To a solution of tryptamine (200 mg, 1.25 mmol) in MeOH (12 mL) was added butan-2-one (270 mg, 3.75 mmol), NaBH₃CN (315 mg, 5.00 mmol), AcOH (23 mg, 0.38 mmol). The reaction was stirred at ambient temperature for 3 h. Then an aqueous formaldehyde solution (0.37 mL, 5.00 mmol, 37-40%) and NaBH₃CN (315 mg, 5.00 mmol) were added to the reaction. The mixture was stirred at ambient temperature for 1 h, and then quenched with H₂O (30 mL) and extracted with EtOAc (30 mL×2). The combined organic layers were washed with brine (40 mL), dried over Na₂SO₄ (15 g), filtered, and concentrated. The resulting residue was purified by flash silica chromatography (CH₂Cl₂-MeOH, 30:1 to 20:1) to afford the crude product which was dissolved in CH₂Cl₂ (5 mL) and treated with HCl in Et₂O (4 mL, 1 N) dropwise over 2 minutes, and stirred at ambient temperature for 30 min. The mixture was then concentrated and the resulting solid purified by preparative HPLC to afford N-(2-(1H-indol-3-yl)ethyl)-N-methylbutan-2-amine as the hydrochloride salt (89 mg, 27%) which was an off-white solid. ¹H NMR (300 MHz, DMSO-d₆): δ 11.03 (br s, 1H), 10.05 (br s, 1H), 7.69 (m, 1H), 7.36 (d, J=7.8 Hz, 1H), 7.26 (s, 1H), 7.11-6.98 (m, 2H), 3.41-3.18 (m, 5H), 2.78-2.70 (m, 3H), 2.04-1.82 (m, 1H), 1.58-1.39 (m, 1H), 1.31-1.20 (m, 3H), 1.00-0.85 (m, 3H). LCMS (ESI+): m/z 231.2 [M+H]⁺. HPLC Purity (220 nm): 99.9%

### Example 24: N-(2-(1H-indol-3-yl)ethyl)-N-ethylbutan-2-amine (P-21)

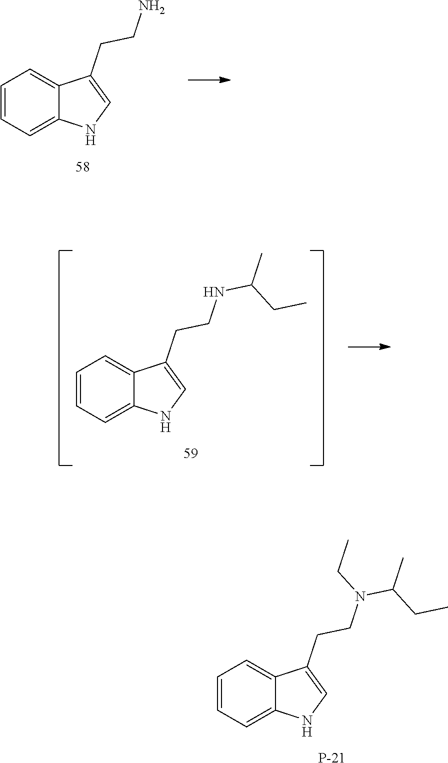

### Step 1: N-(2-(1H-indol-3-yl)ethyl)-N-ethylbutan-2-amine (P-21)

To a solution of tryptamine (250 mg, 1.56 mmol) in MeOH (12 mL) was added butan-2-one (337 mg, 4.68 mmol), NaBH₃CN (393 mg, 6.24 mmol) and AcOH (28 mg, 0.47 mmol). The reaction was stirred at ambient temperature for 3 h, and then acetaldehyde (274 mg, 6.24 mmol), NaBH₃CN (393 mg, 6.24 mmol), and AcOH (28 mg, 0.47 mmol) were added to the reaction. The mixture was stirred at ambient temperature for 1 h, quenched with H₂O (30 mL), and extracted with EtOAc (30 mL×2). The combined organic layer was washed with brine (40 mL), dried over Na₂SO₄ (15 g), filtered, and concentrated. The resulting residue was purified by flash silica chromatography (CH₂Cl₂-MeOH, 30:1 to 20:1) to afford the crude product (126 mg) which was dissolved in DCM (5 mL) and treated with HCl in Et2O (4 mL, 1 N) dropwise over 2 min, and stirred at ambient temperature for 30 min. The precipitate was then collected by filtration to afford N-(2-(1H-indol-3-yl)ethyl)-N-ethylbutan-2-amine as the hydrochloride salt (126 mg, 29%) which was an off-white solid. ¹H-NMR (300 MHz, DMSO-d₆) δ 11.01 (br s, 1H), 9.73 (br s, 1H), 7.61 (t, J=7.2 Hz, 1H), 7.37 (d, J=7.8 Hz, 1H), 7.30 (s, 1H), 7.11-6.98 (m, 2H), 3.51-3.12 (m, 7H), 1.98-1.82 (m, 1H), 1.61-1.48 (m, 1H), 1.38-1.20 (m, 6H), 1.00-0.89 (m, 3H). LCMS (ESI+): m/z 245.2 [M+H]⁺. HPLC Purity (220 nm): 99.8%.

Scheme 16: Compounds of general formula (I) can be synthesised from the appropriately substituted tryptamine following the outlined sequence of steps in Scheme 16 or similar as one skilled in the art may consider. An appropriately substituted tryptamine can undergo sequential reductive alkylations to access to compounds of general formula (I) (exemplified by P-22).

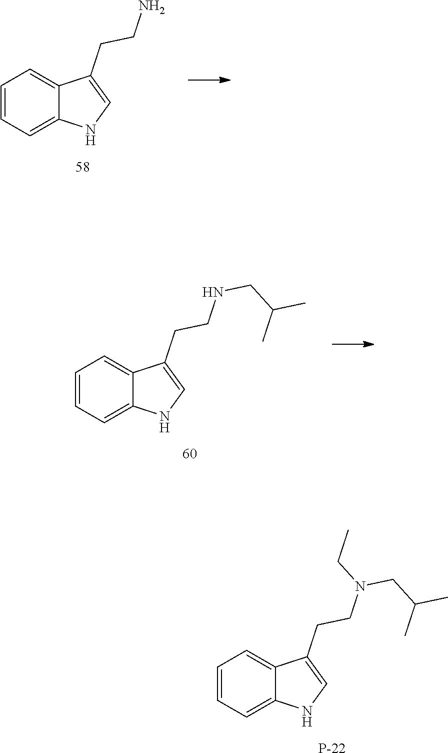

### Example 25: N-(2-(1H-indol-3-yl)ethyl)-N-ethyl-2-methylpropan-1-amine (P-22)

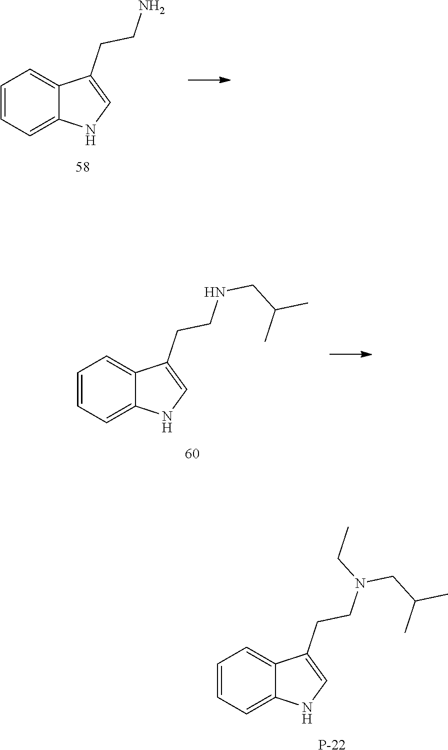

### Step 1: N-(2-(1H-indol-3-yl)ethyl)-2-methylpropan-1-amine (60)

To a solution of 2-(1H-indol-3-yl)ethan-1-amine (600 mg, 3.74 mmol) in MeOH (10 mL) was added isobutyraldehyde (283 mg, 3.93 mmol), NaBH₃CN (232 mg, 6.48 mmol), a catalytic amount AcOH (3 drops). The reaction was stirred at ambient temperature for 2 h, and then quenched with water (50 mL) and extracted with EtOAc (50 mL×2). The combined organic phase was washed with a saturated aqueous NaHCO₃ (50 mL) and brine (50 mL), dried over Na₂SO₄ (15 g), filtered, and concentrated. The resulting residue was purified by flash silica chromatography (CH₂Cl₂-MeOH 30:1 to 20:1) to give N-(2-(1H-indol-3-yl)ethyl)-2-methylpropan-1-amine (350 mg, 43% yield). LCMS (ESI+): m/z 217.4 [M+H]⁺.

### Step 2: N-(2-(1H-indol-3-yl)ethyl)-N-ethyl-2-methylpropan-1-amine (P-22)

To a solution of N-(2-(1H-indol-3-yl)ethyl)-2-methylpropan-1-amine (350 mg, 1.62 mmol) in MeOH (10 mL) was added acetaldehyde (285 mg, 6.48 mmol), NaBH₃CN (408 mg, 6.48 mmol) and AcOH (29 mg, 0.48 mmol). The reaction was stirred at ambient temperature for 2 h. After the reaction was completed as indicated by TLC, the mixture was quenched with water (30 mL) and extracted with EtOAc (30 mL×2). The combined organic layer was washed with brine (40 mL), dried over sodium sulfate (15 g), filtered and concentrated. The resulting residue was purified by flash silica chromatography (CH₂Cl₂-MeOH, 30:1 to 20:1) to give the crude product (170 mg) which was dissolved in DCM (3 mL) and treated with HCl in Et2O (4 mL, 1 N) dropwise over 2 min, and stirred at ambient temperature for 30 min. The reaction mixture was then concentrated and the residue was purified by preparative HPLC to afford N-(2-(1H-indol-3-yl)ethyl)-N-ethyl-2-methylpropan-1-amine as the hydrochoride salt (87 mg, 10%) which was an off-white solid. ¹H-NMR (300 MHz, DMSO-d₆): δ 11.01 (br s, 1H), 9.89 (br s, 1H), 7.65 (d, J=7.8 Hz, 1H), 7.37 (d, J=8.1 Hz, 1H), 7.27 (s, 1H), 7.15-6.97 (m, 2H), 3.32-3.12 (m, 6H), 3.09-2.92 (m, 2H), 2.12 (m, 1H), 1.31-1.20 (m, 3H), 1.08-0.98 (m, 6H). LCMS (ESI+): m/z 245.2 [M+H]⁺. HPLC Purity (220 nm): 98.7%.

Scheme 17: Compounds of general formula (I) can be synthesised from the appropriately substituted indole following the outlined sequence of steps in Scheme 17 or similar as one skilled in the art may. Substituted indole cores could be condensed with dimethylamino-2-nitroethylene to give nitrovinyl indoles. Reduction of such indoles proved viable in accessing unsubstituted ethyl amine analogues that could subsequently undergo sequential reductive alkylation reactions with to access compounds of general formula (I) (exemplified by P-23, P-24, P-25, and P-26).

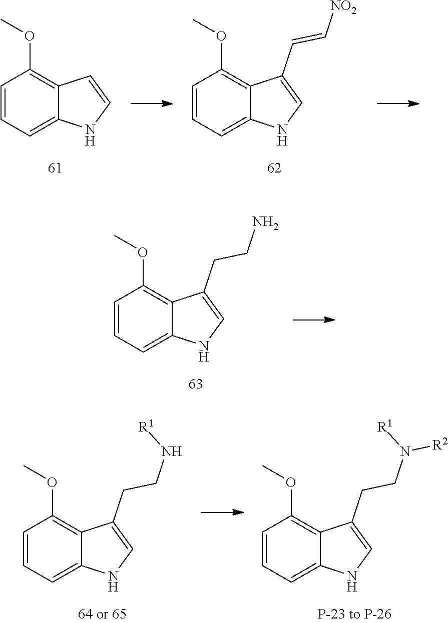

### Example 26: N-(2-(4-methoxy-1H-indol-3-yl)ethyl)-N-methylbutan-2-amine (P-23)

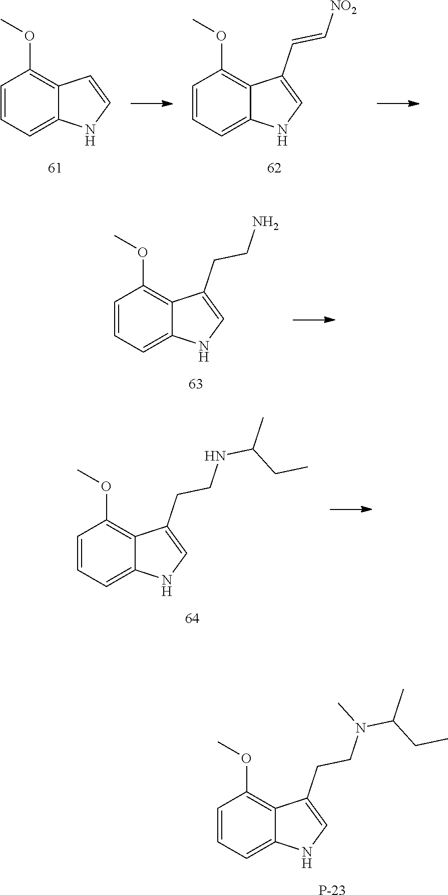

### Step 1: (E)-4-methoxy-3-(2-nitrovinyl)-1H-indole (62)

A mixture of 4-methoxy-1H-indole (15.0 g, 102 mmol), N,N-dimethyl-2-nitroethen-1-amine (11.8 g, 102 mmol) in TFA (105 mL) was degassed and purged with N₂ 3 times, and then the mixture was stirred at 25° C. for 1 h under N₂ atmosphere. The reaction mixture was quenched by addition to a rapidly stirring solution of saturated aqueous NaHCO₃ solution (105 mL×5), and resultant slurry was extracted with EtOAc (105 mL×5), dried over Na₂SO₄, filtered and concentrated under reduced pressure to give (E)-4-methoxy-3-(2-nitrovinyl)-1H-indole (16.0 g, 72% yield) as a red solid. ¹H NMR: (400 MHz, CDCl₃) δ 8.72-8.81 (m, 1H), 8.44-8.54 (m, 1H), 7.93-8.00 (m, 1H), 7.51-7.61 (m, 1H), 7.20-7.26 (m, 1H), 7.04 (d, J=8.0 Hz, 1H), 6.64-6.73 (m, 1H), 4.01-4.03 (m, 3H).

### Step 2: 2-(4-methoxy-1H-indol-3-yl)ethan-1-amine (63)

A mixture of LiAlH₄ (25.8 g, 680 mmol) in THF (47.0 mL) was degassed and purged with N₂ 3 times, and a solution of (E)-4-methoxy-3-(2-nitrovinyl)-1H-indole (13.5 g, 61.8 mmol) in THF (47.0 mL) was then added dropwise. Stirring was maintained at 0° C. for 1 h. Then the reaction was warmed to 25° C. and heated to 80° C. for 2 h. The reaction mixture was quenched by Na₂SO₄·10H₂O, filtered and concentrated under reduced pressure to give crude 2-(4-methoxy-1H-indol-3-yl)ethan-1-amine (8.00 g, 68% yield) that was used without further purification.

### Step 3: N-(2-(4-methoxy-1H-indol-3-yl)ethyl)butan-2-amine (64)

A solution of 2-(4-methoxy-1H-indol-3-yl)ethan-1-amine (0.50 g, 2.63 mmol), butan-2-one (284 mg, 3.94 mmol, 352 uL), and NEt₃ (1.33 g, 13.1 mmol, 1.83 mL) in DCE (25 mL) at 0° C. was treated with NaBH(OAc)₃ (2.79 g, 13.1 mmol). The mixture was stirred at 25° C. for 12 h. The residue was diluted with H₂O (20 mL) and extracted with EtOAc (10 mL×3), dried over Na₂SO₄, filtered and concentrated under reduced pressure to give N-(2-(4-methoxy-1H-indol-3-yl)ethyl)butan-2-amine (600 mg, 93%) as a brown solid which was used in the subsequent reactions without further purification.

### Step 3: N-(2-(4-methoxy-1H-indol-3-yl)ethyl)-N-methylbutan-2-amine (P-23)

A solution of N-(2-(4-methoxy-1H-indol-3-yl)ethyl)butan-2-amine (500 mg, 2.03 mmol), formaldehyde (91.4 mg, 3.04 mmol, 83.8 uL), and NEt₃ (1.03 g, 10.1 mmol, 1.41 mL) in DCE (20 mL) at 0° C. was treated with NaBH(OAc)₃ (2.15 g, 10.1 mmol). The mixture was stirred at 25° C. for 12 h. The residue was diluted with H₂O (30 mL) and extracted with EtOAc (20 mL×3), dried over Na₂SO₄, filtered and concentrated under reduced pressure to give a residue. The residue was purified by preparative HPLC (column: C18-1 150*30 mm, 5 um); mobile phase: [water(TFA)-ACN]; B %: 5%-50%, 8 min) to give N-(2-(4-methoxy-1H-indol-3-yl)ethyl)-N-methylbutan-2-amine (50.0 mg, 9.5% yield) as a yellow solid. ¹H NMR: (400 MHz, CDCl₃) δ 11.42-11.50 (br. s, 1H), 8.26-8.32 (br. s, 1H), 7.12 (t, J=8.0 Hz, 1H), 7.00 (m, 2H), 6.51 (d, J=8.0 Hz, 1H), 3.94 (s, 3H) 3.28-3.45 (m, 4H), 3.15-3.25 (m, 1H), 2.68-2.87 (m, 3H), 1.91-2.10 (m, 1H), 1.40-1.57 (m, 1H), 1.24-1.37 (m, 3H), 0.95-1.06 (m, 3H). LCMS (ESI+): m/z 261.1 [M+H]⁺. HPLC Purity (220 nm): 95.5%

### Example 27: N-ethyl-N-(2-(4-methoxy-1H-indol-3-yl)ethyl)butan-2-amine (P-24)

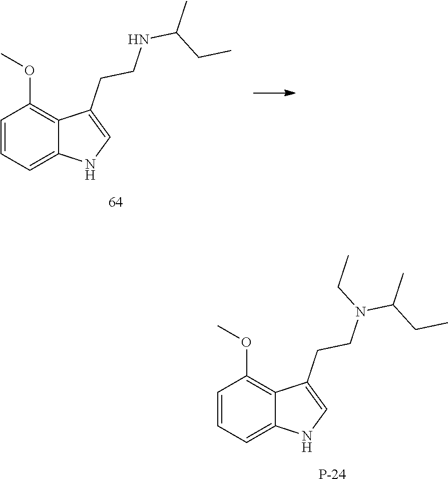

### Step 1: N-ethyl-N-(2-(4-methoxy-1H-indol-3-yl)ethyl) butan-2-amine (P-24)

A solution of N-(2-(4-methoxy-1H-indol-3-yl)ethyl)butan-2-amine (1.00 g, 4.06 mmol), acetaldehyde (268 mg, 6.09 mmol, 341 uL), and NEt₃ (2.05 g, 20.3 mmol, 2.83 mL) in DCE (40 mL) at 0° C. was treated with NaBH(OAc)₃ (4.30 g, 20.3 mmol). The mixture was stirred at 25° C. for 12 h. The residue was diluted with H₂O (15 mL) and extracted with EtOAc (10 mL×3), dried over Na₂SO₄, filtered and concentrated under reduced pressure to give a residue. The residue was purified by preparative HPLC (column: Phenomenex luna C18 250*50 mm, 10 um; mobile phase: [water(TFA)-ACN]; B %: 10%-40%, 10 min) to give N-ethyl-N-(2-(4-methoxy-1H-indol-3-yl)ethyl)butan-2-amine (0.33 g, 30% yield) as the trifluoroacetate salt which was a yellow solid. ¹H NMR (400 MHz, CDCl₃): δ 10.39-10.74 (br., 1H), 8.34 (br. s, 1H), 7.11 (t, J=8.0 Hz, 1H), 7.01 (m, 2H), 6.52 (d, J=7.6 Hz, 1H), 3.93 (s, 3H), 3.29-3.55 (m, 4H), 3.11-3.28 (m, 3H), 1.92-2.09 (m, 1H), 1.48-1.57 (m, 1H), 1.41-1.47 (t, J=7.2 Hz, 3H), 1.31-1.39 (m, 3H), 0.96-1.06 (m, 3H). LCMS (ESI+): m/z 274.1 [M+H]⁺. HPLC Purity (220 nm): 95.5%.

### Example 28: N-ethyl-N-(2-(4-methoxy-1H-indol-3-yl)ethyl)propan-2-amine (P-25)

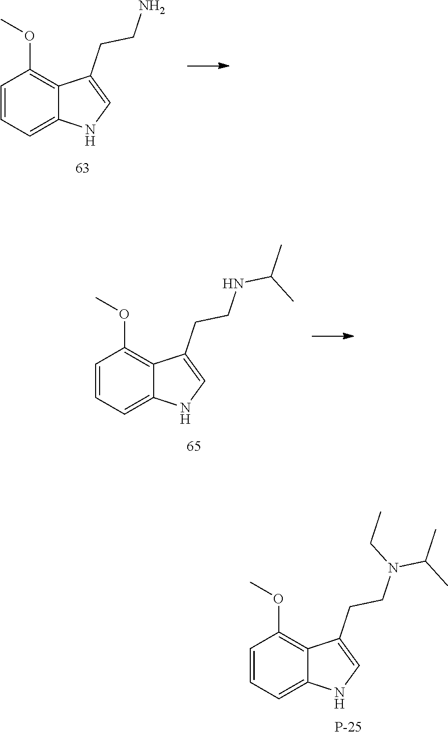

### Step 1: N-(2-(4-methoxy-1H-indol-3-yl)ethyl)propan-2-amine (65)

To a solution of 2-(4-methoxy-1H-indol-3-yl)ethan-1-amine (1.00 g, 5.26 mmol) in NEt₃ (2.66 g, 26.3 mmol, 3.66 mL) was added acetone (458 mg, 7.88 mmol, 580 uL), and the cooled (0° C.) solution was treated with NaBH(OAc)₃ (5.57 g, 26.3 mmol). The mixture was stirred at 25° C. for 12 h. The reaction mixture was diluted with H₂O (10 mL) and extracted with EtOAc (10 mL). The organic layers were concentrated under reduced pressure to give N-(2-(4-methoxy-1H-indol-3-yl)ethyl)propan-2-amine (980 mg, 80% yield) as a brown oil.

### Step 2: N-ethyl-N-(2-(4-methoxy-1H-indol-3-yl)ethyl)propan-2-amine (P-25)

To a solution of N-(2-(4-methoxy-1H-indol-3-yl)ethyl)propan-2-amine (780 mg, 3.36 mmol) in NEt₃ (1.70 g, 16.8 mmol, 2.34 mL) was added acetaldehyde (222 mg, 5.04 mmol, 283 uL), and the cooled (0° C.) solution was treated with NaBH(OAc)₃ (3.56 g, 16.8 mmol). The mixture was stirred at 25° C. for 12 h. The reaction mixture was diluted with H₂O (30 mL) and extracted with EtOAc (30 mL). The organic layers were concentrated under reduced pressure to give a residue. The crude product was purified by preparative HPLC (column: Phenomenex luna C18 250×50 mm×10 μm; mobile phase: [water(TFA)-ACN]; B: 10-40%, 10 min) to give N-ethyl-N-(2-(4-methoxy-1H-indol-3-yl)ethyl)propan-2-amine (40.0 mg, 4% over 2 steps) as the trifluoroacetate salt which was a yellow oil. ¹H NMR (400 MHz, CDCl₃) δ 11.20-11.36 (m, 1H), 8.01-8.20 (m, 1H), 7.09-7.16 (m, 1H), 6.99-7.05 (m, 2H), 6.47-6.58 (m, 1H), 3.89-4.02 (m, 3H), 3.73-3.85 (m, 1H), 3.14-3.41 (m, 6H), 1.44-1.48 (m, 3H), 1.41-1.44 (m, 3H), 1.36-1.39 (m, 3H) LCMS (ESI+): m/z 261.1 [M+H]⁺. HPLC Purity (220 nm): 98.6%.

### Example 29: N-isopropyl-N-(2-(4-methoxy-1H-indol-3-yl)ethyl)propan-1-amine (P-26)

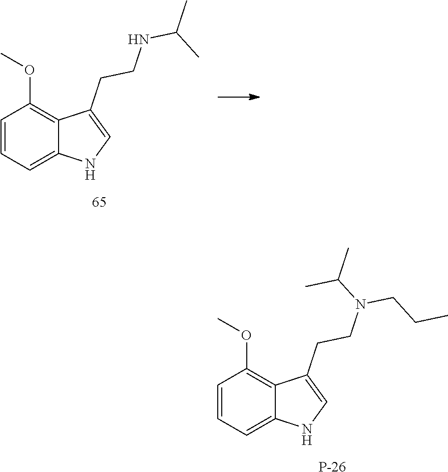

### Step 1: N-isopropyl-N-(2-(4-methoxy-1H-indol-3-yl)ethyl)propan-1-amine (P-26)

To a solution of N-(2-(4-methoxy-1H-indol-3-yl)ethyl)propan-2-amine (930 mg, 4.00 mmol) in NEt₃ (2.03 g, 20.0 mmol, 2.79 mL) was added propanal (349 mg, 6.00 mmol, 437 μL), and the cooled (0° C.) solution was treated with NaBH(OAc)₃ (4.24 g, 20.0 mmol). The mixture was stirred at 25° C. for 12 h. The reaction mixture was diluted with H₂O (30.0 mL) and extracted with EtOAc (30.0 mL). The organic layers were concentrated under reduced pressure to give a residue which was purified by preparative HPLC (column: Phenomenex luna C18 250×50 mm×10 μm; mobile phase: [water(TFA)-ACN]; B: 10-40%, 10 min) to give N-isopropyl-N-(2-(4-methoxy-1H-indol-3-yl)ethyl)propan-1-amine (70.0 mg, 5.8% yield) as the trifluoroacetate salt which was a yellow oil. ¹H NMR (400 MHz, CDCl₃): δ 11.20-11.10 (m, 1H), 8.87-8.80 (m, 1H), 7.12-7.06 (m, 1H), 7.04-6.99 (m, 1H), 6.97-6.93 (m, 1H), 6.54-6.44 (m, 1H), 3.91 (s, 3H), 3.75-3.72 (m, 1H), 3.35-3.14 (m, 4H), 3.00-2.95 (m, 2H), 1.95-1.81 (m, 2H), 1.40-1.01 (m, 6H), 0.94-1.06 (m, 3H). LCMS (ESI+): m/z 275.1 [M+H]⁺. HPLC Purity (220 nm): 99.5%.

Scheme 18: Compounds of general formula (I) can be synthesised from the appropriately substituted tryptamine following the outlined sequence of steps in Scheme 18 or similar as one skilled in the art may consider. The substituted tryptamine 63 can be derivatised to the tert-butyl carbamate 238 which can be chemoselectively reduced to provide access to N-methylated tryptamine 239. Subsequent reductive alkylation provides access to compounds of general formula (I) (exemplified by P-136).

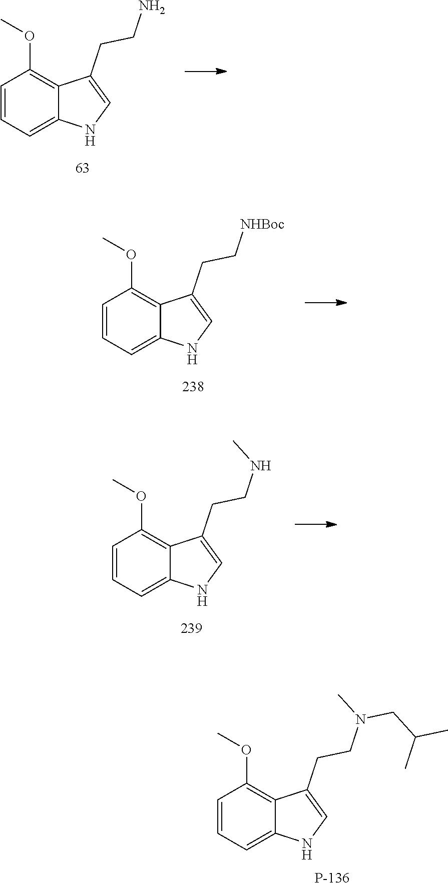

### Example 30: N-(2-(4-methoxy-1H-indol-3-yl)ethyl)-N,2-dimethylpropan-1-amine (P-136)

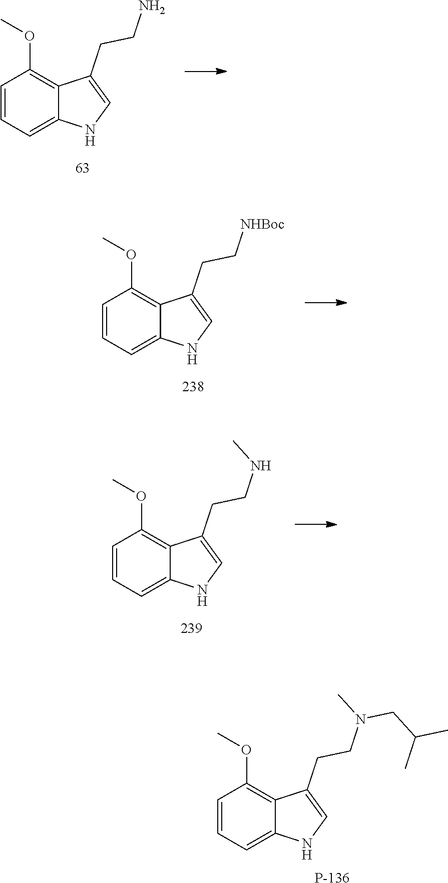

### Step 1: tert-butyl (2-(4-methoxy-1H-indol-3-yl)ethyl)carbamate (238)

A solution of 2-(4-methoxy-1H-indol-3-yl)ethan-1-amine (2.00 g, 10.5 mmol) and Boc₂O (2.41 g, 11.0 mmol) in THF (14 mL) was degassed and purged with N₂ three times and then stirred at ambient temperature for 2 h under N₂. Upon completion, the reaction mixture was diluted with H₂O (25 mL) and extracted with EtOAc (15 mL×3). The organic layer was dried over anhydrous Na₂SO₄ and concentrated to afford crude tert-butyl (2-(4-methoxy-1H-indol-3-yl)ethyl)carbamate (3.00 g) as a brown oil which was used in the next step without further purification.

### Step 2: 2-(4-methoxy-1H-indol-3-yl)-N-methylethan-1-amine (239)

A solution of tert-butyl (2-(4-methoxy-1H-indol-3-yl)ethyl)carbamate (1.00 g, 3.44 mmol) in THF (3.50 mL) was added to a stirring mixture of LiAlH₄ (653 mg, 17.2 mmol) in THF (3.50 mL), which had been degassed and purged with N₂ three times, at 0° C. The resulting mixture was heated at 70° C. for 3 h. Upon completion, the reaction was quenched by addition of Na₂SO₄·10H₂O and then filtered. The filtrate was concentrated in vacuo to afford crude 2-(4-methoxy-1H-indol-3-yl)-N-methylethan-1-amine (700 mg) as a brown oil which was used in the next step without further purification.

### Step 3: N-(2-(4-methoxy-1H-indol-3-yl)ethyl)-N,2-dimethylpropan-1-amine (P-136)

A solution of 2-(4-methoxy-1H-indol-3-yl)-N-methylethan-1-amine (600 mg) and 3-methylbutanal (379 mg, 4.41 mmol) in Et₃N (1.49 g, 14.7 mmol) was treated with NaBH(OAc)₃ (3.11 g, 14.7 mmol) at 0° C. The reaction mixture was stirred at ambient temperature for 12 h. Upon completion, the reaction mixture was diluted with H₂O (10 mL) and extracted with EtOAc (15 mL×3). The organic layer was dried over anhydrous Na₂SO₄ and concentrated in vacuo. The residue was purified by preparative HPLC (Column: C18-1 (150×30 mm×5 μm); Mobile phase: [water(TFA)-ACN); B: 10-55%, 8 min) to afford N-(2-(4-methoxy-1H-indol-3-yl)ethyl)-N,2-dimethylpropan-1-amine trifluoroacetate (56.0 mg, 1% over 3 steps) as a yellow solid. ¹H NMR (400 MHz, CDCl₃): δ 11.35-11.75 (br., 1H), 8.28 (br s, 1H), 7.12 (t, J=8.0 Hz, 1H), 7.00 (d, J=8.0 Hz, 1H), 6.97 (s, 1H), 6.52 (d, J=7.6 Hz, 1H), 3.94 (s, 3H), 3.37-3.50 (m, 1H), 3.23-3.36 (m, 3H), 2.97-3.05 (m, 1H), 2.77-2.96 (m, 4H), 2.04-2.22 (m, 1H), 1.08 (d, J=6.8 Hz, 6H). LCMS (ESI+): m/z 261.1 [M+H]⁺. HPLC Purity (220 nm): 100%

Scheme 19: Compounds of general formula (I) can be synthesised from the appropriately substituted tryptamine following the outlined sequence of steps in Scheme 19 or similar as one skilled in the art may consider. The substituted tryptamine 63 can be acetylated to give 240 before chemoselective reduction to provide access to N-ethylated tryptamine 241. Subsequent reductive alkylation provides access to compounds of general formula (I) (exemplified by P-137).

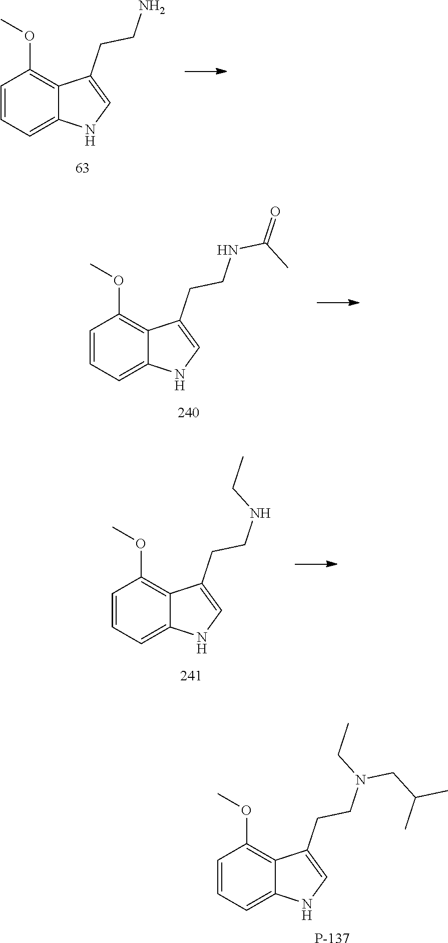

### Example 31: N-(2-(4-methoxy-1H-indol-3-yl)ethyl)-N,2-dimethylpropan-1-amine (P-137)

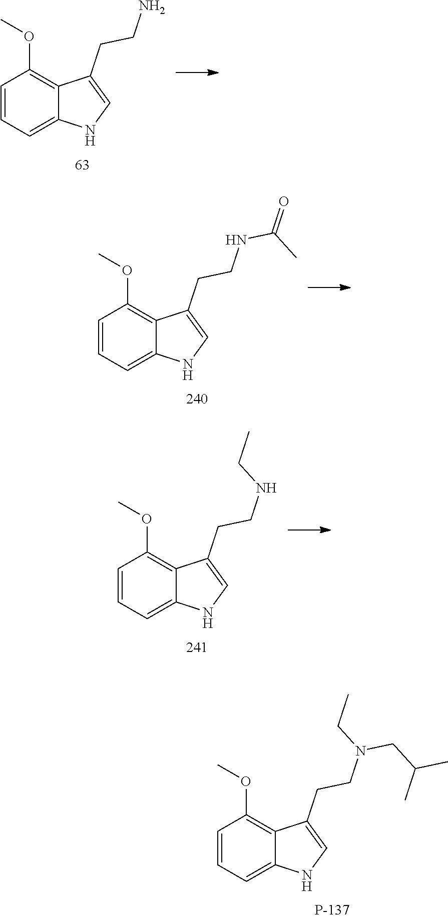 [SMILES: COc1cccc2[nH]cc(CCN)c12 ; COc1cccc2[nH]cc(CCNC(C)=O)c12 ; CCNCCc1c[nH]c2cccc(OC)c12 ; CCN(CCc1c[nH]c2cccc(OC)c12)CC(C)C]

### Step 1: N-(2-(4-methoxy-1H-indol-3-yl)ethyl)acetamide (240)

A solution of 2-(4-methoxy-1H-indol-3-yl)ethan-1-amine (2.00 g, 10.5 mmol) in pyridine (9.30 mL) was treated with Ac₂O (4.60 mL) and the reaction was stirred at ambient temperature for 12 h. The reaction was then concentrated in vacuo to afford a crude N-(2-(4-methoxy-1H-indol-3-yl)ethyl)acetamide (2.44 g) as a brown solid and was used in the next step without further purification.

### Step 2: N-ethyl-2-(4-methoxy-1H-indol-3-yl)ethan-1-amine (241)

A solution of crude N-(2-(4-methoxy-1H-indol-3-yl)ethyl)acetamide (2.30 g) and LiAlH₄ (1.88 g, 49.5 mmol) in THF (16.1 mL) was degassed and purged with N₂ three times at 0° C. and stirred at 0° C. for 10 min. The reaction was then allowed to warm to ambient temperature before being heated at 50° C. for 5 h under N₂. The reaction was quenched by addition of Na₂SO₄·10H₂O (2.00 g), filtered, and the filtrate concentrated in vacuo to afford crude N-ethyl-2-(4-methoxy-1H-indol-3-yl)ethan-1-amine (2.16 g) as a brown solid.

### Step 3: N-ethyl-N-(2-(4-methoxy-1H-indol-3-yl)ethyl)-2-methylpropan-1-amine (P-137)

A solution of crude N-ethyl-2-(4-methoxy-1H-indol-3-yl)ethan-1-amine (2.06 g) and isobutyraldehyde (1.02 g, 14.2 mmol) in Et₃N (4.77 g, 47.2 mmol) was treated NaBH(OAc)₃ (10.0 g, 47.2 mmol) at 0° C. The reaction was then stirred at ambient temperature for 12 h. The reaction was quenched with water (30 mL) and extracted with EtOAc (20 mL×3). The combined organic layer was dried over anhydrous Na₂SO₄ and concentrated in vacuo. The residue was purified by preparative HPLC to afford N-ethyl-N-(2-(4-methoxy-1H-indol-3-yl)ethyl)-2-methylpropan-1-amine trifluoroacetate (2.00 g, 49% over 3 steps) as a yellow oil. ¹H NMR (400 MHz, CDCl₃): δ 11.10 (br. s, 1H), 8.30 (br. s, 1H), 7.12 (t, J=7.8 Hz, 1H), 7.03-6.97 (m, 2H), 6.52 (d, J=7.8 Hz, 1H), 3.93 (s, 3H), 3.38-3.25 (m, 6H), 2.93 (m, 2H), 2.15 (m, 1H), 1.38 (t, J=7.2 Hz, 3H), 1.08 (d, J=6.4 Hz, 6H). LCMS (ESI+): m/z 275.1 [M+H]⁺. HPLC Purity (220 nm): 98.8%

### Example 32: 3-(2-(ethyl(isopropyl)amino)ethyl)-1H-indol-4-ol (P-138)

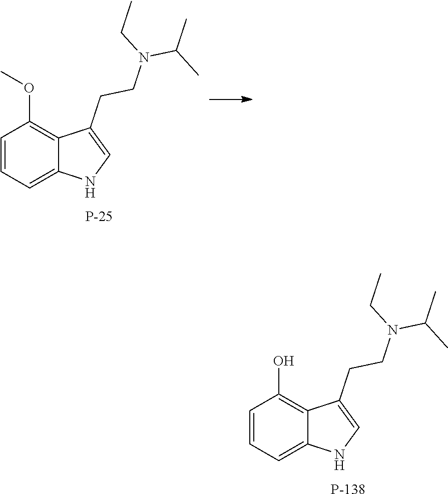

### Step 1: 3-(2-(ethyl(isopropyl)amino)ethyl)-1H-indol-4-ol (P-138)

A solution of N-ethyl-N-(2-(4-methoxy-1H-indol-3-yl)ethyl)propan-2-amine (400 mg, 1.54 mmol) in CH₂Cl₂ (2.80 mL) was treated with AlCl₃ (1.23 g, 9.22 mmol) and EtSH (1.72 g, 27.7 mmol) at 0° C. The resulting mixture was stirred at ambient temperature for 1 h. Upon completion, the reaction was cooled to 0° C. and quenched by addition of saturated aqueous NaHCO₃ (15 mL) and extracted with CH₂Cl₂ (10 mL×3). The combined organics were washed with brine (30 mL), dried over anhydrous Na₂SO₄ and concentrated in vacuo. The residue was purified by preparative HPLC (Column: C18-1 (150×30 mm×5 μm); mobile phase: [water(TFA)-ACN]; B: 1-25%, 8 min) to afford 3-(2-(ethyl(isopropyl)amino)ethyl)-1H-indol-4-ol trifluoroacetate (16.9 mg, 3%) as a brown oil. ¹H NMR (400 MHz, MeOD₄) δ 7.03 (s, 1H), 6.90 (m, 2H), 6.37 (d, J=7.6 Hz, 1H), 3.75 (m, 1H), 3.60 (m, 1H), 3.24-3.37 (m, 5H), 1.36 (m, 9H). LCMS (ESI+): m/z 247.1 [M+H]⁺. HPLC Purity (220 nm): 100%

### Example 33: 3-(2-(sec-butyl(ethyl)amino)ethyl)-1H-indol-4-ol (P-139)

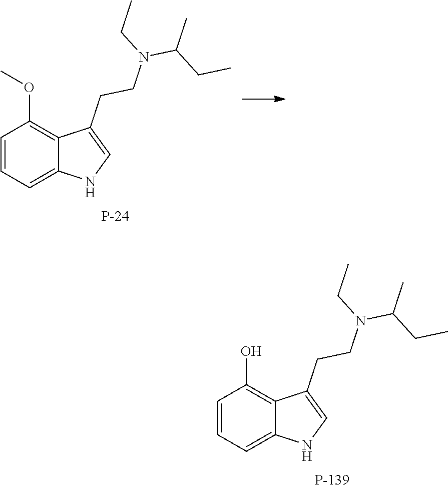

### Step 1: 3-(2-(sec-butyl(ethyl)amino)ethyl)-1H-indol-4-ol (P-139)

A solution of N-ethyl-N-(2-(4-methoxy-1H-indol-3-yl)ethyl)butan-2-amine (875 mg, 2.25 mmol) in CH₂Cl₂ (4.2 mL) was treated with AlCl₃ (2.7 g, 9 eq., 20.3 mmol) and EtSH (2.52 g, 18 eq., 40.5 mmol) at 0° C. The resulting mixture was purged with N₂ three times and stirred at ambient temperature for 1 h. The reaction was cooled to 0° C. and quenched by addition of saturated aq. NaHCO₃ solution (15 mL) and then extracted with CH₂Cl₂ (10 mL×3). The combined organic layers were washed with brine (30 mL), dried over anhydrous Na₂SO₄, filtered, and the filtrate concentrated in vacuo. The residue was purified by preparative HPLC (column: C18-1, 150×30 mm×5 μm; mobile phase: [water (TFA)-ACN]; B %: 5%-50%, 8 min) to afford 3-(2-(sec-butyl(ethyl)amino)ethyl)-1H-indol-4-ol as the trifluoroacetate salt (9.4 mg, 1%) which was a yellow oil. ¹H NMR (400 MHz, MeOD-d₄): δ 7.03 (s, 1H), 6.83-6.93 (m, 2H), 6.37 (d, J=7.2 Hz, 1H), 3.31-3.48 (m, 4H), 3.24-3.30 (m, 3H), 1.75-1.83 (m, 1H), 1.55-1.68 (m, 1H), 1.24-1.42 (m, 6H), 0.93-1.05 (m, 3H). LCMS (ESI+): m/z 261.1 [M+H]⁺. HPLC Purity (220 nm): 96.7%.

### Example 34: 3-(2-(ethyl(isobutyl)amino)ethyl)-1H-indol-4-ol (P-140)

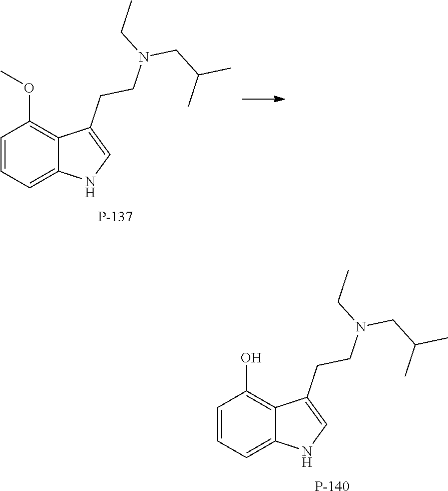

### Step 1: 3-(2-(ethyl(isobutyl)amino)ethyl)-1H-indol-4-ol (P-140)

A solution of N-ethyl-N-(2-(4-methoxy-1H-indol-3-yl)ethyl)-2-methylpropan-1-amine (0.8 g, 2.92 mmol) in CH₂Cl₂ (5 mL) was treated with AlCl₃ (3.5 g, 9 eq., 26.2 mmol) and EtSH (3.26 g, 18 eq., 52.5 mmol) at 0° C. The resulting mixture was purged with N₂ three times and stirred at ambient temperature for 1 h. The reaction was cooled to 0° C. and quenched by addition of saturated aq. NaHCO₃ solution (15 mL) and then extracted with CH₂Cl₂ (10 mL×3). The combined organic layers were washed with brine (30 mL), dried over anhydrous Na₂SO₄, filtered, and the filtrate concentrated in vacuo. The residue was purified by preparative HPLC (column: C18-1 150*30 mm*5 μm; mobile phase: [water (TFA)-ACN]; B %: 1%-45%, 8 min) to afford 3-(2-(ethyl(isobutyl)amino)ethyl)-1H-indol-4-ol as the trifluoroacetate salt (38.2 mg, 4%) which was a yellow oil. ¹H NMR (400 MHz, MeOD-d₄): δ 7.03 (s, 1H), 6.94-6.83 (m, 2H), 6.38 (d, J=7.2 Hz, 1H), 3.64-3.41 (m, 2H), 3.38-3.29 (m, 4H), 3.10 (m, 1H), 2.99 (m, 1H), 2.15 (m, 1H), 1.38 (t, J=7.2 Hz, 3H), 1.05-1.00 (m, 6H). LCMS (ESI+): m/z 261.1 [M+H]⁺. HPLC Purity (220 nm): 97.7%.

### Example 35: 3-(2-(isopropyl(propyl)amino)ethyl)-1H-indol-4-ol (P-141)

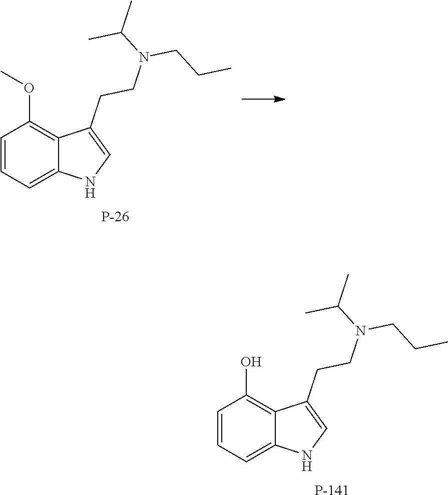

### Step 1: 3-(2-(isopropyl(propyl)amino)ethyl)-1H-indol-4-ol (P-141)

A solution of N-isopropyl-N-(2-(4-methoxy-1H-indol-3-yl)ethyl)propan-1-amine (0.5 g, 1.82 mmol) in CH₂Cl₂ (3.5 mL) was treated with AlCl₃ (1.46 g, 6 eq., 10.9 mmol) and EtSH (2.04 g, 18 eq., 32.8 mmol) at 0° C. The resulting mixture was purged with N₂ three times and stirred at ambient temperature for 1 h. The reaction was cooled to 0° C. and quenched by addition of saturated aq. NaHCO₃ solution (20 mL) and then extracted with CH₂Cl₂ (20 mL×3). The combined organic layers were washed with brine (50 mL), dried over anhydrous Na₂SO₄, filtered, and the filtrate concentrated in vacuo. The residue was purified by preparative HPLC (column: C18-1 150*30 mm*5 μm; mobile phase: [water (TFA)-ACN]; B %: 5%-50%, 8 min) to afford 3-(2-(isopropyl(propyl)amino)ethyl)-1H-indol-4-ol as the trifluoroacetate salt (230 mg, 34%) which was a brown oil. ¹H NMR (400 MHz, MeOD-d₄): δ 7.04 (s, 1H), 6.85-6.94 (m, 2H), 6.38 (dd, J=7.4, 0.8 Hz, 1H), 3.71-3.81 (m, 1H), 3.56-3.65 (m, 1H), 3.35-3.40 (m, 1H), 3.24-3.29 (m, 2H), 3.08-3.20 (m, 2H), 1.72-1.90 (m, 2H), 1.36 (d, J=6.4 Hz, 6H), 1.02 (t, J=7.2 Hz, 3H). LCMS (ESI+): m/z 261.1 [M+H]⁺. HPLC Purity (220 nm): 100%.

Scheme 20: Compounds of general formula (I) can be synthesised from the appropriately substituted indole following the outlined sequence of steps in Scheme 20 or similar as one skilled in the art may consider. Amidation of the pendant acetic acid with an appropriately substituted secondary amine provided access to intermediates such as 301 which when subjected to reductive conditions allowed access to the desired compounds of general formula (I) exemplified by P-137. One skilled in the art will recognise that utilising differentially substituted amines would allow access to compounds of general formula (I) disclosed herein.

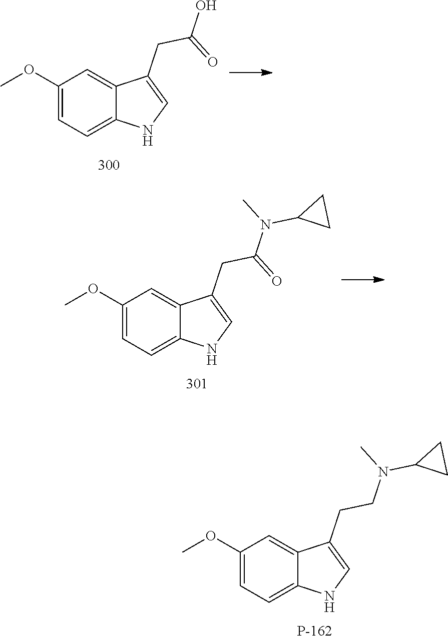

### Example 36: N-(2-(5-methoxy-1H-indol-3-yl)ethyl)-N-methylcyclopropanamine (P-162)

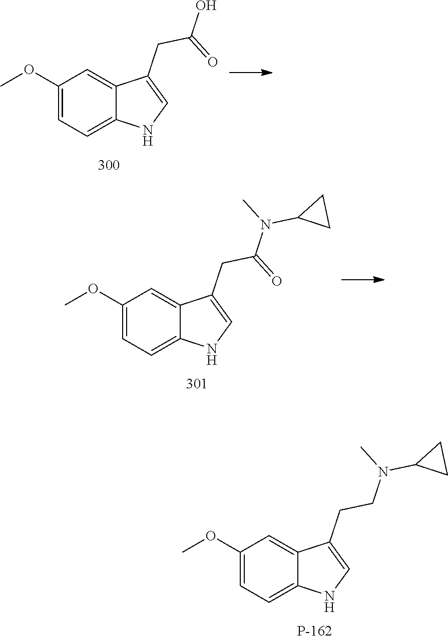

### Step 1: N-cyclopropyl-2-(5-methoxy-1H-indol-3-yl)-N-methylacetamide (301)

To a solution of 2-(5-methoxy-1H-indol-3-yl)acetic acid (500 mg, 2.44 mmol, 1.0 equiv.) in CH₂Cl₂ (5 mL) was added Et₃N (370 mg, 3.65 mmol, 509 μL, 1.5 equiv.), HATU (1.39 g, 3.65 mmol, 1.5 equiv.) and N-methylcyclopropanamine (173 mg, 2.44 mmol, 1.0 equiv.), and the mixture stirred at 25° C. for 2 h. The reaction mixture was diluted with H₂O (10 mL) and extracted with EtOAc (15 mL×3). The combined organic layers were washed with brine (10 mL×2), dried over Na₂SO₄, filtered, and concentrated under reduced pressure to give crude N-cyclopropyl-2-(5-methoxy-1H-indol-3-yl)-N-methylacetamide (500 mg) as a yellow oil which was used in the subsequent step without further purification. LCMS (ESI+): m/z 259.0 [M+H]⁺.

### Step 2: N-(2-(5-methoxy-1H-indol-3-yl)ethyl)-N-methylcyclopropanamine (P-162)

A cooled (0° C.) solution of crude N-cyclopropyl-2-(5-methoxy-1H-indol-3-yl)-N-methylacetamide (300 mg) in THF (3.00 mL) was degassed and purged with N₂ 3 times, and then treated dropwise with a solution of LiAlH₄ in THF (2.50 M, 2.32 mL). The mixture was stirred at 25° C. for 3 h under N₂, and then cooled to 0° C. and quenched by addition Na₂SO₄·10H₂O (3.00 g). The mixture was filtered, the filter cake was washed with EtOAc (15 mL×3), and the combined organic phases were concentrated under reduced pressure. The residue was purified by preparative HPLC (neutral condition: column: Waters Xbridge 150×25 mm×5 μm; mobile phase: [water (NH₄HCO₃)—ACN]; gradient: 35%-65% B over 9 min) to give N-(2-(5-methoxy-1H-indol-3-yl)ethyl)-N-methylcyclopropanamine (25.0 mg, 4% over 2 steps) as a yellow solid. ¹H NMR (400 MHz, CDCl₃): δ 7.86 (br s, 1H), 7.26-7.24 (m, 1H), 7.07 (d, J=2.4 Hz, 1H), 7.01 (d, J=2.0 Hz, 1H), 6.87 (dd, J=8.8, 2.4 Hz, 1H), 3.88 (s, 3H), 3.01-2.85 (m, 4H), 2.49 (s, 3H), 1.74-1.73 (m, 1H), 0.56-0.52 (m, 4H); LCMS (ESI+): m/z 245.0 [M+H]⁺; HPLC purity (220 nm): 99.6%.

### Example 37: N-(2-(5-methoxy-1H-indol-3-yl)ethyl)-N-methylcyclobutanamine (P-163)

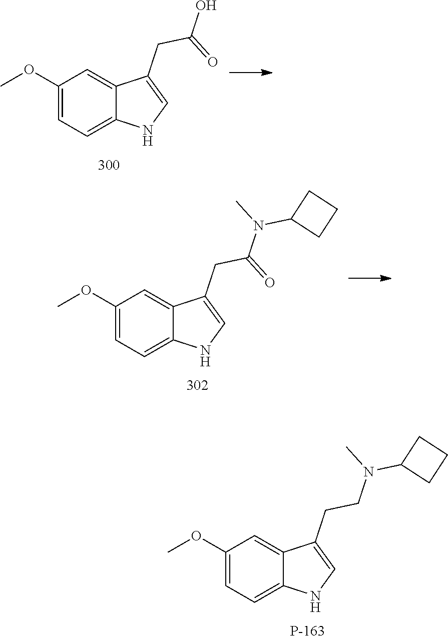

### Step 1: N-cyclobutyl-2-(5-methoxy-1H-indol-3-yl)-N-methylacetamide (302)

To a solution of 2-(5-methoxy-1H-indol-3-yl)acetic acid (0.50 g, 2.44 mmol, 1.0 equiv.) and N-methylcyclobutanamine (249 mg, 2.92 mmol, 1.2 equiv.) in CH₂Cl₂ (10 mL) was added Et₃N (370 mg, 3.65 mmol, 509 μL, 1.5 equiv.) and HATU (1.39 g, 3.65 mmol, 1.5 equiv.) and the mixture was stirred at 25° C. for 2 h. The reaction mixture was washed with H₂O (10 mL×2), brine (10 mL), dried over Na₂SO₄, concentrated in vacuo to give crude N-cyclobutyl-2-(5-methoxy-1H-indol-3-yl)-N-methylacetamide (0.70 g) as a pale brown oil which was used in the subsequent step without further purification. LCMS (ESI+): m/z 273.0 [M+H]⁺.

### Step 2: N-(2-(5-methoxy-1H-indol-3-yl)ethyl)-N-methylcyclobutanamine (P-163)

A cooled (0° C.) solution of crude N-cyclobutyl-2-(5-methoxy-1H-indol-3-yl)-N-methylacetamide (700 mg) in THF (7 mL) was degassed and purged with N₂ 3 times, and then treated dropwise with a solution of LiAlH₄ in THF (2.50 M, 5.14 mL), and stirred at 25° C. for 3 h. The reaction mixture was cooled to 0° C., and quenched by the addition Na₂SO₄—H₂O (7.50 g). The mixture was filtered, the filter cake was washed with EtOAc (30 mL×3), and the combined organic phases were concentrated under reduced pressure. The residue was purified by preparative HPLC [column: Waters Xbridge Prep OBD C18 150×40 mm×10 μm; mobile phase: [water (NH₄HCO₃)—ACN]; gradient: 20%-50% B over 15 mins] to give N-(2-(5-methoxy-1H-indol-3-yl)ethyl)-N-methylcyclobutanamine (259 mg, 41% over 2 steps) as a yellow solid. ¹H NMR (400 MHz, MeOD-d4): δ 7.22 (d, J=8.8 Hz, 1H), 7.03-6.98 (m, 2H), 6.75 (dd, J=8.8, 2.4 Hz, 1H), 3.82 (s, 3H), 3.06-2.95 (m, 1H), 2.94-2.87 (m, 2H), 2.69-2.61 (m, 2H), 2.30 (s, 3H), 2.16-2.07 (m, 2H), 2.03-1.91 (m, 2H), 1.78-1.64 (m, 2H); LCMS (ESI+): m/z 259.0 [M+H]⁺; HPLC purity (254 nm): 98.4%.

### Example 38: N-(2-(5-methoxy-1H-indol-3-yl)ethyl)-N-methyloxetan-3-amine (P-164)

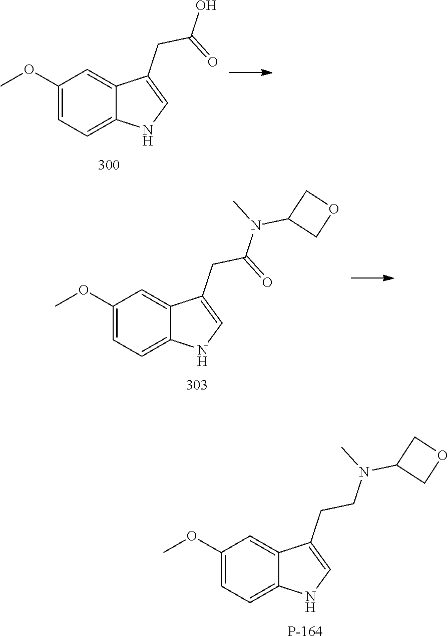

### Step 1: 2-(5-methoxy-1H-indol-3-yl)-N-methyl-N-(oxetan-3-yl)acetamide (303)

To a solution of 2-(5-methoxy-1H-indol-3-yl)acetic acid (500 mg, 2.44 mmol, 1.0 equiv.) and N-methyloxetan-3-amine (254 mg, 2.92 mmol, 1.2 equiv.) in CH₂Cl₂ (10 mL) was added Et₃N (370 mg, 3.65 mmol, 509 μL, 1.5 equiv.), HATU (1.39 g, 3.65 mmol, 1.5 equiv.), and the mixture was stirred at 25° C. for 2 h. The reaction mixture was washed with H₂O (10 mL×2), brine (10 mL), dried over Na₂SO₄, filtered, and the filtrate concentrated in vacuo to give crude 2-(5-methoxy-1H-indol-3-yl)-N-methyl-N-(oxetan-3-yl)acetamide (0.70 g) was obtained as a brown oil which was used in the subsequent step without further purification. LCMS (ESI+): m/z 275.2 [M+H]⁺.

### Step 2: N-(2-(5-methoxy-1H-indol-3-yl)ethyl)-N-methyloxetan-3-amine (P-164)

A cooled (0° C.) solution of crude 2-(5-methoxy-1H-indol-3-yl)-N-methyl-N-(oxetan-3-yl)acetamide (0.70 g) in THF (10 mL) was treated dropwise with a solution of LiAlH₄ in THF (2.50 M, 5.10 mL) under N₂, and the mixture was stirred at 25° C. for 5 h. The reaction mixture was cooled to 0° C., quenched with Na₂SO₄·10H₂0 (10.0 g), and stirred for 30 min. The mixture was filtered, the filter cake was washed with EtOAc (20 mL×5), and the combined organic layers were concentrated in vacuo. The residue was purified by preparative HPLC (column: Waters Xbridge Prep OBD C18 150×40 mm×10 μm; mobile phase: [water (NH₄HCO₃)—ACN]; gradient: 15-45% B over 15 mins) to give N-(2-(5-methoxy-1H-indol-3-yl)ethyl)-N-methyloxetan-3-amine (20.0 mg, 3% over 2 steps) as a yellow solid. ¹H NMR (400 MHz, CDCl₃): δ 7.96 (br s, 1H), 7.29 (s, 1H), 7.06-7.04 (m, 2H), 6.90 (dd, J=8.8, 2.4 Hz, 1H), 4.68 (d, J=6.8 Hz, 4H), 3.90 (s, 3H), 3.72-3.63 (m, 1H), 2.95-2.85 (m, 2H), 2.63-2.53 (m, 2H), 2.31 (s, 3H); LCMS (ESI+): m/z 261.0 [M+H]⁺; HPLC purity (220 nm): 99.5%.

Scheme 21: Compounds of general formula (I) can be synthesised from the appropriately substituted tryptamine following the outlined sequence of steps in Scheme 21 or similar as one skilled in the art may consider. Installation of a carbamate onto the pendant amine followed by reduction generates mono-methyl analogue 402. This intermediate can be condensed with various aldehydes or ketones under reductive alkylation conditions to generate compounds of general formula (I) exemplified by compound P-42. One skilled in the art will recognise that utilising alternate aldehydes and ketones would allow access to alternate compounds of general formula (I) disclosed herein.

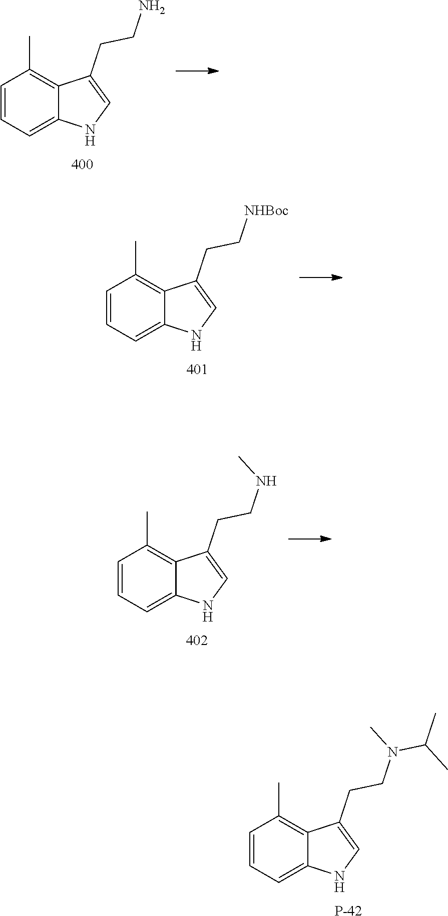

### Example 39: N-methyl-N-(2-(4-methyl-1H-indol-3-yl)ethyl)propan-2-amine (P-42)

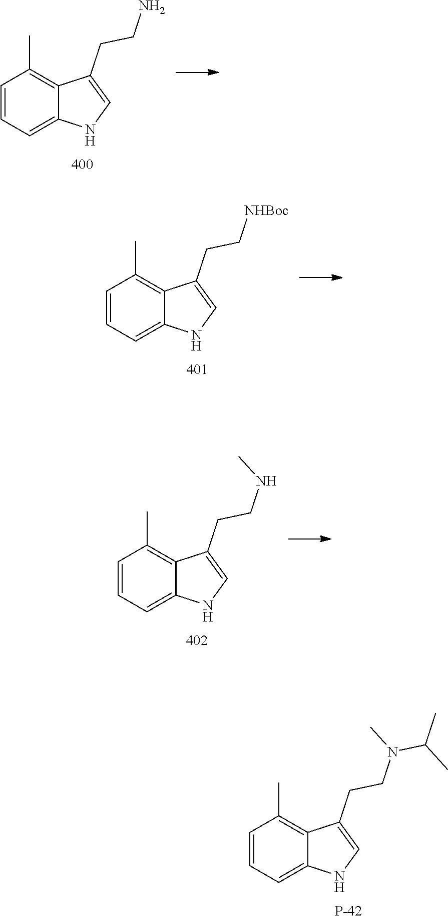

### Step 1: tert-butyl (2-(4-methyl-1H-indol-3-yl)ethyl)carbamate (401)

To an ice-cold solution of 2-(4-methyl-1H-indol-3-yl)ethan-1-amine (2.0 g, 11.5 mmol) in THF (100 mL, 1.23 mol) was added triethylamine (2.4 mL, 1.5 eq., 17.2 mmol) and Boc₂0 (3.01 g, 1.2 eq., 13.8 mmol), pre-dissolved in 20 mL of THF. The reaction was stirred at ambient temperature for 16 h and then diluted with cold water (50 mL). The solvent volume reduced by half under a stream of N₂ gas. The resulting solution was then extracted with Et₂O (50 mL×3), and the combined organic layers were washed with 0.1 M aq. HCl (50 mL×3) and brine (100 mL×1). The organic layer was concentrated in vacuo to afford a solid which was then triturated with hexane and filtered. The collected solid was washed with additional hexane (10 mL×1), to afford tert-butyl (2-(4-methyl-1H-indol-3-yl)ethyl)carbamate, after drying, as a red solid (2.9 g, 92%). ¹H NMR (400 MHz, DMSO-d₆): δ 10.77 (s, 1H), 7.14 (d, J=8.0 Hz, 1H), 7.06 (s, 1H), 6.95-6.86 (m, 2H), 6.68 (d, J=7.0 Hz, 1H), 3.25-3.14 (m, 2H), 2.95 (t, J=7.6 Hz, 2H), 2.61 (s, 3H), 1.39 (s, 9H).

### Step 2: N-methyl-2-(4-methyl-1H-indol-3-yl)ethan-1-amine (402)

To an ice-cold solution of anhydrous THF (100 mL) was added LiAlH₄ (1.16 g, 3 eq., 30.6 mmol) in portions under N₂, followed by tert-butyl (2-(4-methyl-1H-indol-3-yl)ethyl)carbamate (2.8 g, 10.2 mmol), pre-dissolved in minimal anhydrous THF, at a rate that maintained a gentle reflux. After the addition, the reaction was heated to reflux for 1 hour. The completed reaction was cooled in an ice bath and quenched with cold water (1.2 mL), 3.75 M aq. NaOH (1.2 mL), and water (3.6 mL). The resulting suspension was stirred cold for 15 minutes, then dried with ˜1 g of anhydrous Na₂SO₄, filtered through a pad of Celite, and the filter cake was washed with several volumes of hot THF (50 mL×2). The combined filtrate was concentrated in vacuo to afford N-methyl-2-(4-methyl-1H-indol-3-yl)ethan-1-amine (1.59 g, 83%) as an off-white solid. ¹H NMR (400 MHz, DMSO-d₆): δ 10.75 (s, 1H), 7.14 (d, J=8.0 Hz, 1H), 7.05 (d, J=2.2 Hz, 1H), 6.97-6.84 (m, 1H), 6.68 (d, J=6.8 Hz, 1H), 2.96 (t, J=7.6 Hz, 2H), 2.73 (t, J=7.6 Hz, 2H), 2.61 (s, 3H), 2.33 (s, 3H). ¹³C NMR (101 MHz, DMSO-d₆): δ 136.7, 129.5, 125.6, 122.6, 120.7, 119.8, 113.4, 109.3, 53.9, 36.2, 27.1, 20.0.

### Step 3: N-methyl-N-(2-(4-methyl-1H-indol-3-yl)ethyl)propan-2-amine (P-162)

To a solution of N-methyl-2-(4-methyl-1H-indol-3-yl)ethan-1-amine (250 mg, 1.33 mmol) and acetone (84.8 mg, 1.1 eq., 1.46 mmol) in 1,2-dichloroethane (5 mL) was added NaBH(OAc)₃ (422 mg, 1.5 eq., 1.99 mmol) at ambient temperature. The reaction was stirred at ambient temperature for 16 h, then quenched with 1 M aq. NaOH (5 mL) and extracted with CH₂Cl₂ (10 mL×3). The combined organic layers were washed with brine (20 mL) and then concentrated under reduced pressure. The residue was purified by flash chromatography (0.1% to 6% MeOH/NH₃(aq.) in CH₂Cl₂) to afford N-methyl-N-(2-(4-methyl-1H-indol-3-yl)ethyl)propan-2-amine (190 mg) as a yellow oil which was used in the next step without further purification.

### Step 3a: N-methyl-N-(2-(4-methyl-1H-indol-3-yl)ethyl)propan-2-amine fumarate (P-162-fumarate)

To a solution of fumaric acid (96 mg, 0.83 mmol) in minimal refluxing acetone was added a solution of N-methyl-N-(2-(4-methyl-1H-indol-3-yl)ethyl)propan-2-amine (190 mg) in minimal warm acetone. The resulting solution was allowed to cool to ambient temperature and stood overnight at 4° C. to afford N-methyl-N-(2-(4-methyl-1H-indol-3-yl)ethyl)propan-2-amine as the fumarate salt (48 mg, 12% over 2 steps) which were colourless crystals. ¹H NMR (400 MHz, DMSO-d₆): δ 10.83 (s, 1H), 7.20-7.05 (m, 2H), 7.01-6.84 (m, 1H), 6.69 (d, J=7.2 Hz, 1H), 6.48 (s, 1H), 3.23-2.95 (m, 3H), 2.89-2.68 (m, 2H), 2.61 (s, 3H), 2.41 (s, 3H), 1.07 (d, J=6.8 Hz, 6H). ¹³C NMR (101 MHz, DMSO-d₆): δ 167.6, 136.6, 135.0, 129.3, 125.5, 122.9, 120.8, 119.9, 112.3, 109.4, 54.9, 53.9, 35.7, 24.2, 19.8, 17.0. qNMR Purity (ERETIC): 95.5%.

Functional Assays 5-HT_{2A}, 5-HT_{2B} and 5-HT_{2C} Receptors

Activity at 5-HT2A, 5-HT2B and 5-HT2C receptors was determined using a FLIPR Ca2+ flux assay at WuXi AppTec Co. Ltd. (Hong Kong) Discovery Biology Unit according to their standard protocols. Briefly, stably transfected cells expressing the receptor of interest (HEK293 for 5-HT2A and 5-HT2C; CHO-K1 for 5-HT2B) were grown and plated in a 384 well plate and incubated at 37° C. and 5% 002 overnight. A 250 mM stock solution of probenecid in FLIPR calcium assay buffer (10 mL) was freshly prepared and combined with a fluorescent dye (Fluo-4 Direct) to give a final assay concentration of 2.5 mM. Reference compounds were 4-fold serially diluted and the screening compounds were 3-fold serially diluted in 100% DMSO for 10 points using Agilent Bravo, and 750 nL was added to a 384 well compound plate using Echo along with 30 μL assay buffer. The fluorescent dye was then added to the assay plate along with assay buffer to a final volume of 40 μL. The cell plate was incubated for 50 ml at 37° C. and 5% 002 and placed into the FLIPR Tetra along with the compound plate. 10 μL of references and compounds were then transferred from the compound plate into the cell plate and the fluorescent signal was read.

TABLE 1
Agonist activity of exemplified compounds at selected s serotonin (5-HT)
receptors in Ca²⁺ flux functional assays.

| | | 5-HT2A | | 5-HT2B | | 5-HT2c | |
|---|---|---|---|---|---|---|---|
| | | EC₅₀ | Eₘₐₓ | EC₅₀ | Eₘₐₓ | EC₅₀ | Eₘₐₓ |
| Cmpd | Structure | (nM) | (%) | (nM) | (%) | (nM) | (%) |
| P-4 | 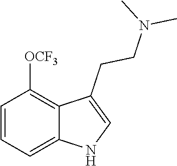 [SMILES: CN(C)CCc1c[nH]c2cccc(OC(F)(F)F)c12] | 52.83 | 34.57 | >10000 | −0.97 | 15.87 | 66.60 |
| P-5 | 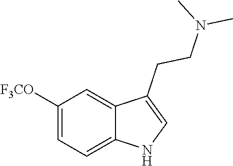 [SMILES: CN(C)CCc1c[nH]c2ccc(OC(F)(F)F)cc12] | 171.1 | 40.54 | >10000 | −0.27 | 520.4 | 55.10 |
| P-34 | 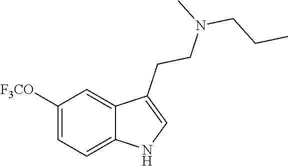 [SMILES: CCCN(C)CCc1c[nH]c2ccc(OC(F)(F)F)cc12] | 665.2 | 53.70 | >10000 | 18.89 | 329.1 | 81.70 |
| P-15 | 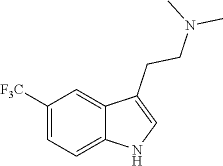 [SMILES: CN(C)CCc1c[nH]c2ccc(C(F)(F)F)cc12] | 185.7 | 28.69 | >10000 | 7.41 | 89.68 | 88.76 |
| P-42 | 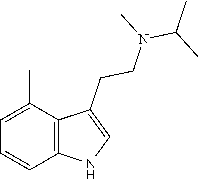 [SMILES: Cc1cccc2[nH]cc(CCN(C)C(C)C)c12] | 39.65 | 94.71 | 814.2 | 41.36 | 48.64 | 91.17 |
| P-16 | 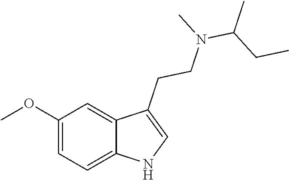 [SMILES: CCC(C)N(C)CCc1c[nH]c2ccc(OC)cc12] | 36.92 | 58.48 | >10000 | −1.20 | 553.2 | 59.52 |
| P-17 | 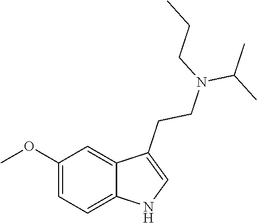 [SMILES: CCCN(CCc1c[nH]c2ccc(OC)cc12)C(C)C] | 13.82 | 89.04 | 137.9 | 69.19 | 184.9 | 82.14 |
| P-18 | 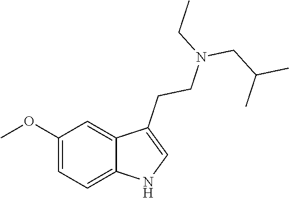 [SMILES: CCN(CCc1c[nH]c2ccc(OC)cc12)CC(C)C] | 47.93 | 79.45 | 867.2 | 51.79 | 1127 | 68.86 |
| P-19 | 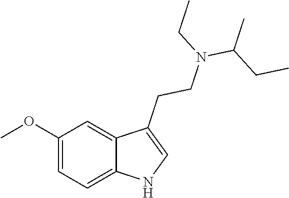 [SMILES: CCC(C)N(CC)CCc1c[nH]c2ccc(OC)cc12] | 33.31 | 70.27 | 1398 | 44.46 | 933.4 | 76.78 |
| P-20 | 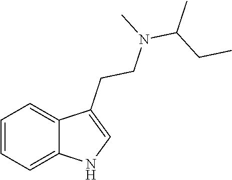 [SMILES: CCC(C)N(C)CCc1c[nH]c2ccccc12] | 91.17 | 16.10 | >10000 | 0.83 | 645.8 | 58.77 |
| P-21 | 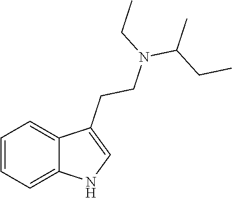 [SMILES: CCC(C)N(CC)CCc1c[nH]c2ccccc12] | 147.9 | 71.46 | >10000 | 37.20 | 973.5 | 61.49 |
| P-22 | 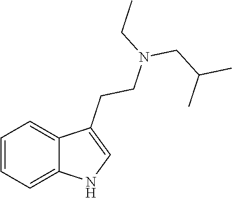 [SMILES: CCN(CCc1c[nH]c2ccccc12)CC(C)C] | 389.1 | 52.76 | 1135 | 28.04 | 959.7 | 58.43 |
| P-23 | 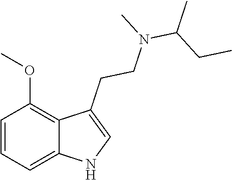 [SMILES: CCC(C)N(C)CCc1c[nH]c2cccc(OC)c12] | 123.8 | 50.27 | >10000 | −0.21 | 464.7 | 21.66 |
| P-24 | 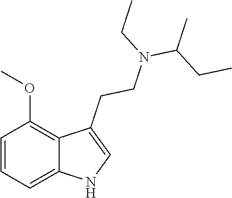 [SMILES: CCC(C)N(CC)CCc1c[nH]c2cccc(OC)c12] | 70.15 | 77.71 | 444.1 | 52.58 | 964.1 | 68.61 |
| P-25 | 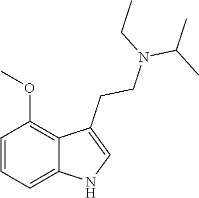 [SMILES: CCN(CCc1c[nH]c2cccc(OC)c12)C(C)C] | 121.4 | 85.89 | 459.4 | 61.40 | 676.1 | 77.81 |
| P-26 | 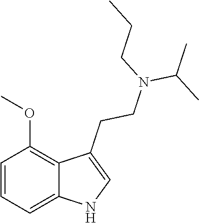 [SMILES: CCCN(CCc1c[nH]c2cccc(OC)c12)C(C)C] | 36.53 | 81.60 | 102.9 | 95.98 | 105.9 | 87.09 |
| P-136 | 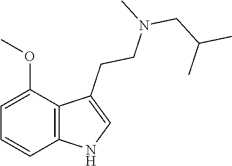 [SMILES: COc1cccc2[nH]cc(CCN(C)CC(C)C)c12] | 91.52 | 100.46 | >10000 | −1.63 | 261.10 | 78.53 |
| P-137 | 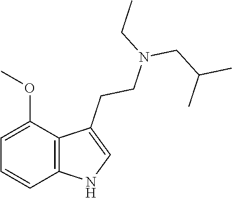 [SMILES: CCN(CCc1c[nH]c2cccc(OC)c12)CC(C)C] | 85.61 | 95.80 | >10000 | 19.40 | 137.9 | 96.25 |
| P-138 | 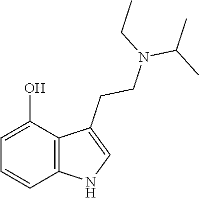 [SMILES: CCN(CCc1c[nH]c2cccc(O)c12)C(C)C] | 19.91 | 105.48 | 322.30 | 53.55 | 861.60 | 83.32 |
| P-139 | 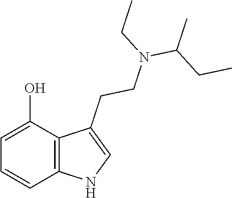 [SMILES: CCC(C)N(CC)CCc1c[nH]c2cccc(O)c12] | 53.98 | 107.47 | >10000 | 15.73 | 1902.00 | 55.16 |
| P-140 | 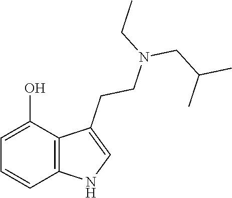 [SMILES: CCN(CCc1c[nH]c2cccc(O)c12)CC(C)C] | 27.70 | 97.47 | >10000 | 17.54 | 991.9 | 95.41 |
| P-141 | 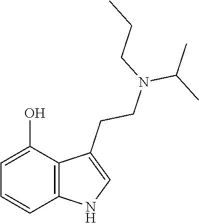 [SMILES: CCCN(CCc1c[nH]c2cccc(O)c12)C(C)C] | 13.80 | 104.84 | 330.00 | 62.17 | 421.50 | 92.01 |
| P-162 | 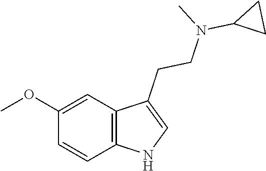 [SMILES: COc1ccc2[nH]cc(CCN(C)C3CC3)c2c1] | 4.827 | 95.61 | 10000 | 12.01 | 10.64 | 89.20 |
| P-163 | 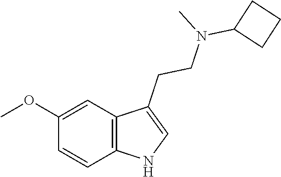 [SMILES: COc1ccc2[nH]cc(CCN(C)C3CCC3)c2c1] | 25.4 | 90.45 | 10000 | 2.29 | 127.5 | 75.92 |
| P-164 | 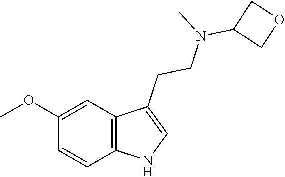 [SMILES: COc1ccc2[nH]cc(CCN(C)C3COC3)c2c1] | 671.2 | 57.50 | 10000 | 1.26 | 6281 | 59.94 |

### In Vivo Pharmacokinetics Experiments

Prospective studies for in vivo pharmacokinetics will be conducted using established procedures in accordance with the Australian Code of Practice for the Care and Use of Animals for Scientific Purposes, and the study protocols will be reviewed and approved by the Monash Institute of Pharmaceutical Sciences Animal Ethics Committee. A brief outline of a standard, previously used method is described below:

The systemic exposure of selected examples will be studied in non-fasted male C57BL/6 mice weighing between 18.9-25.5 g. Mice will have access to food and water ad libitum throughout the pre- and post-dose sampling period.

On the day of dosing, the formulation of each compound will be prepared by dissolving solid compound in an appropriate solvent using vortexing.

Compounds will be dosed to mice by IP injection (10 mL/kg dose volume via a 27 G needle; n=9 mice per compound) and blood samples will be collected at various time points (e.g. 5 and 30 min; 1, 2 and 4 h post-dose (n=3 mice per time point for each compound)). A maximum of three blood samples will be obtained from each mouse, with plasma samples being taken via submandibular bleed (approximately 120 μL). Once collected, blood samples will be processed by standard methods and analysed by LCMS. In addition, whole brain samples will be taken by rapid removal from the carcass soon after the blood collection. The whole brains will be blotted to remove excess blood, placed into pre-weighed polypropylene vials, and weighed. The brains will be snap frozen in dry ice and subsequently stored frozen (−80° C.) until analysis.

### Bioanalytical Method Summary:

Concentrations of test compound in plasma and tissue samples will be determined using an LCMS/MS method validated for linearity, accuracy, precision, matrix factor and recovery. Test compound standard solutions will be diluted from a concentrated stock solution (32 mM in H₂O) using 50% ACN in H₂O (v/v) and a calibration curve was prepared in a matched matrix to the test samples.

Plasma: The plasma calibration curve will be prepared by spiking aliquots of blank mouse plasma (25 μL) with test compound standard solutions (5 μL) and internal standard solution (5 μL of diazepam, 5 μg/mL in 50% acetonitrile in water). Test plasma samples (25 μL) will be thawed, mixed, and then spiked with internal standard solution (5 μL). Plasma protein precipitation can be performed by addition of acetonitrile (3-fold volume ratio) and thorough vortex mixing. Samples will be centrifuged (RCF=9391×g) for 3 minutes and the supernatant (90 μL) collected for analysis.

Tissue: Pre-weighed tissue samples (brain) will be prepared according to general methods, for example: homogenised using a glass rod in buffer containing an EDTA/potassium fluoride solution (0.1 M/4 mg/mL) as a stabilisation cocktail to minimise the potential for ex vivo degradation (3 mL cocktail/g tissue). The tissue homogenate will be briefly centrifuged (RCF=79×g) for 10 seconds to separate the foam layer before transferring an aliquot of the tissue homogenate (200 μL) to a fresh Eppendorf tube for sample extraction. Calibration standards will be prepared by spiking blank brain homogenate (200 μL) with the solution standards (10 μL) and the internal standard (10 μL). Study samples can be similarly prepared, except that acetonitrile (10 μL) was added instead of solution standards to maintain the same volume. Protein precipitation can be carried out by the addition of a 3-fold volume of acetonitrile, followed by vortex mixing and centrifugation (RCF=9391×g) for 3 min to recover the supernatant for analysis.

Replicate analysis: Triplicate analytical replicate (ARs) samples will be prepared similarly to the standards for each sample type at three concentrations (50, 500 and 2,000 ng/mL) and repeat injections of these ARs will be included throughout the analytical run to assess assay performance. The extraction of the test compound from the standards and ARs will be conducted as described above.

All test samples will be quantified within the calibration range of the assay and the stability of each test compound will be confirmed in homogenate during the period of sample processing (15 min; <15% loss).

### Biotelemetry and Head-Twitch Response (HTR) Experiments

Psychedelic potential will be assessed via the industry standard head twitch response in Mice (C₅₇BL/6J males). The study will be conducted through standard means of which are summarised below. Mice will be purchased from the Jackson Laboratory (Bar Harbor, ME, USA) at 5-6 weeks of age and allowed at least 1-2 weeks to acclimate to the animal research facility. Mice will be initially group housed 3-5 per cage during acclimation and housed in a 12 h light-dark cycle throughout the study, with lights on at 0700 h. Food and water will be available ad libitum except during testing. Cohorts of 20-24 mice will be used for each test drug. The mice will be subjected to experimental testing once every 1-2 weeks for 2-3 months to complete dose-effect curves and antagonist experiments. A minimum of 7 days between treatments will be utilized to avoid any tolerance to effects of repeated drug administration. All drug doses represent the weight of the salt dissolved in 0.9% saline vehicle. Mice will be tested first in dose-response studies to assess the effects of each compound at doses from 0.03 to 30 mg/kg s.c. and will be subsequently tested in antagonist reversal studies utilizing pretreatment with M100907 and WAY100635. All experiments will be conducted from 0900 to 1700 local time during the light phase, as sensitivity of rodents to other tryptamine psychedelics is diurnal, with maximal HTR observed in the middle of the light phase. Experiments will be run during the light phase also to avoid any potential influence of melatonin receptor activity on HTR as melatonin and related agonists are known to reduce HTR induced by DOI in rats. For each experiment, mice will be acclimated to the testing room in their home cage for at least 1 h prior to experimental sessions. Behavioral test sessions will be carried out in Tru Scan mouse locomotor arenas equipped with photobeam arrays (Coulbourn Instruments, Holliston, MA, USA), which will be modified with cylindrical inserts and transparent floors useful in detecting mouse HTR.

Subcutaneous Temperature Transponder Implants. At least 1 week prior to the start of the experiments, mice will recieve s.c. implanted temperature transponders (14×2 mm, model IPTT-300, Bio Medic Data Systems, Inc., Seaford, DE, USA) under brief isoflurane anesthesia. Mice were single housed post implant for the remainder of the study to protect the transponder from removal by cage mates. Temperature will be determined noninvasively using a handheld receiver that is sensitive to signals emitted from the implanted transponders.

Prior to each experiment, mouse body weight and temperature will be recorded. Mice will then be placed into testing chambers for acclimation. In dose-response studies, after a brief 5 min acclimation, mouse body temperature will be recorded for baseline measurement, mice will receive s.c. injection of test substance or vehicle, and animals will be returned to the testing arena for 30 min. During the session, locomotor activity will be monitored via photobeam tracking of movements in the horizontal plane to yield distance traveled in centimeter. HTR will be monitored by the analysis of GoPro Hero Black 7 video recordings (120 frames per sec and 960p resolution) using a commercially available software package from Clever Sys Inc. (Reston, VA, USA).82 post-treatment body temperature values will also be recorded, and temperature data is represented as change from pretreatment baseline.

In antagonist reversal experiments, mice will recieve a s.c. injection of either receptor antagonists or vehicle and were returned to the testing chamber for 30 min. During this period, locomotor activity will be monitored to examine the potential effects of antagonist treatment on general behavior or movement. At 30 min after antagonist administration, mice will be given test drug or vehicle and returned to the chambers for an additional 30 min of video recording used for analyses.

All statistical analyses will be conducted using GraphPad Prism 9 (La Jolla, CA, USA). Dose-response data from mouse experiments will be analyzed using nonlinear regression, and potency values will be determined from the rising phase of the curves for HTR measures. For mouse studies, one-way ANOVA with Dunnett's post hoc test will be used to compare all conditions to vehicle controls (0 or 0,0) in dose-response and antagonist experiments. Mean HTR count, distance traveled, and temperature change for each condition will be used for statistical comparisons. Alpha will be set at 0.05 for all analyses.

### Acute Restraint Stressor (ASR) Tail Suspension Test (TST) in Mice

Compounds will be assessed in a validated model of depression conducted the leading contract research organisation; Europhins Panlabs. The assessment will be conducted according to standard conditions and are briefly outlined Here: Male ICR mice (23±3 g) will be purchased from BioLASCO (Taipei, Taiwan) at 4-5 weeks of age and allowed 5-7 days to acclimate to the animal research facility at Pharmacology Discovery Services (Taipei, Taiwan). Mice will be housed in groups of 10 in a large cage (47×25×15 cm) on a 12-hour light cycle (lights on: 0700) and provided ad libitum food and water except during acute restraint stress and tail-suspension testing. Temperature will be maintained at 20-24° C., and all rooms (colony and testing rooms) had similar lighting intensity. All aspects of this work including housing, experimentation, and animal disposal were performed in accordance with the “Guide for the Care and Use of Laboratory Animals: Eighth Edition” (The National Academies Press, Washington, D C, 2011) in a facility accredited by the Association for Assessment and Accreditation of Laboratory Animal Care. All experiments will be conducted between 0900 to 1700 local time, during the light phase. Each mouse will undergo a single behavioural experiment in which they were randomly allocated to receive a single treatment with vehicle (50 mM phosphate buffered saline, pH=6.5), Ketamine as a positive control (10 mg/kg, diluted in 0.9% saline from 50 mg/ml stock), or one dose of a test drug (n=10 per dose of test drug, n=12 for vehicle, n=12 for ketamine). All drug doses represent the freebase dose in salt form dissolved in vehicle. All solutions will be delivered at 5 ml/kg via intraperitoneal injection.

Acute Restraint Stress (ARS) Procedure: Mice will be moved from the colony room to the procedure room in which ARS will be performed. Mice will recieve oral gavage of water (10 ml/kg) to avoid dehydration, and then will be individually restrained for 5 hours in a clear plastic cylinder (50 mL centrifuge tube with air holes drilled for ventilation), positioned horizontally on a bench with bench towel to absorb urine. This restraint will prevent physical movement, without causing pain. Restrainers will be washed with veterinary disinfectant between mice.

Drug Administration: Immediately after the 5-hour ARS procedure, mice will be removed from the restrainers, placed in their home cage, and transported to the room in which Tail Suspension Test will be be conducted. Mice will then receive intraperitoneal injection with vehicle, ketamine (10 mg/kg), test compounds (over a range of doses), and will be placed back in their home cage. 10 minutes after treatment, animals will undergo the Tail Suspension Test.

Tail Suspension Test (TST) Procedure: Mice will be individually suspended on the edge of a shelf, 58 cm above a tabletop, using adhesive tape placed approximately 1 cm from the tip of the tail, for a total duration of 7 minutes. Using a stopwatch, the experimenters blinded to treatment groups will record the duration of immobility (defined as hanging passively and motionless) during the 5 minutes spanning from 2-7 minutes. The data from 0-2 minutes will not be recorded. Mice undergoing TST will never be in view of other mice. Following TST, mice will be euthanized via carbon dioxide inhalation.

Statistical Analysis: Statistical analyses will be conducted using GraphPad Prism 9 (La Jolla, CA, USA), using a priori simple effect comparisons within a one-way ANOVA to compare the test compounds to the Vehicle condition, on time spent immobile (in seconds).

1. A compound of formula (I): 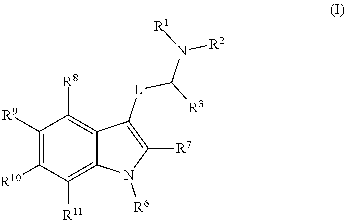
or a pharmaceutically acceptable salt, solvate, tautomer, N-oxide, stereoisomer, metabolite, polymorph or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O)R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²,
said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein one of (B) and (C) apply:
(B)
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl;
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃,
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form 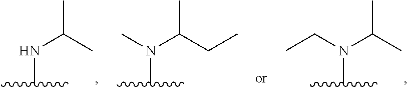
and R³ and R⁶ are each hydrogen, then R⁹ is not OCH₃;
when R¹ and R² together with the nitrogen to which they are attached form 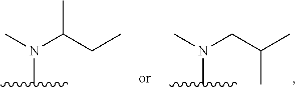
and R³ and R⁶ are each hydrogen, then R⁸ is not OH;
when R¹ and R² together with the nitrogen to which they are attached form 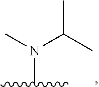 [SMILES: *N(C)C(C)C]
and R³ and R⁶ are each hydrogen, then R⁸ is not OH, R⁹ is not CH₃ or OCH₃, and R¹⁰ is not OCH₃;
when R¹ and R² together with the nitrogen to which they are attached form 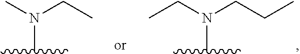
and R³ and R⁶ are each hydrogen, then R⁷, R⁸, R⁹, R¹⁰ and R¹¹ are not selected from OH, OCH₃, OC(O)CH₃, OP(O)(OH)₂, NH₂, halogen, CH₃, CN and CF₃;
when R¹ and R² together with the nitrogen to which they are attached form any one of 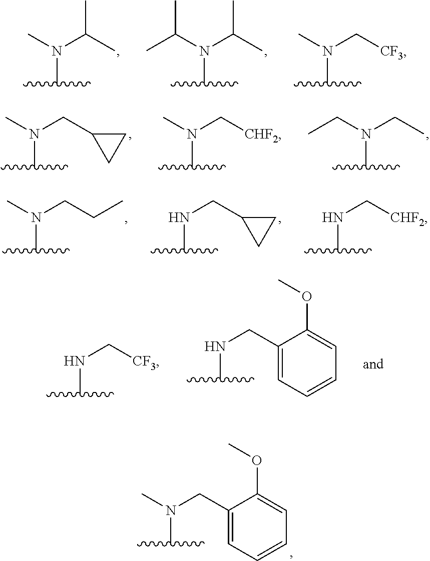
R³ is hydrogen, and R⁶ is methyl, then R⁸ is not OH or OBn;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH or OBn;
when R¹ and R² are each methyl, R³ is hydrogen, and R⁶ is hydrogen or CH₂P(O)(OH)₂, then R⁸ is not selected from 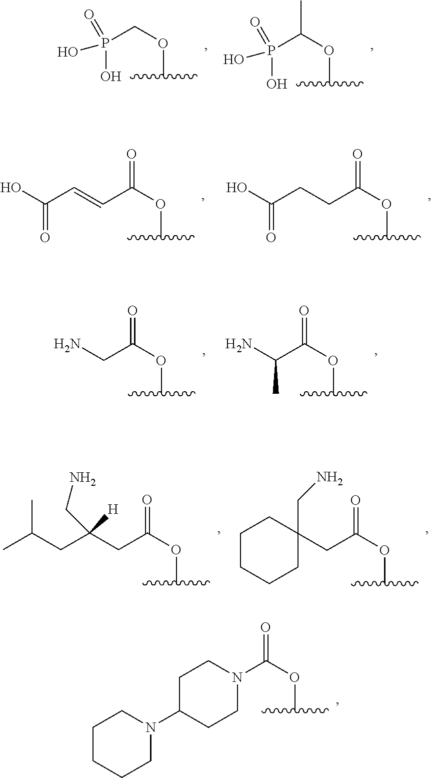
and OC(O)N(CH₃)₂; and
when R¹, R² and R³ together with the atoms to which they are attached form 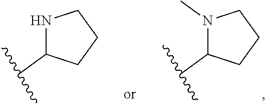
and R⁶ is hydrogen or CH₂P(O)(OH)₂, then R⁸ is not selected from 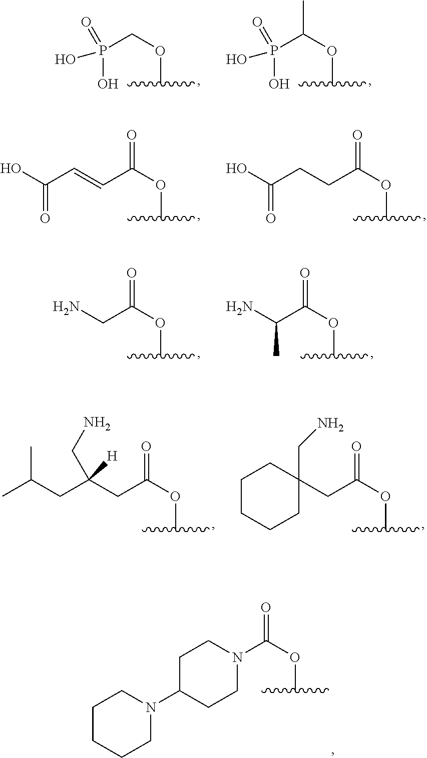
and O(O)N(CH₃)₂;
(C) R⁷, R⁸, R⁹, R¹⁰ and R¹¹ are each hydrogen,
wherein:
when R³ and R⁶ are each hydrogen, then R¹ and R² are not each methyl, ethyl,
propyl, isopropyl, cyclopropyl or 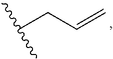 [SMILES: *CC=C]
and R¹ and R² together with the nitrogen to which they are attached do not form pyrrolidyl, piperidyl or 2,5-dimethylpyrrolyl;
when R⁶ is hydrogen, and R³ is methyl, then R¹ and R² are not each hydrogen;
when R³ and R⁶ are each hydrogen, and one of R¹ and R² is methyl, then the other of R¹ and R² is not propyl, isopropyl, cyclopropyl, methylenecyclopropyl, 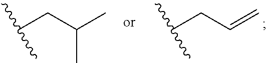
when R³ and R⁶ are each hydrogen, and one of R¹ and R² is ethyl or propyl, then the other of R¹ and R² is not isopropyl, cyclopropyl, methylenecyclopropyl, 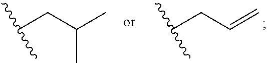
and
when R³ and R⁶ are each hydrogen, and one of R¹ and R² is isopropyl, then the other of R¹ and R² is not propyl, cyclopropyl, methylenecyclopropyl,

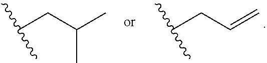

2. The compound of claim 1, wherein:
R⁷, R⁸, R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and one of R⁹, R¹⁰ or R¹¹ is fluoro and the other of R⁹, R¹⁰ or R¹¹ are hydrogen, then R⁸ is not selected from OH, OCH₃, OCH₂CH₃, OCH₂CH₂CH₃ and OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn.

3. The compound of claim 2, wherein:
R⁷, R⁸ R⁹, R¹⁰ and R¹¹ are each independently selected from hydrogen, halogen, CN, OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)N(R¹³)₂, OC(O)R¹³, OSO₂R¹³, OP(O)(OR¹³)₂, OC₁₋₆alkyleneP(O)(OR¹³)₂, S(O)R¹³, SO₂R¹³, N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NO₂, NHCH₃, SH, SCH₃, SO₂CH₃, and SOCH₃,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, NH and NCH₃;
wherein R¹³ is as defined in claim 2;
wherein at least two or more of R⁷, R⁸, R⁹, R¹⁰ and R¹¹ are not hydrogen; and
wherein:
when R¹ and R² are each methyl, R³ is hydrogen, R⁶ is selected from hydrogen, methyl, ethyl and propyl, and when one of R⁹, R¹⁰ and R¹¹ is fluoro and the other of R⁹, R¹⁰ and R¹¹ are hydrogen, then R⁸ is not selected from OH, OCH₃, OCH₂CH₃, OCH₂CH₂CH₃ or OBn; and
when R¹ and R² are each methyl, R³ is hydrogen, R⁶ is selected from hydrogen, methyl, ethyl and propyl, R⁹ is fluoro, and R¹¹ is hydrogen, then R¹⁰ is not selected from OH, OCH₃, OCH₂CH₃, OCH₂CH₂CH₃ and OBn.

4. The compound of claim 3, wherein R⁷, R⁸, R⁹, R¹⁰ and R¹¹ are each independently selected from hydrogen, halogen, C₁₋₆ alkyl, C₁₋₆ haloalkyl and OR¹³ wherein R¹³ is selected from hydrogen, C₁₋₆ alkyl and C₁₋₆ haloalkyl.

5. The compound of claim 4, wherein R⁸ is selected from halogen, C₁₋₆ alkyl and OR¹³ wherein R¹³ is selected from hydrogen, C₁₋₆ alkyl and C₁₋₆ haloalkyl.

6. The compound of claim 4, wherein R⁹ is selected from halogen, C₁₋₆ alkyl and OR¹³ wherein R¹³ is selected from hydrogen, C₁₋₆ alkyl and C₁₋₆ haloalkyl.

7. The compound of claim 1, wherein:
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆alkynyl, C₁-C₆ haloalkyl, C₃-C₇cycloalkyl, C₃₋₁₀ heterocycloalkyl, C₆₋₁₂ aryl and C₅₋₁₀ heteroaryl;
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein:
when R¹ and R² are each methyl, and R³ and R⁶ are each hydrogen, then R⁸ is not OH or OCH₃, and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form 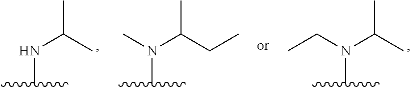
and R³ and R⁶ are each hydrogen, then R⁹ is not OCH₃;
when R¹ and R² together with the nitrogen to which they are attached form 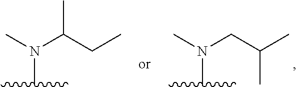
and R³ and R⁶ are each hydrogen, then R⁸ is not OH;
when R¹ and R² together with the nitrogen to which they are attached form 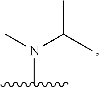 [SMILES: *N(C)C(C)C]
and R³ and R⁶ are each hydrogen, then R⁸ is not OH, R⁹ is not CH₃ or OCH₃, and R¹⁰ is not OCH₃;
when R¹ and R² together with the nitrogen to which they are attached form 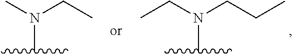
and R³ and R⁶ are each hydrogen, then R⁷, R⁸, R⁹, R¹⁰ and R¹¹ are not selected from OH, OCH₃, OC(O)CH₃, OP(O)(OH)₂, NH₂, halogen, CH₃, CN and CF₃;
when R¹ and R² together with the nitrogen to which they are attached form any 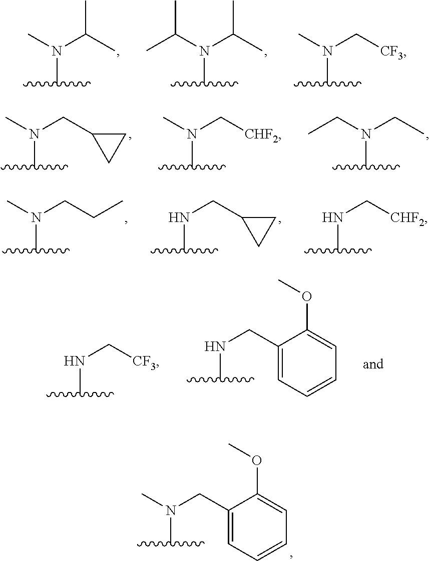
R³ is hydrogen, and R⁶ is methyl, then R⁸ is not OH or OBn;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH or OBn;
when R¹ and R² are each methyl, R³ is hydrogen, and R⁶ is hydrogen or CH₂P(O)(OH)₂, then R⁸ is not selected from 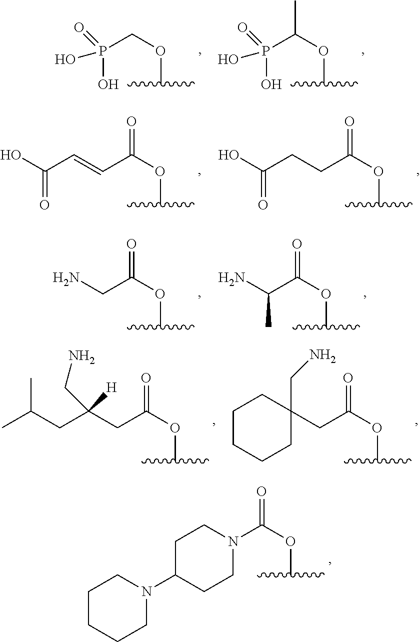
and OC(O)N(CH₃)₂; and
when R¹, R² and R³ together with the atoms to which they are attached form 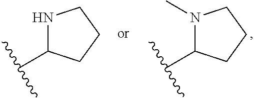
and R⁶ is hydrogen or CH₂P(O)(OH)₂, then R⁸ is not selected from 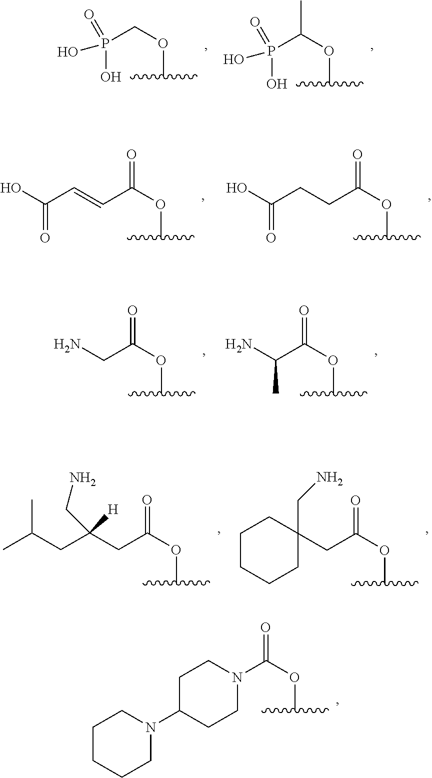
and OC(O)N(CH₃)₂.

8. The compound of claim 7, wherein:
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form 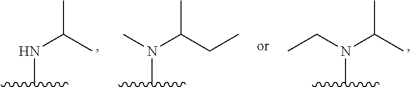
and R³ and R⁶ are each hydrogen, then R⁹ is not OCH₃;
when R¹ and R² together with the nitrogen to which they are attached form 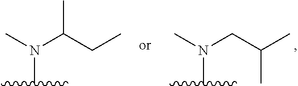
and R³ and R⁶ are each hydrogen, then R⁸ is not OH;
when R¹ and R² together with the nitrogen to which they are attached form 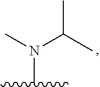 [SMILES: *N(C)C(C)C]
and R³ and R⁶ are each hydrogen, then R⁸ is not OH, R⁹ is not CH₃ or OCH₃, and R¹⁰ is not OCH₃;
when R¹ and R² together with the nitrogen to which they are attached form 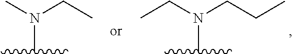
and R³ and R⁶ are each hydrogen, then R⁷, R⁸, R⁹, R¹⁰ and R¹¹ are not selected from OH, OCH₃, OC(O)CH₃, OP(O)(OH)₂, NH₂, halogen, CH₃, CN and CF₃;
when R¹ and R² together with the nitrogen to which they are attached form any one of 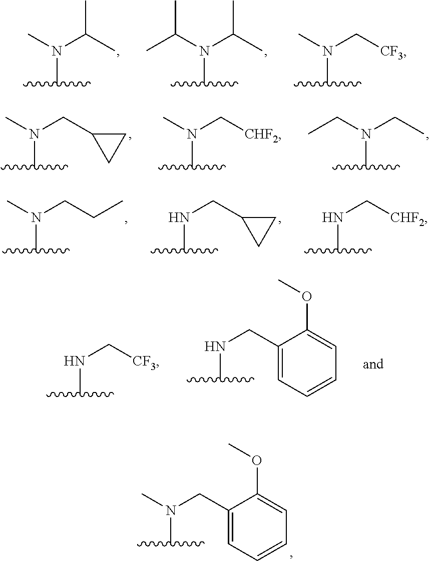
R³ is hydrogen, and R⁶ is methyl, then R⁸ is not OH;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH;
when R¹ and R² are each methyl, R³ is hydrogen, and R⁶ is hydrogen or CH₂P(O)(OH)₂, then R⁸ is not selected from 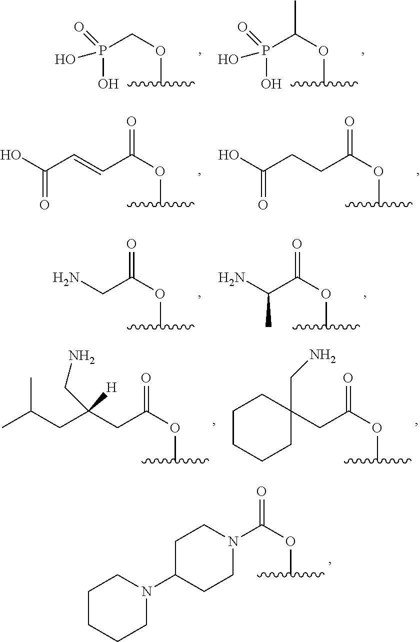
and OC(O)N(CH₃)₂; and
when R¹, R² and R³ together with the atoms to which they are attached form 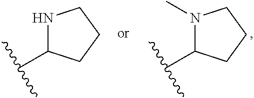
and R⁶ is hydrogen or CH₂P(O)(OH)₂, then R⁸ is not selected from 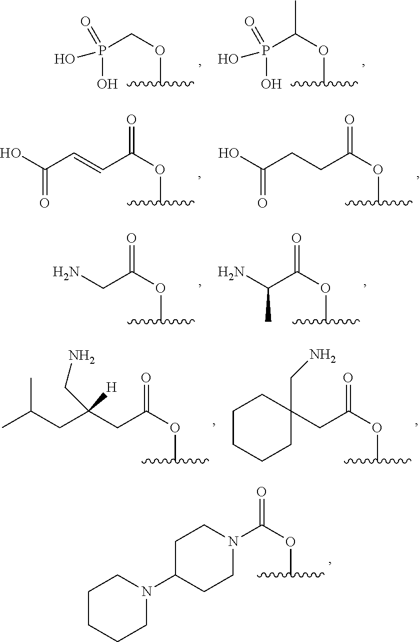
and OC(O)N(CH₃)₂.

9. The compound of claim 8, wherein:
(i) one of R⁷, R⁸, R⁹, R¹⁰ and R¹¹ is selected from C₁₋₆ haloalkyl and OC₁₋₆ haloalkyl, wherein the C₁₋₆ haloalkyl is not CF₃ when R¹ and R² together with the nitrogen to which they are attached form 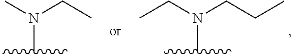
and R³ and R⁶ are each hydrogen; and
(ii) the other of R⁷, R⁸, R⁹, R¹⁰ and R¹¹ are each hydrogen.

10. The compound of claim 1, wherein:
R⁷, R⁸, R⁹, R¹⁰ and R¹¹ are each hydrogen,
wherein:
when R³ and R⁶ are each hydrogen, then R¹ and R² are not each methyl, ethyl, propyl, isopropyl, cyclopropyl or 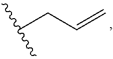 [SMILES: *CC=C]
and R¹ and R² together with the nitrogen to which they are attached do not form pyrrolidyl, piperidyl or 2,5-dimethylpyrrolyl;
when R⁶ is hydrogen, and R³ is methyl, then R¹ and R² are not each hydrogen;
when R³ and R⁶ are each hydrogen, and one of R¹ and R² is methyl, then the other of R¹ and R² is not propyl, isopropyl, cyclopropyl, methylenecyclopropyl, 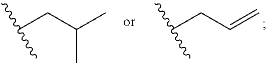
when R³ and R⁶ are each hydrogen, and one of R¹ and R² is ethyl or propyl, then the other of R¹ and R² is not isopropyl, cyclopropyl, methylenecyclopropyl 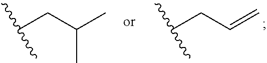
and
when R³ and R⁶ are each hydrogen, and one of R¹ and R² is isopropyl, then the other of R¹ and R² is not propyl, cyclopropyl, methylenecyclopropyl,

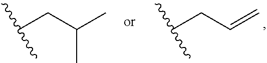

11. The compound of any one of claims 1 to 10, wherein R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl and C₄₋₁₄ alkylenecycloalkyl.

12. The compound of claim 11, wherein R¹ and R² are each independently selected from C₁₋₄ alkyl.

13. The compound of claim 12, wherein R¹ and R², together with the nitrogen to which they are attached, form any one of the following:

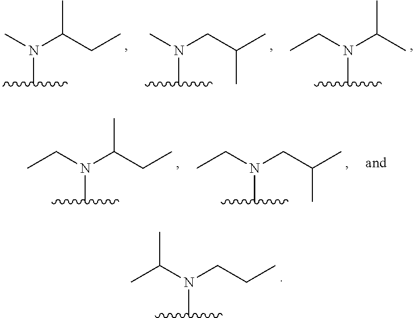

14. The compound of any one of claims 1 to 13, wherein R³ is hydrogen.

15. The compound of any one of claims 1 to 14, wherein L is C₁₋₄ alkylene.

16. The compound of claim 15, wherein L is methylene.

17. The compound of any one of claims 1 to 16, wherein R⁶ is selected from hydrogen and C₁₋₆ alkyl.

18. The compound of claim 17, wherein R⁶ is hydrogen.

19. The compound of claim 1 selected from any one of the compounds of Table 1 or a pharmaceutically acceptable salt, solvate, tautomer, N-oxide, stereoisomer, metabolite, polymorph or prodrug thereof.

20. A medicament comprising a compound of any one of claims 1 to 19, or a pharmaceutically acceptable salt, solvate, tautomer, N-oxide, stereoisomer, metabolite, polymorph or prodrug thereof.

21. A pharmaceutical composition comprising a compound of any one of claims 1 to 19, or a pharmaceutically acceptable salt, solvate, tautomer, N-oxide, stereoisomer, metabolite, polymorph or prodrug thereof, and a pharmaceutically acceptable excipient.

22. A method of treating a disease, disorder or condition by activation of a serotonin receptor, the method comprising administering to a subject in need thereof a compound of formula (I): 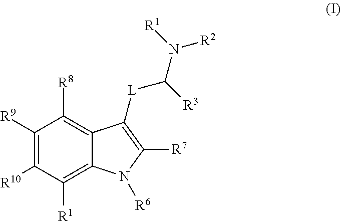
or a pharmaceutically acceptable salt, solvate, tautomer, N-oxide, stereoisomer, metabolite, polymorph or prodrug thereof,
wherein
R¹ and R² are each independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl, C₄₋₁₄ alkylenecycloalkyl, C₃-C₈ heterocycloalkyl, C₄-C₁₄ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴ and SO₂R⁴,
said C₃₋₈ cycloalkyl, C₄₋₁₄ alkylenecycloalkyl, C₃-C₈ heterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
alternatively R¹ and R² are combined with the atoms to which they are attached to form a C₃₋₈ heterocycloalkyl including 1 or 2 additional ring heteromoieties selected from O, S, S(O), SO₂, N and NR⁴,
said C₃₋₈ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆alkenyl, C₂₋₆ haloalkenyl, C₂₋₆ alkynyl, C₂₋₆ haloalkynyl, C₁₋₈ alkylamino, C₁₋₈ alkylsulfonyl, C₃₋₆ cycloalkyl and C₃₋₆ heterocycloalkyl including 1 or 2 ring heteromoieties selected from O, S, N, S(O), SO₂ and NR⁴;
R³ is selected from hydrogen, C₁₋₆ alkyl, C₃₋₈ cycloalkyl, or C₄₋₁₄ alkylenecycloalkyl;
alternatively R³ and one of R¹ and R² are combined with the atoms to which they are attached to form a C₃₋₁₂ heterocycloalkyl,
said C₃₋₁₂ heterocycloalkyl being further optionally substituted with a substituent selected from halogen, (O), CN, C₁₋₈ alkoxy, C₁₋₈ alkylamino, C₁₋₈ alkylsulfonyl, CO₂R⁴, C(O)N(R⁴)₂, OR⁴, N(R⁴)₂, NO₂, SR⁴, SO₂R⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR⁴;
each R⁴ is independently selected from hydrogen, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl, and C₃₋₇ heterocycloalkyl including 1 or 2 ring heteromoieties selected from O, S, S(O), SO₂, N and NR⁵,
said C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₇ cycloalkyl and C₃₋₇ heterocycloalkyl each being optionally substituted with one or more substituents independently selected from halogen, CN, C₁₋₈ alkoxy, C₁₋₈ alkylamino, C₁₋₈ alkylsulfonyl, CO₂R⁵, C(O)N(R⁵)₂, OR⁵, N(R⁵)₂, NO₂, SR⁵ and SO₂R⁵,
said C₃-C₇ cycloalkyl and C₃₋₇ heterocycloalkyl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N and NR⁵;
each R⁵ is independently selected from hydrogen, C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl,
said C₁₋₆ alkyl, C₂₋₆ alkenyl, C₂₋₆ alkynyl, C₁₋₆ haloalkyl, C₃₋₈ cycloalkyl, C₅₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
L is selected from C₁₋₄ alkylene, C₂-C₄ alkenylene and C₂-C₄ alkynylene;
R⁶ is selected from hydrogen, C₁₋₆ alkyl, C₂₋₆ alkenyl, C₂₋₆ alkynyl, C₁₋₆ alkyleneP(O)(OR¹²)₂, C(O)R¹², CO₂R¹², C(O)N(R¹²)₂, S(O)R¹² and SO₂R¹², C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂R¹², C(O)N(R¹²)₂, OR¹², N(R¹²)₂, NO₂, SR¹² and SO₂R¹²,
said C₃₋₆ cycloalkyl, C₆₋₉ alkylenecycloalkyl, C₃₋₆ heterocyclyl, C₆₋₉ alkyleneheterocycloalkyl, C₄₋₇ heterocyclyl, C₇₋₁₀ alkyneneheterocycloalkyl, C₆₋₁₂ aryl, C₇₋₁₈ alkylenearyl, C₅₋₁₀ heteroaryl, and C₆₋₁₆ alkyleneheteroaryl each being further optionally substituted with a substituent independently selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹²;
each R¹² is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein one of (B) and (C) apply:
(B)
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆alkynyl, C₁-C₆ haloalkyl, C₃-C₇ cycloalkyl, C₃₋₁₀ heterocycloalkyl, C₆₋₁₂ aryl and C₅₋₁₀ heteroaryl,
said C₁₋₆ alkyl, C₂-C₆ alkenyl, C₂-C₆ alkynyl, C₁-C₆ haloalkyl, C₃-C₇ cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form 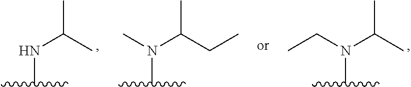
and R³ and R⁶ are each hydrogen, then R⁹ is not OCH₃;
when R¹ and R² together with the nitrogen to which they are attached form 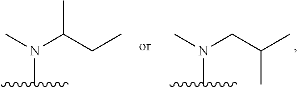
and R³ and R⁶ are each hydrogen, then R⁸ is not OH;
when R¹ and R² together with the nitrogen to which they are attached form 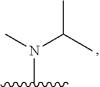 [SMILES: *N(C)C(C)C]
and R³ and R⁶ are each hydrogen, then R⁸ is not OH, R⁹ is not CH₃ or OCH₃, and R¹⁰ is not OCH₃;
when R¹ and R² together with the nitrogen to which they are attached form any one of 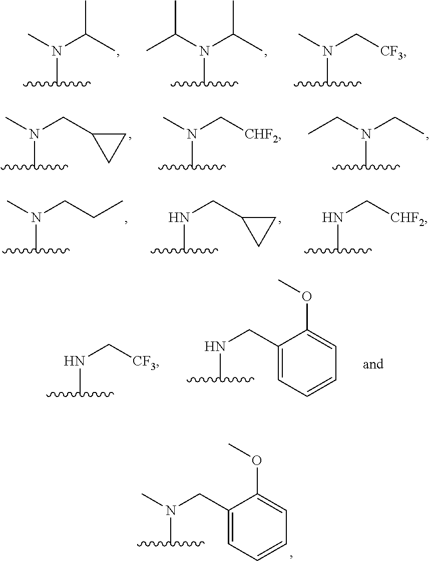
is hydrogen, and R⁶ is methyl, then R⁸ is not OH;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH;
when R¹ and R² are each methyl, R³ is hydrogen, and R⁶ is hydrogen or CH₂P(O)(OH)₂, then R⁸ is not selected from 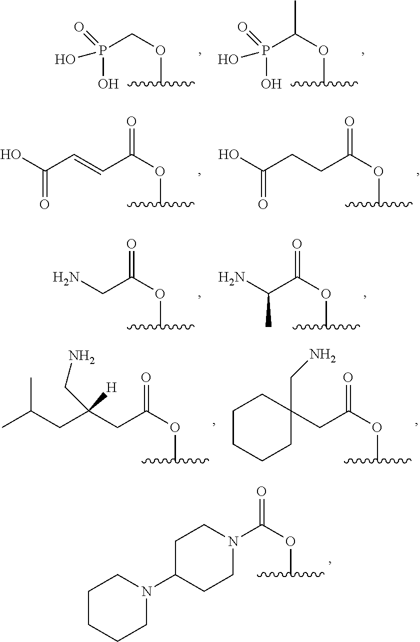
and OC(O)N(CH₃)₂; and
when R¹, R² and R³ together with the atoms to which they are attached form 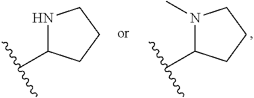
and R⁶ is hydrogen or CH₂P(O)(OH)₂, then R⁸ is not selected from 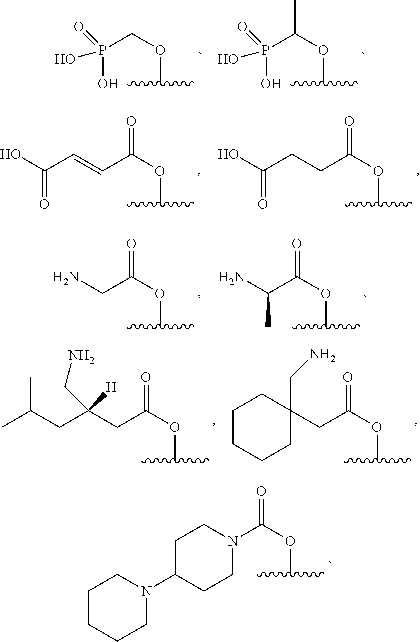
and OC(O)N(CH₃)₂;
(C) R⁷, R⁸, R⁹, R¹⁰ and R¹¹ are each hydrogen,
wherein:
when R⁶ and R³ are each hydrogen, then R¹ and R² are not each methyl, and R¹ and R² together with the nitrogen to which they are attached do not form 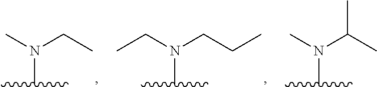
pyrrolidyl, piperidyl or 2,5-dimethylpyrrolyl; and
when R⁶ is hydrogen, and R³ is methyl, then R¹ and R² are not each hydrogen.

23. The method of claim 22, wherein:
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen,
alternatively, R⁶ and R⁷ are combined with the atoms to which they are each attached to form a C₄₋₁₀ heterocycloalkyl or a C₅₋₁₀ heteroaryl,
said C₄₋₁₀ heterocycloalkyl and C₅₋₁₀ heteroaryl each being further optionally substituted with a substituent selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
alternatively, R⁷ and one of R¹, R², or R³ are combined with the atoms to which they are attached to form a C₅₋₈ heterocycloalkyl,
said C₅₋₈ heterocyclyalkyl being further optionally substituted with one or more substituents selected from halogen, (O), CN, C₁₋₈ alkoxy, C₁₋₈ alkylamino, C₁₋₈ alkylsulfonyl, CO₂R¹⁴, C(O)N(R¹⁴)₂, OR¹⁴, N(R¹⁴)₂, NO₂, SR¹⁴, SO₂R¹⁴, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, N, S(O), SO₂ and NR¹⁴;
each R¹⁴ is independently selected from hydrogen, C₁₋₆ alkyl, C₂-C₆ alkenyl, C₂-C₆alkynyl, C₁-C₆ haloalkyl, C₃-C₇cycloalkyl, C₃₋₁₀ heterocycloalkyl, C₆₋₁₂ aryl and C₅₋₁₀ heteroaryl;
said C₁₋₆ alkyl, C₂-C₆ alkenyl, C₂-C₆ alkynyl, C₁-C₆ haloalkyl, C₃-C₇cycloalkyl, C₃₋₁₀ heterocycloalkyl, C₆₋₁₂ aryl and C₅₋₁₀ heteroaryl each being optionally substituted with one or more substituents independently selected from halogen, CN, C₁₋₈ alkoxy, C₁₋₈ alkylamino, C₁₋₈ alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form 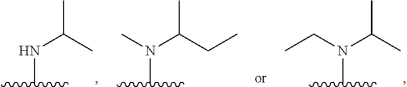
and R³ and R⁶ are each hydrogen, then R⁹ is not OCH₃;
when R¹ and R² together with the nitrogen to which they are attached form 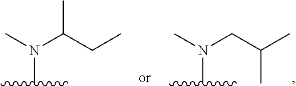
and R³ and R⁶ are each hydrogen, then R⁸ is not OH;
when R¹ and R² together with the nitrogen to which they are attached form 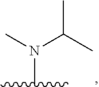 [SMILES: *N(C)C(C)C]
and R³ and R⁶ are each hydrogen, then R⁸ is not OH, R⁹ is not CH₃ or OCH₃, and R¹⁰ is not OCH₃;
when R¹ and R² together with the nitrogen to which they are attached form any one of 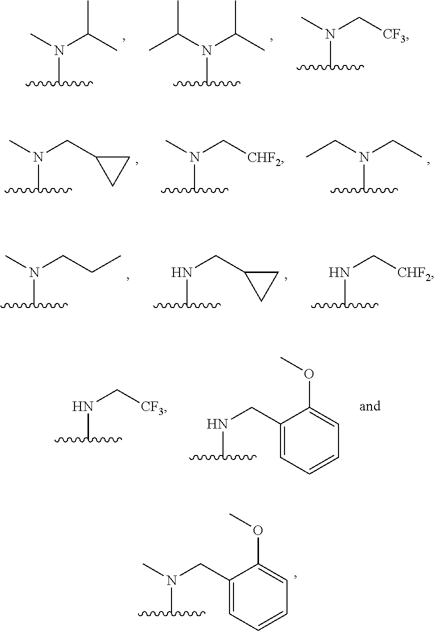
is hydrogen, and R⁶ is methyl, then R⁸ is not OH;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH;
when R¹ and R² are each methyl, R³ is hydrogen, and R⁶ is hydrogen or CH₂P(O)(OH)₂, then R⁸ is not selected from 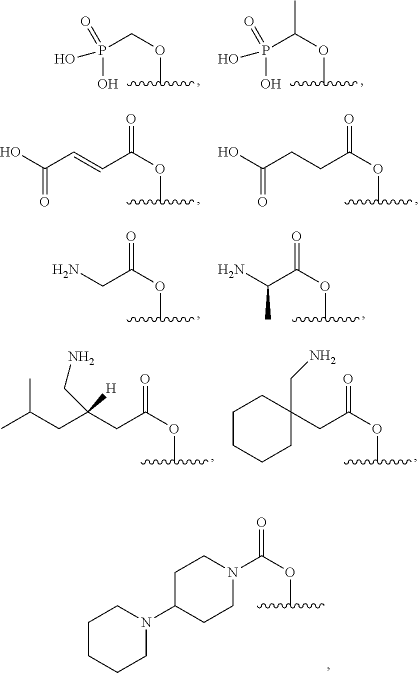
and OC(O)N(CH₃)₂; and
when R¹, R² and R³ together with the atoms to which they are attached form 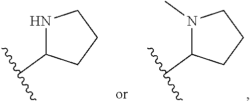
and R⁶ is hydrogen or CH₂P(O)(OH)₂, then R⁸ is not selected from 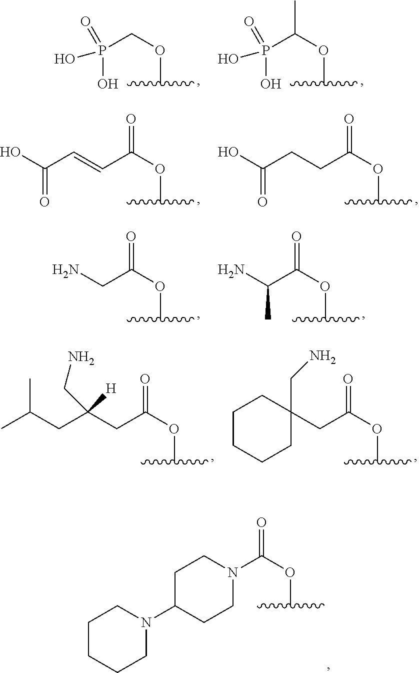
and OC(O)N(CH₃)₂.

24. The method of claim 23, wherein:
(i) one of R⁷, R⁸, R⁹, R¹⁰ and R¹¹ is selected from OR¹³, N(R¹³)₂, SR¹³, C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, CO₂R¹³, C(O)R¹³, C(O)N(R¹³)₂, C(O)C(O)N(R¹³)₂, OC(O)R¹³, OC(O)OR¹³, OC(O)N(R¹³)₂, OS(O)R¹³, OS(O)N(R¹³)₂, OSO₂R¹³, OP(O)(OR¹³)₂, OC₁₋₆alkyleneP(O)(OR¹³)₂, S(O)R¹³, S(O)N(R¹³)₂, SO₂R¹³, N(R¹³)₂, N(R¹³)C(O)R¹³, N(R¹³)C(O)OR¹³, N(R¹³)C(O)N(R¹³)₂, NO₂, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, C₄₋₁₆ alkyleneheteroaryl,
said C₁₋₆ alkyl, C₁₋₆ haloalkyl, C₂₋₆ alkenyl, C₂-C₆ haloalkenyl, C₂₋₆ alkynyl, C₂₋₆ haloalkynyl, C₁₋₆ alkylamine, C₁₋₆ alkoxy, C₁₋₆ haloalkoxy, C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl being optionally substituted with one or more substituents independently selected from halogen, CN, C₁₋₈ alkoxy, C₁₋₈ alkylamino, C₁₋₈ alkylsulfonyl, CO₂R¹³, C(O)N(R¹³)₂, OR¹³, N(R¹³)₂, NO₂, SR¹³ and SO₂R¹³,
said C₃₋₈ cycloalkyl, C₃₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₄₋₁₆ alkyleneheteroaryl each being further optionally substituted with a substituent selected from (O), C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆cycloalkyl and C₃₋₆ heterocycloalkyl including 1 or 2 ring heteromoeities selected from O, S, S(O), SO₂, N, and NR¹³;
each R¹³ is independently selected from hydrogen, C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl,
said C₁₋₆ alkyl, C₂₋₆ alkenyl, C₂₋₆ alkynyl, C₁₋₆ haloalkyl, C₃₋₈ cycloalkyl, C₄₋₁₄ alkylenecycloalkyl, C₃₋₁₀ heterocycloalkyl, C₄₋₁₆ alkyleneheterocycloalkyl, C₆₋₁₂ aryl, C₇₋₁₈ alkylenearyl, C₅₋₁₀ heteroaryl, and C₆₋₁₆ alkyleneheteroaryl each being optionally substituted with one or more substituents independently selected from halogen, CN, C₁₋₈ alkoxy, C₁₋₈ alkylamino, C₁₋₈alkylsulfonyl, CO₂H, CO₂CH₃, C(O)NH₂, C(O)N(CH₃)₂, C(O)NHCH₃, OH, NH₂, N(CH₃)₂, NHCH₃, NO₂, SH, SCH₃, SO₂CH₃, SOCH₃, C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₆ cycloalkyl and C₃₋₆ heterocycloalkyl including 1 or 2 ring heteromoieties selected from O, S, S(O), SO₂, N, NH and NCH₃; and
(ii) the other of R⁷, R⁸, R⁹, R¹⁰ and R¹¹ are each hydrogen;
wherein:
when R¹ and R² are each methyl, and R³ and R⁶ are each hydrogen, then R⁸ is not OH or OCH₃ and R⁹ is not OH;
when R¹ and R² are each ethyl, isobutyl or (sec)butyl, and R³ and R⁶ are each hydrogen, then R⁸ is not OH;
when R¹ and R² are each isopropyl, and R³ and R⁶ are each hydrogen, then R⁹ is not OH;
when R¹ and R² together with the nitrogen to which they are attached form 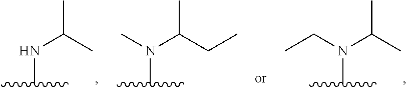
and R³ and R⁶ are each hydrogen, then R⁹ is not OCH₃;
when R¹ and R² together with the nitrogen to which they are attached form 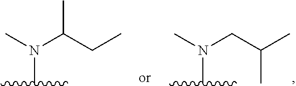
and R³ and R⁶ are each hydrogen, then R⁸ is not OH;
when R¹ and R² together with the nitrogen to which they are attached form 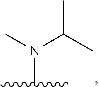 [SMILES: *N(C)C(C)C]
and R³ and R⁶ are each hydrogen, then R⁸ is not OH, R⁹ is not CH₃ or OCH₃, and R¹⁰ is not OCH₃;
when R¹ and R² together with the nitrogen to which they are attached form any one of 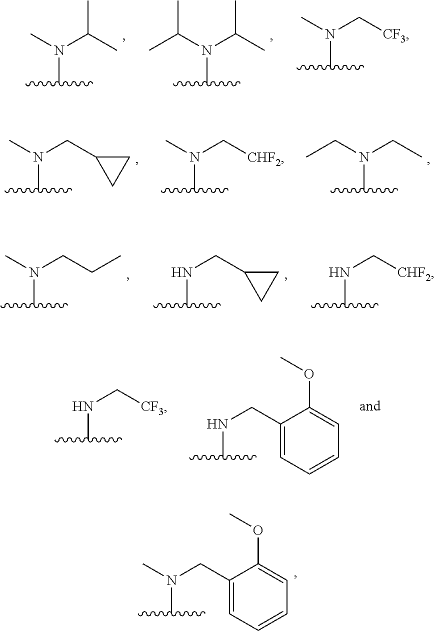
R³ is hydrogen, and R⁶ is methyl, then R⁸ is not OH;
when R¹ and R² are each methyl, R³ is hydrogen, and R⁶ is selected from ethyl, CH₂CHF₂, propyl, isopropyl, butyl, cyclopropyl, methylenecyclopropyl, cyclobutyl, oxetanyl and butenyl, then R⁸ is not OH;
when R¹ and R² are each methyl, R³ is hydrogen, and R⁶ is hydrogen or CH₂P(O)(OH)₂, then R⁸ is not selected from 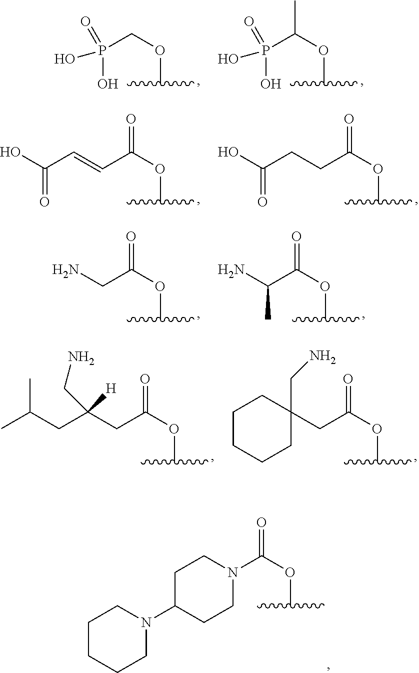
and OC(O)N(CH₃)₂; and
when R¹, R² and R³ together with the atoms to which they are attached form 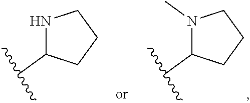
and R⁶ is hydrogen or CH₂P(O)(OH)₂, then R⁸ is not 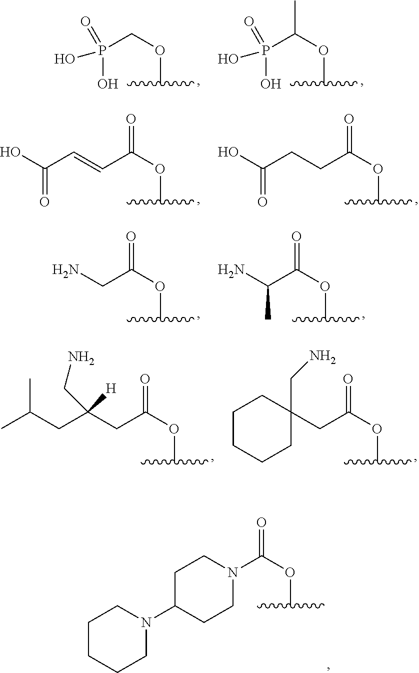
and OC(O)N(CH₃)₂.

25. The method of claim 24, wherein:
(i) one of R⁷, R⁸, R⁹, R¹⁰ and R¹¹ is selected from C₁₋₆ haloalkyl and OC₁₋₆ haloalkyl, and
(ii) the other of R⁷, R⁸, R⁹, R¹⁰ and R¹¹ are each hydrogen.

26. The method of claim 22, wherein:
R⁷, R⁸, R⁹, R¹⁰ and R¹¹ are each hydrogen,
wherein:
when R⁶ and R³ are each hydrogen, then R¹ and R² are not each methyl, and R¹ and R² together with the nitrogen to which they are attached do not form 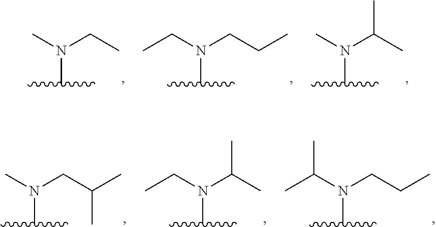
pyrrolidyl, piperidyl or 2,5-dimethylpyrrolyl; and
when R⁶ is hydrogen, and R³ is methyl, then R¹ and R² are not each hydrogen.

27. The method of any one of claims 22 to 26, wherein R¹ and R² are each independently selected from C₁₋₆ alkyl, C₁₋₆ haloalkyl, C₂₋₆ alkenyl, C₂₋₆ haloalkenyl, C₂₋₆ alkynyl, C₂₋₆ haloalkynyl, C₃₋₈ cycloalkyl and C₄₋₁₄ alkylenecycloalkyl.

28. The method of claim 27, wherein R¹ and R² are each independently selected from C₁₋₄ alkyl.

29. The method of claim 28, wherein R¹ and R², together with the nitrogen to which they are attached, form any one of the following:

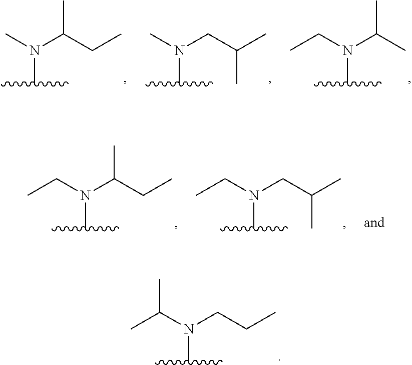

30. The method of any one of claims 22 to 29, wherein R³ is hydrogen.

31. The method of any one of claims 22 to 30, wherein L is C₁₋₄ alkylene.

32. The method of claim 31, wherein L is methylene.

33. The method of any one of claims 22 to 32, wherein R⁶ is selected from hydrogen and C₁₋₆ alkyl.

34. The method of claim 33, wherein R⁶ is hydrogen.

35. The method of claim 22, wherein the compound of formula (I) is selected from any one of compounds 4-5, 15-61 and 119-164 or a pharmaceutically acceptable salt, solvate, tautomer, N-oxide, stereoisomer, metabolite, polymorph or prodrug thereof.

36. A method of treating a disease, disorder or condition by activation of a serotonin receptor, the method comprising administering to a subject in need thereof a compound of formula (I) as defined in any one of claims 22 to 36, or a pharmaceutically acceptable salt, solvate, tautomer, N-oxide, stereoisomer, metabolite, polymorph or prodrug thereof, in combination with another known agent useful for treatment of a disease, disorder or condition by activation of a serotonin receptor.

37. A method of treating a mental illness, the method comprising administering to a subject in need thereof a compound of formula (I) as defined in any one of claims 22 to 36, or a pharmaceutically acceptable salt, solvate, tautomer, N-oxide, stereoisomer, metabolite, polymorph or prodrug thereof.

38. The method of claim 37, wherein the mental illness is selected from anxiety disorders; depression; mood disorders; psychotic disorders; impulse control and addiction disorders; drug addiction; obsessive-compulsive disorder (OCD); post-traumatic stress disorder (PTSD); stress response syndromes; dissociative disorders; depersonalization disorder; factitious disorders; sexual and gender disorders; somatic symptom disorders; hallucinations; delusions; psychosis; and combinations thereof.

39. A method for treating a central nervous system (CNS) disease, disorder or condition and/or a neurological disease, disorder or condition, the method comprising administering to a subject in need thereof a compound of formula (I) as defined in any one of claims 22 to 36, or a pharmaceutically acceptable salt, solvate, tautomer, N-oxide, stereoisomer, metabolite, polymorph or prodrug thereof.

40. The method of claim 39, wherein the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa and bulimia nervosa; binge eating disorder, trichotillomania, dermotillomania, nail biting; migraine; fibromyalgia; and peripheral neuropathy of any etiology, and combinations thereof.

41. A method for increasing neuronal plasticity and/or increasing dendritic spine density, the method comprising contacting a neuronal cell with a compound of formula (I) as defined in any one of claims 22 to 36, or a pharmaceutically acceptable salt, solvate, tautomer, N-oxide, stereoisomer, metabolite, polymorph or prodrug thereof, in an amount sufficient to increase neuronal plasticity and/or increase dendritic spine density of the neuronal cell.
